SEQUENCE LISTING (41 sequences)

SEQ ID NO: 1 - protein, 6 aa, Artificial Sequence
  Description of Artificial Sequence Synthetic Peptide
FWXMXW

SEQ ID NO: 2 - protein, 7 aa, Artificial Sequence
  Description of Artificial Sequence Synthetic Peptide
HCSAGXG

SEQ ID NO: 3 - protein, 6 aa, Artificial Sequence
  Description of Artificial Sequence Synthetic Peptide
FLERLE

SEQ ID NO: 4 - protein, 6 aa, Artificial Sequence
  Description of Artificial Sequence Synthetic Peptide
RWXMXW

SEQ ID NO: 5 - protein, 482 aa, Mus musculus
MRHSKRTYCPDWDERDWDYGTWRSSSSHKRKKRSHSSAREQKRCRYDHSKTTDSYYLESRSINEKAYHSRRYVDEYRNDYMGYEPGHPYGEPGSRYQMHSSKSSGRSGRSSYKSKHRSRHHTSDHHSHGHSHRRKRSRSVEDDEEGHLICQSGDVLSARYEIVDTLGEGAFGKVVECIDHKVGGRRVAVKIVKNVDRYCEAAQSEIQVLEHLNTTDPHSTFRCVQMLEWFEHRGHICIVFELLGLSTYDFIKENSFLPFRMDHIRKMAYQICKSVNFLHSNKLTHTDLKPENILFVKSDYTEANPKMKRDERTIVNPDIKVVDFGSATYDDEHHSTLVSTRHYRAPEVILALGWSQPCDVWSIGCILIEYYLGFTVFPTHDSREHLAMMERILGPLPKHMIQKTRKRRYFHHDRLDWDEHSSAGRYVSRRCKPLKEFMLSQDAEHEFLFDLVGKILEYDPAKRITLKEALKHPFFYPLKKHT

SEQ ID NO: 6 - DNA, 27 bp, Artificial Sequence
  Description of Artificial Sequence Synthetic Primer
ctctgtgtccacagcagtgctggctgt

SEQ ID NO: 7 - protein, 7 aa, Artificial Sequence
  Description of Artificial Sequence Synthetic Peptide
HRDLAAR

SEQ ID NO: 8 - protein, 6 aa, Artificial Sequence
  Description of Artificial Sequence Synthetic Peptide
DXWSXG

SEQ ID NO: 9 - DNA, 28 bp, Artificial Sequence
  Description of Artificial Sequence Synthetic Primer
cgggatcccttcgccttgcagctttgtc

SEQ ID NO: 10 - DNA, 30 bp, Artificial Sequence
  Description of Artificial Sequence Synthetic Primer
cggaattcctagactgatacagtctgtaag

SEQ ID NO: 11 - protein, 6 aa, Artificial Sequence
  Description of Artificial Sequence Synthetic Peptide
DLKPEN

SEQ ID NO: 12 - protein, 6 aa, Artificial Sequence
  Description of Artificial Sequence Synthetic Peptide
AMMERI

SEQ ID NO: 13 - DNA, 30 bp, Artificial Sequence
  Description of Artificial Sequence Synthetic Primer
tatagcggccgctagactgatacagtctgt

SEQ ID NO: 14 - DNA, 32 bp, Artificial Sequence
  Description of Artificial Sequence Synthetic Primer
tcccccgggatgccccatccccgaaggtacca

SEQ ID NO: 15 - DNA, 39 bp, Artificial Sequence
  Description of Artificial Sequence Synthetic Primer
tatagcggccgctcaccgactgatatcccgactggagtc

SEQ ID NO: 16 - DNA, 30 bp, Artificial Sequence
  Description of Artificial Sequence Synthetic Primer
tcccccggggagacgatgcatcactgtaag

SEQ ID NO: 17 - DNA, 39 bp, Artificial Sequence
  Description of Artificial Sequence Synthetic Primer
tatagcggccgcgctggcctgcacctgtcatctgctggg

SEQ ID NO: 18 - DNA, 30 bp, Artificial Sequence
  Description of Artificial Sequence Synthetic Primer
cggaattcatgcggcattccaaacgaactc

SEQ ID NO: 19 - DNA, 39 bp, Artificial Sequence
  Description of Artificial Sequence Synthetic Primer
tatagcggccgccctgactcccactcatttcctttttaa

SEQ ID NO: 20 - DNA, 36 bp, Artificial Sequence
  Description of Artificial Sequence Synthetic Primer
cggaattccgccaccatggcccctatactaggttat

SEQ ID NO: 21 - DNA, 36 bp, Artificial Sequence
  Description of Artificial Sequence Synthetic Primer
gccaagcttgccaccatggcccctatactaggttat

SEQ ID NO: 22 - DNA, 21 bp, Artificial Sequence
  Description of Artificial Sequence Synthetic Primer
gtagcagtaagaatagttaaa

SEQ ID NO: 23 - DNA, 24 bp, Artificial Sequence
  Description of Artificial Sequence Synthetic Primer
gttgccctgaggatcattaagaat

SEQ ID NO: 24 - DNA, 24 bp, Artificial Sequence
  Description of Artificial Sequence Synthetic Primer
gttgccctgaggatcatccggaat

SEQ ID NO: 25 - DNA, 30 bp, Artificial Sequence
  Description of Artificial Sequence Synthetic Primer
tacaattctcactgctacatgtaagccatc

SEQ ID NO: 26 - protein, 13 aa, Artificial Sequence
  Description of Artificial Sequence Synthetic Peptide
PIYSFIGGEHFPR

SEQ ID NO: 27 - protein, 9 aa, Artificial Sequence
  Description of Artificial Sequence Synthetic Peptide
IVEPDTEIK

SEQ ID NO: 28 - protein, 6 aa, Artificial Sequence
  Description of Artificial Sequence Synthetic Peptide
YGFSPR

SEQ ID NO: 29 - protein, 12 aa, Artificial Sequence
  Description of Artificial Sequence Synthetic Peptide
IKEVAHVNLEVR

SEQ ID NO: 30 - protein, 8 aa, Artificial Sequence
  Description of Artificial Sequence Synthetic Peptide
VAAGDSAT

SEQ ID NO: 31 - DNA, 2226 bp, Rattus rattus
  CDS (28)..(1386)
gaattccggcacgaggcgggttgcagtatgagtcgccaatcggacctagtgaggagcttcttggagcagcaggaggcccgggaccaccggaagggggcaatcctcgcccgtgagttcagcgacattaaggcccgctcagtggcttggaagactgaaggtgtgtgctccactaaagccggcagtcagcagggaaactcaaagaagaaccgctacaaagacgtggtaccgtatgatgagacgagagtcatcctttccctgctccaggaggaaggacacggagattacattaatgccaacttcatccggggcacagatggaagccaggcctacattgcgacgcaaggacccctgcctcacactctgttggacttctggcgcctggtttgggagtttggaatcaaggtgatcttgatggcctgtcaggagacagaaaatggacggaggaagtgtgaacgctactgggcccaggagcgggagcctctacaggccgggcctttctgcatcaccctgacaaaggagacagcactgacttcggacatcactctcaggaccctccaggttacattccagaaggaatcccgtcctgtgcaccagctacagtacatgtcttggccggaccacggggttcccagcagttccgatcacattctcaccatggtggaggaggcccgttgcctccaaggacttggacctggacccctctgtgtccactgcagtgctggctgtggacgaacaggtgtcttgtgtgctgttgattacgtgaggcagttgcttctgactcagacaatcccacccaatttcagcctctttgaagtggtcctggagatgcggaaacagcgacctgcagcggtgcagacagaggagcagtacaggttcctgtaccacacagtggctcagctattctcccgcactctccagaacaacagtcccctctaccagaacctcaaggagaaccgcgctccaatctgcaaggactcctcgtccctcaggacctcctcagccctgcctgccacatcccgcccactgggtggcgttctcaggagcatctcggtgcctgggccaccgacccttcccatggctgacacttacgctgtggtgcagaagcgtggcgcttccggcagcacagggccgggcacgcgggcgcccaacagcacggacaccccgatctacagccaggtggctccacgtatccagcggcccgtgtcacacaccgaaaacgcgcaggggacaacggcactgggccgagttcctgcggatgaaaacccttccgggcctgatgcctatgaggaagtaacagatggagcgcagactggtgggctaggcttcaacttgcgcattggaagacctaaagggccacgggatcctccagcggagtggacacgggtgtaatgagtgctgtaccagttccagcctgtcactcagtggtggctgggcgactgcaacccccatgctgctgtgtgctgtcttatgtatgagtgggactcatgggcctgaatcaaaataaaagtttctcagggtagaaaaaaacaaatagggactttggccagtggttatagcagtcaaagccaggggctaggaggggtaagtgggggaggtggtggatctactctgagaaagtttaggaaagcacatcaagagtgagcatcgccactcttctccccatacacctactggaaagtgcaccccagacagagtcctaacttgacagtgcacctcagacaggtcgctacctggatggacatgctggccctacagctagagacatgtctaattagatcctcatgtaaacttgcaatgagctagaaagatctccgtctggtcagggaaatggatcacctagtcaggtaaatagtgtgccatccagaagacagaactgcaagataccgtctttctcaaaatggaagaaaatagatcctcaagaataaatgtatgtacaatgctctacgccctgatcctgccctgcctcactgccataatgtcacaaacaagtcagggtctatatgacagttgttcatctagtcagtcctgactgtggcctctgcaggctcagatagtgccttctgcagactcttggaatgcccgtcttgaacttgatgaaagcttctaccgggaacttgtaaacatcattaaaattattaatgtagaattcaataaagagtgggtcaaaaactcaaaaaaaaaaaaaaaaaaaaaaaaaaactcgagagtacttctagagcgggcggg

SEQ ID NO: 32 - protein, 453 aa, Rattus rattus
MSRQSDLVRSFLEQQEARDHRKGAILAREFSDIKARSVAWKTEGVCSTKAGSQQGNSKKNRYKDVVPYDETRVILSLLQEEGHGDYINANFIRGTDGSQAYIATQGPLPHTLLDFWRLVWEFGIKVILMACQETENGRRKCERYWAQEREPLQAGPFCITLTKETALTSDITLRTLQVTFQKESRPVHQLQYMSWPDHGVPSSSDHILTMVEEARCLQGLGPGPLCVHCSAGCGRTGVLCAVDYVRQLLLTQTIPPNFSLFEVVLEMRKQRPAAVQTEEQYRFLYHTVAQLFSRTLQNNSPLYQNLKENRAPICKDSSSLRTSSALPATSRPLGGVLRSISVPGPPTLPMADTYAVVQKRGASGSTGPGTRAPNSTDTPIYSQVAPRIQRPVSHTENAQGTTALGRVPADENPSGPDAYEEVTDGAQTGGLGFNLRIGRPKGPRDPPAEWTRV

SEQ ID NO: 33 - DNA, 5581 bp, Unknown Organism
  Description of Unknown Organism Mammalian PCP-2 sequence
aattccgggcgccagtcccgctccgcgccgcgccgctccgctccggctcgggctccggctcgcctcgggctgggctcgggctccgggggcggcgtccccgcgccgggccccgggacgcgccgacctccaaccatggcccgtgcccaggcgctcgtgctggcactcaccttccagctctgcgcgccggagaccgagactccggcagctggctgcaccttcgaggaggcaagtgacccagcagtgccctgcgagtacagccaggcccagtacgatgacttccagtgggagcaagtgcgaatccaccctggcacccgggcacctgcggacctgccccacggctcctacttgatggtcaacacttcccagcatgccccaggccagcgagcccatgtcatcttccagagcctgagcgagaatgatacccactgtgtgcagttcagctacttcctgtacagccgggacggcacaggcggcaccctgcgcgtctacgtgcgcgttaatgggggccccctggcgagtgctgtgtggaatatgactggatcccacggccgtcagtggcaccaggctgagctggctgtcagcactttctggcccaatgaatatcaggtgctgtttgaggccctcatctccccagaccgcaggggctacatgggcctagatgacatcctgcttctcagctacccctgcgcaaaggccccacacttctcccgcctgggcgacgtggaggtcaacgcgggccagaacgcgtcgttccagtgcatggccgcgggagagcccatgcgccaacgcttcctcttgcaacggcagagcggggccctggtgccggccggggcgttcggcacatcagccaccggcttcctggccactttcccgctggctgccgtgagccgcgccgagcaggacctgtaccgctgtgtgtcccaggccccgcgcggcggcgtctctaacttcccggagctcatcgtcaaggagcccccaactcccatcgcgcccccacagctgctgcgtgctggccccacctacctcatcatccagctcaacaccaactccatcattggcgacgggccgatcgtgcgcaaggagattgagtaccgcatggcgcgcgggccctgggctgaggtgcacgccgtcagcctgcagacctacaagctgtggcacctcgaccccgacacagactatgagatcagcgtgctgctcacgcgtcccggagacggcggcactggccgctgggccacccctcatcagccgcaccaaatgcgcagagcccatgagggccccaaaggcctggcttttgctgagatccaggcccgtcagctgaccctgcagtgggaaccactgggctacaacgtgacgcgttgccacacctatactgtgtcgctgtgctatcactacaccctgggcagcagccacaaccagaccatccgagagtgtgtgaagacagagcaaggtgtcagccgctacaccatcaagaacctgctgccctatcggaacgttcacgtgaggcttgtcctcactaaccctgaggggcgcaaagagggcaaggaggtcactttccagacggatgaggatgtgcccagtgggattgcagccgagtccctgaccttcactccactggaggacatgatcttcctcaagtgggaggagccccaggagcccaatggtctcatcacccagtatgagatcagctaccagagcatcgagtcatcagacccggcagtgaacgtgccaggcccacgacgtaccatctccaagctccgcaatgagacctaccatgtcttctccaacctgcacccaggcaccacctacctgttctccgtgcgggcccgcacaggcaaaggcttcggccaggcggcactcactgagataaccactaacatctctgctcccagctttgattatgccgacatgccgtcacccctgggcgagtctgagaacaccatcaccgtgctgctgaggccggcacagggccgcggtgcgcccatcagtgtgtaccaggtgattgtggaggaggagcgggcgcgaggctgcggcgggacgaggtggacaggactgcttcccagtgccattgaccttcgaggcggcgctggccccaggctggtgcactacttcggggccgaactggcggccagcagtctacctgaggccatgccctttaccgtgggtgacaaccagacctaccgaggcttctggaacccaccacttgagcctaggaaggcctatctcatctacttccaggcagcaagccacctgaagggggagacccggctgaattgcatccgcattgccaggaaagctgcctgcaaggaaagcaagcggcccctggaggtgtcccagagatcggaggagatggggcttatcctgggcatctgtgcaggggggcttgctgtcctcatccttctcctgggtgccatcattgtcatcatccgcaaagggaagccggtgaacatgaccaaggccaccgtcaactaccgccaggagaagacacacatgatcagcgccgtggaccgcagcttcacagaccagagcaccctgcaggaggacgagcggctgggcctgtccttcatggacacccatggctacagcacccggggagaccagcgcagcggtggggtcactgaggccagcagcctcctggggggctccccgaggcgtccctgtggccggaagggctccccataccacacggggcagctgcaccctgcggtgcgtgtcgcagaccttctgcagcacatcaaccagatgaagacggccgagggttacggcttcaagcaggagtatgagagcttctttgaaggctgggacgccacaaagaagaaagacaaggtcaagggcagccggcaggagccaatgcctgcctatgatcggcaccgagtgaaactgcacccgatgctgggagaccccaatgccgactacattaatgccaactacatagatggttaccacaggtcaaaccacttcatagccactcaagggccgaagcctgagatggtctatgacttctggcgtatggtgtggcaggagcactgttccagcatcgtcatgatcaccaagctggtcgaggtgggcagggtgaaatgctcacggtactggccggaggactcagacacctacggggacatcaagattatgctggtgaagacagagaccctggctgagtatgtcgtgcgcacttttgccctggagcggagaggctactctgcccggcacgaggtccgccagtcccacttcacagcgtggccagagcatggcgtcccctaccatgccacggggctgctggctttcatccggcgggtgaaggcctccaccccacctgatgccgggcccattgtcatccactgcagcgcgggcaccggccgcacacgttgctatatcgtcctggatgtgatgctggacatggcagagtgtgagggcgtcgtggacatttacaactgtgtgaagactctctgctcccggcgtgtcaacatgatccagactgaggagcagtacatcttcattcatgatgcaatcctggaggcctgcctgtgtggggagaccaccatccctgtcagtgagttcaaggccacctacaaggagatgatccgcattgatcctcagagtaattcctcccagctgcgggaagagttccagacgctgaactcggtcaccccgccgctggacgtggaggagtgcagcatcgccctgttgccccggaaccgcgacaagaaccgcagcatggacgtcctgccgcccgaccgctgcctgcccttcctcatctccactgatggggactccaacaactacattaatgcagccctgactgacagctacacacggaggtcggccttcatggtgaccctgcacccgctgcagagcaccacgcccgacttctggcggctggtctacgattacgggtgcacctccatcgtcatgctcaaccagctgaaccagtccaactccgcctggccctgcctgcagtactggccagagccaggccggcagcaatatggcctcatggaggtggagtttatgtcgggcacagctgatgaagacttagtggctcgagtcttccgggtgcagaacatctctcggttgcaggagggagacctgctggtgcggcacttccagttcctgcgctggtctgcataccgggacacacctgactccaagaaggccttcttgcacctgctggctgaggtggacaagtggcaggccgagagtggggatgggcgcaccatcgtgcactgcctaaacgggggaggacgcagcggcaccttctgcgcctgcgccacggtcctggagatgatccgctgccacaacttggtggacgttttctttgctgcccaaaccctccggaactacaaacccaacatggtggagaccatggatcagtaccacttttgctacgatgtggccctggagtacttggaggggctggagtcaagatagcggggccctggcctggggcacccactgcacactcagggccagacccaccatcctggactggcgaggaagatcagtgcctcctgctctgcccaaacacactcccatggggcaagcactggagtggatgctgggctatcttgctcccccttccactgtgggcagggcctttcgcttgtcccatgggcgggtggtgggccaaggaggagcttagcaagtctgcagcccacccccacctccatagggtcctgcaggcctgtgctgagaggcctggtgctgcctggcagagtgacaaaggctcaggacggctggctctgggggactcaggccaagggggttggcaggatcctgggttttggcagggatgagtgaggccctgcagagagcatcccaggccaaggttcccactcagcctgccccctctgcatgtgggtagaggatgtactgggacttggcatttaggattccatctgggggaccccctgaaggtcccccccaagcaggtctcaattctgatagccagtggggcacactcactgtcctccccaggggaactgcagcgccctcctccccactgccccctgcagcccctgagatattttgctcactatccctccccacttgcttccctgatatgtgctctgacttccctgaaccaggatctgcctattactgctgtcccatggggggctccttccctgcctgacccactgttgcagaatgaagtcacctcgcccccctcttcctttaatcttcaggcctcactggcctgtcctgctcagcttgggccagtgacaatctgcaaggctgaacaacagcccctggggttgaggcccctgtggctcctggtcaggctgcccgttgtggggaggggcagtgttagagcagggctggtcataccctctggagttcagagcaagaggtaggaccagtgcttttttgtttcttttgttatttttggttgggtgggtgggaaggtctctttaaaatggggcaggccacacccccattccgtgcctcaatttccccatctgtaaactgtagatatgactactgacctacctcgcagggggctgtggggaggcataagctgatgtttgtaaagcgctttgtaaataaacgtgctctctgaatgccaaaaaaaaaaaaacaaaaaaaa

SEQ ID NO: 34 - protein, 1430 aa, Unknown Organism
  Description of Unknown Organism Mammalian PCP-2 sequence
MARAQALVLALTFQLCAPETETPAAGCTFEEASDPAVPCEYSQAQYDDFQWEQVRIHPGTRAPADLPHGSYLMVNTSQHAPGQRAHVIFQSLSENDTHCVQFSYFLYSRDGTGGTLRVYVRVNGGPLASAVWNMTGSHGRQWHQAELAVSTFWPNEYQVLFEALISPDRRGYMGLDDILLLSYPCAKAPHFSRLGDVEVNAGQNASFQCMAAGEPMRQRFLLQRQSGALVPAGAFGTSATGFLATFPLAAVSRAEQDLYRCVSQAPRGGVSNFPELIVKEPPTPIAPPQLLRAGPTYLIIQLNTNSIIGDGPIVRKEIEYRMARGPWAEVHAVSLQTYKLWHLDPDTDYEISVLLTRPGDGGTGRWATPHQPHQMRRAHEGPKGLAFAEIQARQLTLQWEPLGYNVTRCHTYTVSLCYHYTLGSSHNQTIRECVKTEQGVSRYTIKNLLPYRNVHVRLVLTNPEGRKEGKEVTFQTDEDVPSGIAAESLTFTPLEDMIFLKWEEPQEPNGLITQYEISYQSIESSDPAVNVPGPRRTISKLRNETYHVFSNLHPGTTYLFSVRARTGKGFGQAALTEITTNISAPSFDYADMPSPLGESENTITVLLRPAQGRGAPISVYQVIVEEERARGCGGTRWTGLLPSAIDLRGGAGPRLVHYFGAELAASSLPEAMPFTVGDNQTYRGFWNPPLEPRKAYLIYFQAASHLKGETRLNCIRIARKAACKESKRPLEVSQRSEEMGLILGICAGGLAVLILLLGAIIVIIRKGKPVNMTKATVNYRQEKTHMISAVDRSFTDQSTLQEDERLGLSFMDTHGYSTRGDQRSGGVTEASSLLGGSPRRPCGRKGSPYHTGQLHPAVRVADLLQHINQMKTAEGYGFKQEYESFFEGWDATKKKDKVKGSRQEPMPAYDRHRVKLHPMLGDPNADYINANYIDGYHRSNHFIATQGPKPEMVYDFWRMVWQEHCSSIVMITKLVEVGRVKCSRYWPEDSDTYGDIKIMLVKTETLAEYVVRTFALERRGYSARHEVRQSHFTAWPEHGVPYHATGLLAFIRRVKASTPPDAGPIVIHCSAGTGRTRCYIVLDVMLDMAECEGVVDIYNCVKTLCSRRVNMIQTEEQYIFIHDAILEACLCGETTIPVSEFKATYKEMIRIDPQSNSSQLREEFQTLNSVTPPLDVEECSIALLPRNRDKNRSMDVLPPDRCLPFLISTDGDSNNYINAALTDSYTRRSAFMVTLHPLQSTTPDFWRLVYDYGCTSIVMLNQLNQSNSAWPCLQYWPEPGRQQYGLMEVEFMSGTADEDLVARVFRVQNISRLQEGDLLVRHFQFLRWSAYRDTPDSKKAFLHLLAEVDKWQAESGDGRTIVHCLNGGGRSGTFCACATVLEMIRCHNLVDVFFAAQTLRNYKPNMVETMDQYHFCYDVALEYLEGLESR

SEQ ID NO: 35 - DNA, 2810 bp, Homo sapiens
  CDS (44)..(1417)
gaattcggcacgagcgggctggaccttgctcgcccgcggcgccatgagccgcagcctggactcggcgcggagcttcctggagcggctggaagcgcggggcggccgggagggggcagtcctcgccggcgagttcagcgacatccaggcctgctcggccgcctggaaggctgacggcgtgtgctccaccgtggccggcagtcggccagagaacgtgaggaagaaccgctacaaagacgtgctgccttatgatcagacgcgagtaatcctctccctgctccaggaagagggacacagcgactacattaatggcaacttcatccggggcgtggatggaagcctggcctacattgccacgcaaggacccttgcctcacaccctgctagacttctggagactggtctgggagtttggggtcaaggtgatcctgatggcctgtcgagagatagagaatgggcggaaaaggtgtgagcggtactgggcccaggagcaggagccactgcagactgggcttttctgcatcactctgataaaggagaagtggctgaatgaggacatcatgctcaggaccctcaaggtcacattccagaaggagtcccgttctgtgtaccagctacagtatatgtcctggccagaccgtggggtccccagcagtcctgaccacatgctcgccatggtggaggaagcccgtcgcctccagggatctggccctgaacccctctgtgtccactgcagtgcgggttgtgggcgaacaggcgtcctgtgcaccgtggattatgtgaggcagctgctcctgacccagatgatcccacctgacttcagtctctttgatgtggtccttaagatgaggaagcagcggcctgcggccgtgcagacagaggagcagtacaggttcctgtaccacacggtggctcagatgttctgctccacactccagaatgccagcccccactaccagaacatcaaagagaattgtgccccactctacgacgatgccctcttcctccggactccccaggcacttctcgccataccccgcccaccaggaggggtcctcaggagcatctctgtgcccgggtccccgggccacgccatggctgacacctacgcggaggagcagaagcgcggggctccagcgggcgccgggagtgggacgcagacggggacggggacgggggcgcgcagcgcggaggaggcgccgctctacagcaaggtgacgccgcgcgcccagcgacccggggcgcacgcggaggacgcgagggggacgctgcctggccgcgttcctgctgaccaaagtcctgccggatctggcgcctacgaggacgtggcgggtggagctcagaccggtgggctaggtttcaacctgcgcattgggaggccgaagggtccccgggacccgcctgctgagtggacccgggtgtaagtctaacgccagttcctgcctgttgcctcttgtgagctcggactgctgatgccccggtgctgctgagcgccgtgccgagaatggaaacagtgggcctggatcaaagttaaagtttctcagggtgggaaatgtgggggctttgcccaatgactgtagcattcaaggcttgaggctggaggaggtagctagggtatagtggctggtgaggctgcacagagcagattcaagaaagaagatcaggaaggggcatgacccctgagttatgaaggggagaagggacagatgagcttccggagactgctctcctcaccacacagcactagtccatcctcagcacctgagcctccctcacttggacactcaggggaccacacagagaagtggatggacacttcgccatccaggcagaactaagccaggcataaccacagccaagcagattaaccccaggcagaccgataaaaagacctccagataggcagacagacagatggaccaccaacctggacagacagccaaagcttcagagatacagtccacaggtggacaaagggatccccagccagagagagagagaccagccaacagcttgatagaccagtgcagccagagagaccaccaaacacagcccccaaaagacagacatctctgctagctggacagccaggtggaccccctaagttagtcagattactagacagatataaacagatcccctgctgaacagatatacagagttctcagaccccactccctcaggtgggctggctggctgacagaccttctggccagacagactcctaaccaaccagatggactgccagacaggcagacatcagtccacatggaatcctgacatcccagccagccggccagactctcatcttgatgtcttgatggatggaccccagctagtcagacatgatcctccagattgacagacaagtcccccaaatgagtacacatctccagctattcagacagatggagccccagcaaatcaggacctatctaggcagaccccagccagacccccgccagacagactcccaaccagactgaccccttgctgttcacacagcctgccgagtagctgggactacaggtctaattttttttttttttaagaaatgagtttttgccatgttgcccagactggtcttgaactcccaacctcaagcaatcctcctgcctcagcctcccaaagtgctgagattacaggtgtgagccaccaggctcagccccctaagatttgaaacactttaaatggcccatggtagggttcctgctaggataaaacattaagtggctgttaaaagaaataaaaggaggacacgtctctgtgcaaaaaaaaaaaaaaaaaaaaaaaaaaaaaaaaaaaaaaaaaaaa

SEQ ID NO: 36 - protein, 458 aa, Homo sapiens [sequence omitted]

SEQ ID NO: 37 - protein, 503 aa, Unknown Organism
  Description of Unknown Organism Mammalian SIRP4 amino acid sequence
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 38 - protein, 398 aa, Unknown Organism
  Description of Unknown Organism Mammalian SIRP1 amino acid sequence
MPVPASWPHLPSPFLLMTLLLGRLTGVAGEDELQVIQPEKSVSVAAGESATLRCAMTSLIPVGPIMWFRGAGAGRELIYNQKEGHFPRVTTVSELTKRNNLNFSISISNITPADAGTYYCVKFRKGSPDDVEFKSGAGTELSVRAKPSAPVVSGPAVRATPEHTVSFTCESHGFSPRDITLKWFKNGNELSDFQTNVDPAGDSVSYSIHSTARVVLTRGDVHSQVICEMAHITLQGDPLRGTANLSEAIRVPPTLEVTQQPMRAENQANVTCQVSNFYPRGLQLTWLENGNVSRTETASTLIENKDGTYNWMSWLLVNTCAHRDDVVLTCQVEHDGQQAVSKSYALEISAHQKEHGSDITHEPALAPTAPLLVALLLGPKLLLVVGVSAIYICWKQKA

SEQ ID NO: 39 - protein, 495 aa, Mus musculus
PRPRKYHSSERGSRGSYHEHYQSRKHKRRRSRSWSSSSDRTRRRAREDSYHVRSRSSYDDHSSDRRLYDRRYCGSYRRNDYSRDRGEAYYDTDFRQSYEYHRENSSYRSQRSSRRKHRRRRRRSRTFSRSSSHSSRRAKSVEDDAEGHLIYHVGDWLQERYEIVSTLGEGTSGRVVQCIDRRVGTRRVLVIIKNVEKYKEAARLEINVLEKINEKDPKNKNLCVQMFDWFDYHGHMCISFELLGLSTFDFLKDNNYLPYPIHQVRHMAFQLCQAVKFLHDNKLTHTDLKPENILFVNSDYELTNPLEKRDERTSVKSTAVRVDFGSATYFDHHHSTLISTRHYRAPEVILELGWSQPCDVWSIGCIFIEYVLGFLVQPTHNSREHLAMERILGPVPSRMIRKTRKQKYFYRGRLDWDENTSAGRYVRENCKPLRRYLTSEAEDHHQLFDLIENMLEYEPAKRLTLGEALQHPFFACLRTEPPNTKLWDSSRDISR

SEQ ID NO: 40 - protein, 484 aa, Mus musculus
HRCSKYRSPEPDPYLTYRWKERRSDSREHEGRLRYPSRKEPPPRASSREDAPYRTRKHAHHCHKIRTRSCSSASSRSQQSSKRSSRGESRERAPYRTRKHAHHCHKRRTRSCSSASSRSQQSSKRSSRSVEDDKEGHLVCRIGSWLQERYEIVGNLGEGTFGKVVECLDHARGKSQVALKIIRNVGHYREAARLEINVLKKIKEKDKENKFLCVLMSDWNFHRGMICAVELLGKNTFEFLKENNFQPYPLPHVRHMAYQLCHALRFLHENQLTHTDLKPENILFVNSDEFETLPKEHKSCETKSVKDTSIRDAGSATYDFEHHSTTVIATRHYRPPEVILELGWAQPCDVWSIGCILFEYYRGFTLFQTHDSKEHLAMMEKILGPIPSHMIHRTRKQKYFYKGGLVWDENSSDGRYVKENCKPLKSYMLQDSLEHVQLFDLMRRMLEFDPAQRITLAEALLHPFFAGLTPEERSFHSSSRNPSR

SEQ ID NO: 41 - protein, 481 aa, Mus musculus
MRHSKRTHCPDWDSRESWGHESYSGSHKRKRRSHSSTQENRHCKPHHQFKDSDCHYLEARCLNERDYRDRRYIDEYRNDYCEGYVPRHYHRDVESTYRIHCSKSSVRSRRSSPKRKRNRPCASHQSHSKSHRRKRSRSIEDDEEGHLICQSGDVLRARYEIVDTLGEGAFGKVVECIDHGMDGLHVAVKIVKNVGGYREAARSEIQVLEHLNSTDPNSVFRCVQMLEWFDHHGHVCIVFELLGLSTYDFIKENSFLPFQIDHIRQMAYQICQSINFLHHNKLTHTDLKPENILFVKSDYVVKNPSMKRDERTILKPTDIKVVDFGSATYDDEHHSTLVSTRHYRAPEVILALGWSQPCDVWSIGCILIEYYLGFTVFQTHDSKEHLAMMERILGPIPAHMIQKTRKRKYFHHNQLDWDEHSSAGRYVRRRCKPLKEFMLCHDEEHEKLFDLVRRMLEYDPARRITLDEALQHPFFDLLKRK

(19) United States
(12) Patent Application Publication
(10) Pub. No.: US 2008/0051556 A1
(43) Pub. Date: Feb. 28, 2008

(54) Novel PTP-20, PCP-2, BDP1, CLK, and SIRP proteins and related products and methods

(75) Inventors: Axel Ullrich, Munchen (DE); Naohito Aoki, Munchen (DE); Yeong Woong Kim, Soosung-gu (KR); Hong Yang Wang, Shanghai (CN); Zhengjun Chen, Graefelfing (DE); Oliver Nayler, Martinsried (DE); Alexei Kharitonenkov, Carmel, IN (US)

Correspondence Address: FOLEY AND LARDNER LLP;SUITE 500, 3000 K STREET NW, WASHINGTON, DC, 20007, US

(73) Assignee: Max-Planck-Gesellschaft Zur Forderung Der Wissenschaften, E.V.

(21) Appl. No.: 11/153,918

(22) Filed: Jun. 16, 2005

Related U.S. Application Data
(63) Continuation of application No. 10/087,993, filed on Mar. 5, 2002, now abandoned.
Application No. 10/087,993 is a continuation of application No. 08/877,150, filed on Jun. 17, 1997, now Pat. No. 7,074,589.
(60) Provisional application No. 60/019,629, filed on Jun. 17, 1996.
(60) Provisional application No. 60/023,485, filed on Aug. 9, 1996.
(60) Provisional application No. 60/030,860, filed on Nov. 13, 1996.
(60) Provisional application No. 60/030,964, filed on Nov. 15, 1996.
(60) Provisional application No. 60/034,286, filed on Dec. 19, 1996.

(51) Int. Cl. C07K 2/00; C07H 21/00; C07K 16/00

(52) U.S. Cl. 530/300; 530/387.1; 536/23.5

(57) ABSTRACT

Nucleic acid molecules encoding full length PTP20, PCP-2, BDP1, mCLK2, mCLK3, mCLK4, and SIRP polypeptides, portions of such nucleic acid molecules, nucleic acid vectors containing such nucleic acid molecules, recombinant cells containing such nucleic acid vectors, polypeptides purified from such recombinant cells, antibodies to such polypeptides, and methods of identifying compounds that bind such polypeptides or abrogate their interactions with natural binding partners. Methods for diagnosing abnormal conditions in an organism with PTP20, PCP-2, BDP1, mCLK2, mCLK3, mCLK4, and SIRP related molecules or compounds. PTP20, PCP-2, BDP1, mCLK2, mCLK3, mCLK4, or SIRP polypeptides, nucleic acids encoding such polypeptides, cells, tissues and animals containing such nucleic acids, antibodies to such polypeptides, assays utilizing such polypeptides, and methods relating to all of the foregoing. Methods for treatment, diagnosis, and screening are provided for diseases related to PTP20, PCP-2, BDP1, mCLK2, mCLK3, mCLK4, and SIRP polypeptides or conditions characterized by an abnormal interaction between such a polypeptide and its binding partner.

### RELATED APPLICATIONS

This application claims priority to (1) Aoki, et al., PROTEIN TYROSINE PHOSPHATASE PTP20 AND RELATED PRODUCTS AND METHODS, U.S. Patent Application 60/019,629, filed Jun. 17, 1996; (2) Kim, et al., PROTEIN BDP1, U.S. Patent Application 60/023,485, filed Aug. 9, 1996; (3) Wang, et al., PROTEIN PCP-2, U.S. Patent Application 60/030,860, filed Nov. 13, 1996; (4) Naylor, et al., CLK PROTEIN KINASES AND RELATED PRODUCTS AND METHODS, U.S. Patent Application 60/034,286, filed Dec. 19, 1996; and, (5) Ullrich, et al., SIRP PROTEINS AND USES THEREOF, U.S. Patent Application 60/030,964, filed Nov. 15, 1996; all of which are hereby incorporated herein by reference in their entirety, including any drawings.

### INTRODUCTION

The present invention relates generally to newly identified proteins involved in cellular signal transduction including protein tyrosine phosphatases, protein serine/threonine kinases, downstream signaling molecules, and related products and methods. The novel proteins are called PTP20, BDP1, PCP-2, CLK, and SIRP.

### BACKGROUND OF THE INVENTION

The following description of the background of the invention is provided to aid in understanding the invention, but is not admitted to describe or constitute prior art to the invention.

Cellular signal transduction is a fundamental mechanism whereby external stimuli that regulate diverse cellular processes are relayed to the interior of cells. One of the key biochemical mechanisms of signal transduction involves the reversible phosphorylation of proteins, which enables regulation of the activity of mature proteins by altering their structure and function. Enzymes that mediate phosphorylation of cellular effectors fall into two classes. While protein phosphatases hydrolyze phosphate moieties from phosphoryl protein substrates, protein kinases transfer a phosphate moiety from adenosine triphosphate to protein substrates. The converse functions of protein kinases and protein phosphatases balance and regulate the flow of signals in signal transduction processes.

Kinases largely fall into two groups, those specific for phosphorylating serines and threonines (STKs), and those specific for phosphorylating tyrosines (TKs). The protein phosphatases can also be classified as being specific for either serine/threonine (STPs) or tyrosine (PTPs). The known enzymes, both kinase and phosphatases, can be divided into two groups—receptor and non-receptor type proteins. Most receptor-type protein tyrosine phosphatases (RPTPs) contain two conserved catalytic tyrosine phosphatase domains each of which encompasses a segment of 240 amino acid residues (Saito et al, Cell Growth and Diff., 2:59, 1991). The RPTPs can be subclassified further based upon the amino acid sequence diversity, of their extracellular domains (Saito, et al., supra; Krueger, et al., PNAS 89:7417, 1992).

Alignment of primary amino acid sequences of known phosphatases and kinases shows that their catalytic domains share common amino acid sequences with other enzymes in their respective classes. This observation has facilitated efforts of cloning protein phosphatases from multiple organisms and tissues. Probing cDNA libraries with polynucleotides complementary to cDNA encoding protein phosphatase consensus sequences has identified cDNAs resembling protein phosphatase or kinase sequences via the polymerase chain reaction (PCR). Some polypeptide molecules encoded by these cDNAs have enzymatic activity.

Tyrosine phosphatases can down-regulate the catalytic activity of protein kinases involved in cell proliferation and are therefore thought to be possible candidate anti-cancer proteins. In addition to their role in cellular proliferation, protein phosphatases are thought to be involved in cellular differentiation processes. Cell differentiation occurs in some cells upon nerve growth factor (NGF) or epidermal growth factor (EGF) stimulation. Cellular differentiation is characterized by rapid membrane ruffling, cell flattening, and increases in cell adhesion. Chao, Cell 68:995-997, 1992.

In view of the above, it can be seen that a need exists to identify additional proteins whose inappropriate activity may lead to cancer or other disorders so that pharmaceutical compounds for the treatment of those disorders might also be identified.

### SUMMARY OF THE INVENTION

The present invention relates to a group of novel proteins designated PTP20, PCP-2, BDP1, mCLK2, mCLK3, mCLK4, and SIRP1 and SIRP4 and related polypeptides, nucleic acids encoding such polypeptides, nucleic acid vectors harboring such nucleic acid molecules, cells containing such nucleic acids, antibodies to such polypeptides, assays utilizing such polypeptides, methods of identifying compounds that bind such polypeptides or abrogate their interactions with natural binding partners, and additional methods relating to all of the foregoing. Also disclosed are methods for diagnosing and treating specific abnormal conditions in an organism with such polypeptides related molecules or compounds. The nucleic acid molecules, nucleic acid vectors, recombinant cells, polypeptides, and antibodies may be produced using well known and standard techniques used currently in the art. Each of the new proteins is described briefly below.

PTP20—

The present invention is based in part upon the isolation and characterization of nucleic acid molecules encoding a novel protein phosphatase designated PTP20. PTP20 regulates growth factor stimulation of cellular differentiation. PTP20 is thought to be involved in cellular differentiation, as its over-expression in rat pheochromocytoma cells (PC12) causes increased rates of differentiation. Various treatments of neural cancers as well as neural damage are thus provided based on the discovery of PTP20 and its role in these disorders.

The open reading frame of the full-length PTP20 nucleic acid molecule encodes a protein of 453 amino acids with a predicted molecular weight of approximately 50 kDa. Hydropathy analysis (see Kyte and Doolittle, 1982, J. Mol. Bio. 157:105-132) indicates that PTP20 contains no hydrophobic segments appropriate for signal peptide or transmembrane domains and therefore PTP20 is most likely an intracellular protein. The transcripts corresponding to nearly the same size of the full length cDNA are detected in several rat tissues including brain, liver, lung, spleen, skeletal muscle, kidney, and testis.

The catalytic domain is located near the predicted amino terminus between amino acids 58 and 283. The catalytic domain of PTP20 may be homologous to the PTP-PEST-family phosphatases, such as human and rat PTP-PESTs and PEP-PTP. Takekawa et al., 1992, Biochem. Biophys. Res. Commun. 189:1223-1230; Yang et al., 1993, J. Biol. Chem. 268:6622-6628; Matthews et al., 1992, Mol. Cell. Biol. 12:2396-2405. Proline, glutamate, serine, and threonine residues (PEST) are enriched in the PEST-motif sequence, which is not arranged in any specified consensus sequence. Rechsteiner and Rogers, 1996, TIBS 21:267-271. PTP20 may have a PEST sequence between amino acids 285 and 453, suggesting that PTP20 may be a member of the PTP-PEST family.

Experimental results implicate PTP20 as an essential agent involved in a growth factor stimulated cellular differentiation signal transduction pathway. Although most cells have already differentiated in adults, activators of PTP20 might cause differentiation instead of proliferation of cellular tumors and therefore act as anti-cancer therapeutics. In addition, inhibitors of PTP20 might be useful for treating neural injuries by delaying the differentiation of transplanted neuronal stem cells until they are firmly grafted.

BDP1—

A second PTP of the invention is BDP-1 (Brain Derived Phosphatase 1). Like PTP20, BDP-1 has no transmembrane sequence and is likely, therefore, to be an intracellular protein. BDP-1 was originally identified in a human brain cDNA library, although the full length BDP1 clone was isolated from the hematopoietic MEGO1 cDNA library. The nucleotide sequence was found to be 2810 bp, and the open reading frame was 459 amino acids long. Northern hybridization showed a 2.8 Kb signal, corresponding to the length of the BDP1 clone. There is an ATG start codon at the 5′-end, a GC-rich sequence downstream from the start codon, a poly(A)+tail, with a polyadenylation signal and a T-rich sequence at the 3′-noncoding sequence.

BDP-1 is similar in sequence and structure to PTP20 (approximately 85% identity at the amino acid level). The predicted amino acid sequence shared about 36 to 38% homology with the PTPase-PEST family, which spanned only through the putative catalytic domain. The N-terminal sequence was homologous with the N-terminus of the cyclase-associated CAP protein. The last sequence with approximately 20 amino acids at the C-terminus was homologous with the PTPase-PEST family and the cytoplasmic tail sequence of MHC antigen I protein.

The tyrosine phosphatase activity of BDP1 and its expression were confirmed using p-nitrophenylphosphate and autophosphorylated proteins, such as src and several chimeric receptor proteins which were cotransfected into human kidney embryonic 293 cells with BDP1. BDP1 was expressed in most tissues and cell lines at basal level, but expressed high in epithelium origin cell lines and cancer cell lines.

PCP-2—

A third PTP of the invention is a novel receptor-type protein phosphatase, containing a MAM domain, designated PCP-2 (pancreatic carcinoma phosphatase 2). The MAM domain is a newly defined sequence motif that was identified in the functionally diverse receptors meprin, A5 protein, PTPk, and PTPm (Beckman G. and Bork P. Trends Biochem. Sci. 18:40, 1993; Jiang, et al. J. Biol. Chem. 267:9185, 1992; Tagaki, et al. Neuron. 7:295, 1991). At present, the function of this domain is not known although it may be involved in cell-cell interaction.

PCP-2 appears to be a transmembrane protein of 1430 amino acids, whose extracellular domain shares the structural motifs with mouse PTPk and human and mouse PTPm. A potential role of PCP-2 in cell-cell recognition and adhesion is supported by its co-localization with the cell adhesion molecules b-caternin and E-cadherin at sites of cell-cell contact.

CLKs—

CLK serine/threonine kinases regulate RNA splicing in cells and some are highly expressed in cancer cells as well as testis. The present invention discloses the discovery of the protein kinases, mCLK2, mCLK3, and mCLK4. The predicted molecular weights of the encoded proteins are 59.9 kDa (mCLK2), 58.5 kDa (mCLK3), and 57.2 kDa (mCLK4). Various mCLK2, mCLK3, and mCLK4 related molecules and compounds can now be designed as treatments of cancers or as contraceptives to reproduction in male organisms.

As illustrated in FIG. 1, mCLK1, mCLK2, mCLK3, and mCLK4 share the essential features identifying them as LAMMER kinases. (Yun et al., Genes. Dev. 8:1160, 1994.) They contain a nuclear localization signal (Dingwall and Laskey, Trends Biochem. Sci. 16:478, 1991), as well as an unusually basic amino terminus composed of many serine and arginine residues. These serine and arginine amino acids likely embody a signal sequence localizing the protein to nuclear speckles. (Hedley et al., PNAS 92:11524, 1995; Colwill et al., EMBO J. 15:265, 1996). The amino terminus is the most divergent portion of the proteins, suggesting that this area could contain information specific to each protein. The catalytic domain is homologous among all family members, with only few amino acid changes. Furthermore, all amino acids known to define the subfamily of CDC2 like kinases are present in all four proteins. (Ben-David et al., EMBO J. 10:317, 1991.)

mCLK1 has been shown to interact with ASF/SF2, SRp2O and hnRNP proteins in a yeast two hybrid system. Because hnRNP-K binds to the protooncogene p95^{vav}, mCLK1 could be implicated in transmitting signals that regulate the expression of the protooncogenes myc and fos in hematopoietic cells. Thus the role of CLK serine/threonine kinases may not be limited to simply maintaining RNA splicing and translocation events in the cell; CLK serine/threonine kinases may also be linked to regulating the flow of extracellular signals within hematopoietic cells. In addition, CLK serine/threonine kinases may be targets for compounds that could ameliorate cancers associated with uncontrolled regulation of the protooncogenes p95^{vav}, myc, and fos. Because over-expression of CLK serine/threonine kinases themselves have been implicated in certain types of cancer cell lines, compounds that inhibit their catalytic activity or disrupt their interactions with natural binding partners may act as anti-cancer therapeutics.

Even though CLK serine/threonine kinases other than mCLK2, mCLK3, and mCLK4 have been described previously, the methods of the invention relate to CLK serine/threonine kinases in general as the methods described herein are not disclosed elsewhere. Thus the methods of the invention include antibodies and other compounds with specific binding affinity to mCLK2, mCLK3, and mCLK4 as well as antibodies and other compounds that interact with other CLK protein kinase polypeptides.

SIRP Proteins—

The invention also encompasses a family of proteins that appear to be involved in the regulation of PTP activity, the SIRPs (SIgnal Regulatory Proteins). This family contains at least fifteen members that fall into two subtypes. All SIRP proteins have a receptor-like, or Immunoglobulin (Ig) like extracellular domain and a transmembrane domain. The two subtypes of SIRPs are distinguished by the presence or absence of a cytoplasmic SHP-2 binding domain. For example, SIRP4 has a cytoplasmic domain while SIRP1 does not. The cytoplasmic domain of SIRP4 contains two SHP-2 binding regions each having two tyrosine residues. SHP-2 is a tyrosine phosphatase well known to be involved in cellular signal transduction. It has two SH2 domains and is required for signaling downstream of a variety of RTKs. SHP-2 has been reported to bind directly to RTKs such as PDGF receptor, EGF receptor, and cKit in response to stimulation by their ligands. Insulin receptor substrate 1 (IRS-1) also associates with SHP-2 in response to insulin.

SIRP4 has negative regulatory effects on growth factor and hormone induced cellular responses. This effect depends on phosphorylation of SIRP4 tyrosines and is related to reduced MAP kinase activation. SIRP4 becomes a substrate of activated receptor tyrosine kinases (RTKs) upon EGF, insulin or PDGF stimulation. In its tyrosine phosphorylated form, SIRP4 binds a phosphotyrosine phosphatase, SHP-2, via SH2 interactions. Once SIRP4 binds SHP-2, it activates the catalytic activity of SHP-2 and becomes a substrate of SHP-2. This direct activation of SHP-2 could induce activation of Src or other Src family kinases. The above described interaction allows SIRP4 to participate in major signal transduction pathways involving SHP-2. SIRP4 also binds SHP-1 and Grb2, both of which contain a SH-2 domain. Grb2 is an adapter molecule and one of its functions is to link growth factor receptors to downstream effector proteins. Grb2 is known to bind tyrosine-phosphorylated SHP-2 in response to PDGF stimulation.

SIRP family proteins play a general role in the regulation of signals that define diverse physiological and pathological processes. In particular, SIRP polypeptides are involved in various signal transduction pathways such as the negative regulation of signals generated by receptor tyrosine kinases, including, but not limited to, receptors for EGF, insulin and platelet derived growth factor (PDGF). For example, acting like a tumor suppressor, SIRP4 exerts negative regulatory effects on growth factor and hormone induced cellular responses such as DNA synthesis. Oncogenesis may be associated with mutant SIRPs or not enough SIRPs. Restoring SIRPs to their normal levels such as by gene therapy could restore the cells to a normal growth pattern. Insulin receptor activity is also regulated by SIRPs. Overexpression of SIRPs may be involved in type II diabetes where sufficient insulin is present but insulin signaling is deficient. compound that inhibits the negative regulation of insulin signaling by SIRPS, such as by interfering with the interaction between SIRP and SHP-2 may lead to enhanced insulin signaling.

Isolated Nucleic Acids

Thus in a first aspect, the invention features an isolated, enriched, or purified nucleic acid molecule encoding a PTP20, PCP-2, BDP1, mCLK2, mCLK3, mCLK4, or SIRP polypeptide.

By “isolated” in reference to nucleic acid is meant a polymer of 6 (preferably 21, more preferably 39, most preferably 75) or more nucleotides conjugated to each other, including DNA or RNA that is isolated from a natural source or that is synthesized. In certain embodiments of the invention longer nucleic acids are preferred, for example those of 300, 600, 900 or more nucleotides and/or those having at least 50%, 60%, 75%, 90%, 95% or 99% “identity” to the full length sequence shown in FIG. 1, 2, 3, 4, or 5 respectively for PTP20, PCP2, BDP1, mCLK2, mCLK3, mCLK4, or SIRP polypeptide.

By “identity” is meant a property of sequences that measures their similarity or relationship. Identity is measured by dividing the number of identical residues by the total number of residues and multiplying the product by 100. Thus, two copies of exactly the same sequence have 100% identity, but sequences that are less highly conserved and have deletions, additions, or replacements may have a lower degree of identity. Those skilled in the art will recognize that several computer programs are available for determining sequence identity.

The isolated nucleic acid of the present invention is unique in the sense that it is not found in a pure or Separated state in nature. Use of the term “isolated” indicates, that a naturally occurring sequence has been removed from its normal cellular (i.e., chromosomal) environment. Thus, the sequence may be in a cell-free solution or placed in a different cellular environment. The term does not imply that the sequence is the only nucleotide chain present, but that it is essentially free (about 90-95% pure at least) of non-nucleotide material naturally associated with it and thus is meant to be distinguished from isolated chromosomes.

The term “enriched” in reference to nucleic acid means that the specific DNA or RNA sequence constitutes a significantly higher fraction (2-5 fold) of the total DNA or RNA present in the cells or solution of interest than in normal or diseased cells or in the cells from which the sequence was taken. This could be caused by a person skilled in the art by preferential reduction in the amount of other DNA or RNA present, or by a preferential increase in the amount of the specific DNA or RNA sequence, or by a combination of the two. However, enriched does not imply that there are no other DNA or RNA sequences present, just that the relative amount of the sequence of interest has been “significantly increased,” in a useful manner and prefer. The term “significantly” qualifies “increased” to indicate that the level of increase is useful to the person performing the recombinant DNA technique, and generally means an increase relative to other nucleic acids of about at least 2 fold, more preferably at least 5 to 10 fold or even more. The term also does not imply that there is no DNA or RNA from other sources. The other source DNA may, for example, comprise DNA from a yeast or bacterial genome, or a cloning vector such as pUC19. This term distinguishes from naturally occurring events, such as viral infection, or tumor type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level this level should be at least 2-5 fold greater, e.g., in terms of mg/ml). Individual clones isolated from a cDNA library may be purified to electrophoretic homogeneity. The claimed DNA molecules obtained from these clones could be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 106-fold purification of the native message. Thus, purification of at least one order of magnitude, preferably two or three orders, and more preferably four or five orders of magnitude is expressly contemplated.

The term “PTP20 polypeptide” refers to a polypeptide having an amino acid sequence preferably of at least 400 contiguous amino acids, more preferably of at least 450 contiguous amino acids, or most preferably of at least 453 contiguous amino acids set forth in FIG. 1, or is substantially similar to such a sequence. A sequence that is substantially similar will preferably have at least 90% identity (more preferably at least 95% and most preferably 99-100%) identity to the amino acid sequence of FIG. 1. PTP20 polypeptides preferably have tyrosine phosphatase activity and fragments of the full length PTP20 sequence having such activity may be identified using techniques well known in the art, such as sequence comparisons and assays such as those described in the examples herein.

By “a PCP-2 polypeptide” or a “BDP1 polypeptide” is meant 25 (preferably 30, more preferably 35, most preferably 40) or more contiguous amino acids set forth in the full length amino acid sequence of FIG. 2 or 3, respectively, or a functional derivative thereof as described herein. In certain aspects, polypeptides of 100, 200, 300 or more are preferred. The PCP-2 or the BDP1 polypeptide can be encoded by a full-length nucleic acid sequence or any portion of the full-length nucleic acid sequence, so long as a functional activity of the polypeptide is retained.

The terms “mCLK2”, “mCLK3”, and “mCLK4” refer to polypeptides that have amino acid sequences substantially similar to those set forth in FIG. 4. A sequence that is substantially similar will preferably have at least 95% identity, more preferably at least 96-97% identity, and most preferably 98-100% identity to the sequence of FIG. 4. CLK protein kinase polypeptides preferably have protein kinase activity and fragments of the full length CLK protein kinase sequences having such activity may be identified using techniques well known in the art, such as sequence comparisons and assays such as those described in the examples herein.

By “SIRP polypeptide” is meant 9 or more contiguous amino acids set forth in the full length amino acid sequence of FIG. 5. The SIRP polypeptides can be encoded by full-length nucleic acid sequences or any portion of a full-length nucleic acid sequence, so long as a functional activity of the polypeptide is retained. Preferred functional activities include the ability to bind to a receptor tyrosine kinase or a SH-2 domain bearing protein such as SHP-2, SHP-1 or Grb-2. A non full-length SIRP polypeptide may be used to elicit an antibody against the polypeptide and the full-length polypeptide using techniques known to those skilled in the art. The present invention also encompasses deletion mutants lacking one or more isolated SIRP domains (e.g., Ig-like domain, transmembrane domain, SH2 binding domain, and tyrosine residues), and complementary sequences capable of hybridizing to full length SIRP protein under stringent hybridization conditions.

A preferred embodiment concerns an isolated nucleic acid molecule relating to PTP20 that encodes at least twelve contiguous amino acids of the amino acid sequence set forth in FIG. 1. Preferably at least 12, 15, 20, 25, 30, 35, 40, 50, 100, 200 or 300 contiguous amino acids or the PTP20 sequence are encoded. In another preferred embodiment the isolated nucleic acid comprises, consists essentially of, or consists of a nucleic acid sequence, which encodes a PCP-2 or BDP1 polypeptide, set forth in the full length amino acid sequence of FIG. 2 or 3, respectively, a functional derivative thereof, or encodes at least 25, 30, 35, 40, 50, 100, 200 or 300 contiguous amino acids thereof. Another preferred embodiment of the invention concerns isolated nucleic acid molecules that encode at least seventeen amino acids of a mCLK2, mCLK3, or mCLK4 polypeptide. Preferably, at least 17, 20, 25, 30, 35, 40, 50, 100, 200, 300, 400, 450, 475, or 485 contiguous amino acids are encoded. In other preferred embodiments, isolated nucleic acid comprises, consists essentially of, or consists of a nucleic acid sequence, which encodes a SIRP polypeptide, set forth in the full length amino acid sequence of FIG. 5, or a functional derivative thereof, or at least 25, 30, 35, 40, 50, 100, 200 or 300 contiguous amino acids thereof. These preferred embodiments of the invention are achieved by applying routine recombinant DNA techniques known to those skilled in the art.

By “comprising” it is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The nucleic acid may be isolated from a natural source by cDNA cloning or subtractive hybridization; the natural source may be mammalian (human) blood, semen, or tissue of various organisms including eukaryotes, mammals, birds, fish, plants, gorillas, rhesus monkeys, chimpanzees and humans. The nucleic acid may be synthesized by the triester method or by using an automated DNA synthesizer. In other preferred embodiments the isolated nucleic acid may be at least 95% identical to the nucleic acid sequence shown in FIG. 1, 2, 3, 4, or 5 and is capable of hybridizing to the nucleic acid sequence shown in FIG. 1, 2, 3, 4, or 5, preferably under stringent hybridization conditions.

In yet other preferred embodiments the nucleic acid is a conserved or unique region, for example those useful for the design of hybridization probes to facilitate identification and cloning of additional polypeptides, the design of PCR probes to facilitate cloning of additional polypeptides, and obtaining antibodies to polypeptide regions. Examples of amino acid sequences of the present invention include the following amino acid sequences (the isolated, purified or enriched nucleic acids encoding them are also within the scope of the present invention).

The term “hybridize” refers to a method of interacting a nucleic acid probe with a DNA or RNA molecule in solution or on a solid support, such as cellulose or nitrocellulose. If a nucleic acid probe binds to the DNA or RNA molecule with high affinity, it is said to “hybridize” to the DNA or RNA molecule. As mentioned above, the strength of the interaction between the probe and its target can be assessed by varying the stringency of the hybridization conditions. Various low or high stringency hybridization conditions may be used depending upon the specificity and selectivity desired. Stringency is controlled by varying salt or denaturant concentrations. Under stringent hybridization conditions only highly complementary nucleic acid sequences hybridize. Preferably, such conditions prevent hybridization of nucleic acids having one or two mismatches out of 20 contiguous nucleotides. Examples of various hybridization conditions are shown in the examples below.

By “conserved nucleic acid regions”, are meant regions present on two or more nucleic acids encoding a PTP20, PCP-2, BDP1, CLK protein kinase, or SIRP polypeptide, to which a particular nucleic acid sequence can hybridize under lower stringency conditions. Examples of lower stringency conditions suitable for screening for nucleic acid encoding PTP20, PCP-2, BDP1, CLK protein kinase, or SIRP polypeptides are provided in Abe, et al. J. Biol. Chem., 19:13361 (1992) (hereby incorporated by reference herein in its entirety, including any drawings). Preferably, conserved regions differ by no more than 5 or 7 out of 20 nucleotides, preferably differ by no more than 5 out of 20 nucleotides, more preferably differ by no more than 10 out of 20 nucleotides, and most preferably differ by no more than 15 out of 20 nucleotides. Protein kinases share conserved regions in the catalytic domain.

By “unique nucleic acid region” is meant a sequence present in a full length nucleic acid coding for a PTP20, PCP-2, BDP1, CLK protein kinase, or SIRP polypeptide that is not present in a sequence coding for any other naturally occurring polypeptide. Such regions preferably comprise 30 or 45 contiguous nucleotides present in the full length nucleic acid encoding a PTP20, PCP-2, CLK protein kinase, or BDP1 polypeptide more preferably 100 contiguous nucleotides, and most preferably 200 contiguous nucleotides, or comprise 12 or 20 contiguous nucleotides present in the full length nucleic acid encoding a SIRP polypeptide. In particular, a unique nucleic acid region is preferably of mammalian origin.

Nucleic Acid Probes

Another aspect of the invention features a nucleic acid probe that can detect nucleic acid molecules encoding a PTP20, PCP-2, BDP1, mCLK2, mCLK3, mCLK4, or SIRP polypeptide in a sample.

The term “nucleic acid probe” refers to a nucleic acid molecule that is complementary to and can bind a nucleic acid sequence encoding an amino acid sequence substantially similar to that set forth in FIG. 1, 2, 3, 4, or 5.

Thus, the nucleic acid probe contains nucleic acid that will hybridize to a sequence set forth in FIG. 1, 2, 3, 4, or 5, or a functional derivative thereof.

In preferred embodiments the nucleic acid probe hybridizes to nucleic acid encoding at least 12, 75, 90, 105, 120, 150, 200, 250, 300 or 350 contiguous amino acids of the full-length sequence set forth in FIGS. 1-3, at least 17, 20, 25, 30, 35, 40, 50, 100, 200, 300, 400, 450, 475, or 485 contiguous amino acids of the full-length sequence set forth in FIG. 4, or at least 12, 27, 30, 35, 40, 50, 100, 200, or 300 contiguous amino acids of the full-length sequence set forth in FIG. 5, or a functional derivative thereof. Various low or high stringency hybridization conditions may be used depending upon the specificity and selectivity desired.

The nucleic acid probe can be labeled with a reporter molecule or molecules. The term “reporter molecule” refers to a molecule that is conjugated to the nucleic acid probe or is contained within the nucleic acid probe. The reporter molecule allows the detection of the probe by methods used in the art. Reporter molecules are chosen from, but not limited to, the group consisting of an enzyme, such as a peroxidase, a radioactive element, or an avidin or biotin molecule.

By “high stringency hybridization conditions” is meant those hybridizing conditions that (1) employ low ionic strength and high temperature for washing, for example, 0.015 M NaCl/0.0015 M sodium citrate/0.1% SDS at 50% C; (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42% C; or (3) employ 50% formamide, 5×SSC (0.75 M NaCl, 0.075 M Sodium pyrophosphate, 5×Denhardt's solution, sonicated salmon sperm DNA (50 g/ml), 0.1% SDS, and 10% dextran sulfate at 42% C, with washes at 42% C in 0.2×SSC and 0.1% SDS. Under stringent hybridization conditions only highly complementary nucleic acid sequences hybridize. Preferably, such conditions prevent-hybridization of nucleic acids having 1 or 2 mismatches out of 20 contiguous nucleotides, more preferably having 1 mismatch out of 35 contiguous nucleotides, and most preferably having 1 mismatch out of 50 contiguous nucleotides.

Methods for using the probes include detecting the presence or amount of PTP20, PCP-2, BDP1, CLK protein kinase, or SIRP RNA in a sample by contacting the sample with a nucleic acid probe under conditions such that hybridization occurs and detecting the presence or amount of the probe bound to such RNA. The nucleic acid duplex formed between the probe and a nucleic acid sequence coding for a PCP-2, SIRP, CLK protein kinase polypeptide may be used in the identification of the sequence of the nucleic acid detected (for example see, Nelson et al., in Nonisotopic DNA Probe Techniques, p. 275 Academic Press, San Diego (Kricka, ed., 1992) hereby incorporated by reference herein in its entirety, including any drawings). Kits for performing such methods may be constructed to include a container means having disposed therein a nucleic acid probe.

Nucleic Acid Vectors

In yet another aspect, the invention relates to a nucleic acid vector comprising a promoter element and a nucleic acid molecule described in this invention.

The term “nucleic acid vector” relates to a single or double stranded circular nucleic acid molecule that can be transfected or transformed into cells and replicate independently or within a cell genome. A vector can be cut and thereby linearized upon treatment with restriction enzymes. An assortment of vectors, restriction enzymes, and the knowledge of the nucleotide sequences that the restriction enzymes operate upon are readily available to those skilled in the art. A nucleic acid molecule encoding a PTP20, PCP-2, BDP1, CLK protein kinase, or SIRP polypeptide can be inserted into a vector by cutting the vector with restriction enzymes and ligating the two pieces together.

The term “promoter element” describes a nucleotide sequence that is incorporated into a vector that, once inside an appropriate cell, may facilitate transcription factor and/or polymerase binding and subsequent transcription of portions of the vector DNA into mRNA. The promoter element precedes the SI end of the nucleic acid molecule of the first aspect of the invention such that the latter is transcribed into mRNA. Recombinant cell machinery then translates mRNA into a polypeptide.

Many techniques are available to those skilled in the art to facilitate transformation or transfection of the nucleic acid vector into a prokaryotic or eukaryotic organism. The terms “transformation” and “transfection” refer to methods of inserting a nucleic acid vector into a cellular organism. These methods involve a variety of techniques, such as treating the cells with high concentrations of salt, an electric field, or detergent, to render the cell outer membrane or wall permeable to nucleic acid molecules of interest.

Recombinant Cells

The invention also features recombinant nucleic acid, preferably in a cell or an organism. The recombinant nucleic acid may contain a sequence set forth in FIGS. 1-5, or a functional derivative thereof and a vector or a promoter effective to initiate transcription in a host cell. The recombinant nucleic acid can alternatively contain a transcriptional initiation region functional in a cell, a sequence complimentary to an RNA sequence encoding a PTP20, PCP-2, BDP1, mCLK2, mCLK3, mCLK4, or SIRP polypeptide and a transcriptional termination region functional in a cell. The term “recombinant” refers to an organism that has a new combination of genes or nucleic acid molecules. A new combination of genes or nucleic acid molecules can be introduced to an organism using a wide array of nucleic acid manipulation techniques available to those skilled in the art.

In another aspect, the invention describes a recombinant cell or tissue containing a purified nucleic acid coding for a PTP20, PCP-2, BDP1, mCLK2, mCLK3, mCLK4, or SIRP polypeptide. In such cells, the nucleic acid may be under the control of its genomic regulatory elements, or may be under the control of exogenous regulatory elements including an exogenous promoter. By “exogenous” it is meant a promoter that is not normally coupled in vivo transcriptionally to the coding sequence for the PTP20, PCP-2, BDP1, mCLK2, mCLK3, mCLK4, or SIRP polypeptide.

The recombinant cell can be a eukaryotic or prokaryotic organism. The term “eukaryote” refers to an organism comprised of cells containing a nucleus. Eukaryotes are differentiated from “prokaryotes” which do not house their genomic DNA inside a nucleus. Prokaryotes include unicellular organisms such as bacteria while eukaryotes are represented by yeast, invertebrates, and vertebrates.

The recombinant cell can also harbor a nucleic acid vector that is extragenomic. The term “extragenomic” refers to a nucleic acid vector which does not integrate into a cell genome. Many nucleic acid vectors are designed with their own origins of replication which allow them to utilize the recombinant cell replication machinery to copy and propagate the nucleic acid vector nucleic acid sequence. These nucleic acid vectors are small enough that they are not likely to harbor nucleic acid sequences homologous to genomic sequences of the recombinant cell. Thus these nucleic acid vectors replicate independently of the genome and do not recombine with or integrate into the genome.

A recombinant cell can also harbor a portion of a nucleic acid vector in an intragenomic fashion. The term “intragenomic” defines a nucleic acid vector that integrates within a cell genome. Multiple nucleic acid vectors available to those skilled in the art contain nucleic acid sequences that are homologous to nucleic acid sequences in a particular organism's genomic DNA. These homologous sequences will result in recombination events that incorporate portions of the nucleic acid vector into the genomic DNA. Those skilled in the art can control which nucleic acid sequences of the nucleic acid vector integrate into the cell genome by flanking the portion to be integrated into the genome with homologous sequences in the nucleic acid vector.

Isolated Polypeptides

In another aspect the invention features an isolated, enriched, or purified PTP20, PCP-2, BDP1, mCLK2, mCLK3, mCLK4, or SIRP polypeptide.

By “isolated” in reference to a polypeptide is meant a polymer of 2 (preferably 7, more preferably 13, most preferably 25) or more amino acids conjugated to each other, including polypeptides that are isolated from a natural source or that are synthesized. In certain aspects longer polypeptides are preferred, such as those with 402, 407, 413, or 425 contiguous amino acids of PCP-2 set forth in FIG. 2, those with 400, 450, 475, or 485 of the contiguous amino acids of mCLK2, mCLK3, or mCLK4 set forth in FIG. 4. The isolated polypeptides of the present invention are unique in the sense that they are not found in a pure or separated state in nature. Use of the term “isolated” indicates that a naturally occurring sequence has been removed from its normal cellular environment. Thus, the sequence may be in a cell-free solution or placed in a different cellular environment. The term does not imply that the sequence is the only amino acid chain present, but that it is essentially free (about 90-95% pure at least) of non-amino acid material naturally associated with it.

By the use of the term “enriched” in reference to a polypeptide is meant that the specific amino acid sequence constitutes a significantly higher fraction (2-5 fold) of the total of amino acids present in the cells or solution of interest than in normal or diseased cells or in the cells from which the sequence was taken. This could be caused by a person skilled in the art by preferential reduction in the amount of other amino acids present, or by a preferential increase in the amount of the specific amino acid sequence of interest, or by a combination of the two. However, enriched does not imply that there are no other amino acid sequences present, just that the relative amount of the sequence of interest has been significantly increased. The term significant here is used to indicate that the level of increase is useful to the person making such an increase, and generally means an increase relative to other amino acids of about at least 2 fold, more preferably at least 5 to 10 fold or even more. The term also does not imply that there is no amino acid from other sources. The other source amino acid may, for example, comprise amino acid encoded by a yeast or bacterial genome, or a cloning vector such as pUC19. The term is meant to cover only those situations in which a person has intervened to elevate the proportion of the desired nucleic acid.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment (compared to the natural level this level should be at least 2-5 fold greater, e.g., in terms of mg/ml). Purification of at least one order of magnitude, preferably two or three orders, and more preferably four or five orders of magnitude is expressly contemplated. The substance is preferably free of contamination at a functionally significant level, for example 90%, 95%, or 99% pure.

In preferred embodiments, the PTP20 polypeptide contains at least 12, 15, 20, 25, 30, 35, 40, 50, 100, 150, 200, 250, 300, or 350 contiguous amino acids of the full-length amino acid sequence of PTP20 set forth in FIG. 1, the PCP-2 or BDP1 polypeptide contains at least 25, 30, 35, 40, 50, 100, 150, 200, 250, 300, or 350 contiguous amino acids of the full-length sequence set forth in FIGS. 2 and 3, respectively, the mCLK2, mCLK3, or mCLK4 polypeptide contains at least 17, 20, 25, 30, 35, 40, 50, 100, 200, 300, 400, 450, 475, or 485 contiguous amino acids of a mCLK2, mCLK3, or mCLK4 polypeptide set forth in FIG. 4, or the SIRP polypeptide contains at least 9, 10, 15, 20, or 30 contiguous amino acids of the full-length sequence set forth in FIG. 5, or a functional derivative thereof.

Recombinant Polypeptides

In another aspect, the invention describes a recombinant polypeptide comprising a PTP20, PCP-2, BDP1, mCLK2, mCLK3, mCLK4, or SIRP polypeptide or a unique fragment thereof. By “unique fragment,” is meant an amino acid sequence present in a full-length PTP20, PCP-2, BDP1, or SIRP, or minimum stretch of amino acids in one mCLK molecule that is different in sequence than any other portion of another protein kinase or polypeptide that is not present in any other naturally occurring polypeptide. Preferably, such a sequence comprises 6 contiguous amino acids, more preferably 12 contiguous amino acids, even more preferably 18 contiguous amino acids present in the full sequence. For example, since the largest identical stretch of amino acids found in FIG. 4 is seventeen amino acids, the minimum unique fragment for a mCLK protein kinase is seventeen amino acids.

By “recombinant PTP20 polypeptide”, “recombinant PCP-2 polypeptide”, “recombinant BDP1 polypeptide”, “recombinant mCLK2 polypeptide”, “recombinant mCLK3 polypeptide”, “recombinant mCLK4 polypeptide”, or “recombinant SIRP polypeptide” is meant to include a polypeptide produced by recombinant DNA techniques such that it is distinct from a naturally occurring polypeptide either in its location (e.g., present in a different cell or tissue than found in nature), purity or structure. Generally, such a recombinant polypeptide will be present in a cell in an amount different from that normally observed in nature.

Antibodies

In another aspect, the invention features a PTP20, PCP-2, BDP1, mCLK2, mCLK3, mCLK4, or SIRP polypeptide binding agent able to bind to the polypeptide. The binding agent is preferably a purified antibody (e.g., a monoclonal or polyclonal antibody) having specific binding affinity to a PTP20, PCP-2, BDP1, mCLK2, mCLK3, mCLK4, or SIRP polypeptide. The antibody contains a sequence of amino acids that recognizes an epitope present on a PTP20, PCP-2, BDP1, mCLK2, mCLK3, mCLK4, or SIRP polypeptide. Other binding agents include molecules which bind to the PTP20, PCP-2, BDP1, mCLK2, mCLK3, mCLK4, or SIRP polypeptide and analogous molecules which bind to the polypeptide. Such binding agents may be identified by using assays that measure PTP20, PCP-2, BDP1, mCLK2, mCLK3, mCLK4, or SIRP binding partner activity.

By “purified” in reference to an antibody is meant that the antibody is distinct from naturally occurring antibody, such as in a purified form. Preferably, the antibody is provided as a homogeneous preparation by standard techniques. Uses of antibodies to the cloned polypeptide include those to be used as therapeutics, or as diagnostic tools.

By “specific binding affinity” is meant that the antibody binds to PTP20, PCP-2, BDP1, mCLK2, mCLK3, mCLK4, or SIRP polypeptide with greater affinity than it binds to other polypeptides under specified conditions. The present invention also encompasses antibodies that can distinguish hSIRP1 from hSIRP2 or hSIRP3 or can otherwise distinguish between the various SIRPs.

The term “polyclonal” refers to a mixture of antibodies with specific binding affinity to a PTP20, PCP-2, BDP1, mCLK2, mCLK3, mCLK4, or SIRP polypeptide, while the term “monoclonal” refers to one type of antibody with specific binding affinity to such polypeptide. Although a monoclonal antibody binds to one specific region on a PTP20 polypeptide, a polyclonal mixture of antibodies can bind multiple regions of a PTP20, PCP-2, BDP1, mCLK2, mCLK3, mCLK4, or SIRP polypeptide.

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target.

Antibodies having specific binding affinity to a PTP20, PCP-2, BDP1, mCLK2, mCLK3, mCLK4, or SIRP polypeptide may be used in methods for detecting the presence and/or amount of the polypeptide in a sample by contacting the sample with the antibody under conditions such that an immunocomplex forms and detecting the presence and/or amount of the antibody conjugated to the PTP20, PCP-2, BDP1, mCLK2, mCLK3, mCLK4, or SIRP polypeptide. Diagnostic kits for performing such methods may be constructed to include a first container means containing the antibody and a second container means having a conjugate of a binding partner of the antibody and a label.

Hybridoma

In another aspect the invention features a hybridoma which produces an antibody having specific binding affinity to a PTP20, PCP-2, BDP1, mCLK2, mCLK3, mCLK4, or SIRP polypeptide. By “hybridoma” is meant an immortalized cell line which is capable of secreting an antibody, for example a PTP20, PCP-2, BDP1, mCLK2, mCLK3, mCLK4, or SIRP antibody. In preferred embodiments the PTP20, PCP-2, BDP1, mCLK2, mCLK3, mCLK4, or SIRP antibody comprises a sequence of amino acids that is able to specifically bind to the said polypeptide.

Deletion Mutants

In another aspect, the invention provides a nucleic acid molecule comprising a nucleotide sequence that encodes a polypeptide having the full length amino acid sequence set forth in FIG. 1, 2, 3, 4, or 5 except that it lacks at least one domain selected from the group consisting of the N-terminal, catalytic, or C terminal domains. Such deletion mutants are useful in the design of assays for protein inhibitors. The nucleic acid molecules described above may be, for example, cDNA or genomic DNA and may be placed in a recombinant vector or expression vector. In such a vector, the nucleic acid preferably is operatively associated with the regulatory nucleotide sequence containing transcriptional and translational regulatory information that controls expression of the nucleotide sequence in a host cell.

The term “domain” refers to a region of a polypeptide which contains a particular function. For instance, N-terminal or C terminal domains of signal transduction proteins can serve functions including, but not limited to, binding molecules that localize the signal transduction molecule to different regions of the cell or binding other signaling molecules directly responsible for propagating a particular cellular signal. Some domains can be expressed separately from the rest of the protein and function by themselves, while others must remain part of the intact protein to retain function. The latter are termed functional regions of proteins and also relate to domains.

The term “N-terminal domain” refers to a portion of the full length amino acid sequences spanning from the amino terminus to the start of the catalytic domain.

The term “catalytic domain” refers to a portion of the full length amino acid molecules that does not contain the N-terminal domain or C-terminal region and has catalytic activity.

The term “C-terminal region” refers to a portion of the full length amino acid molecules that begins at the end of the catalytic domain and ends at the carboxy terminal amino acid, which is the last amino acid encoded before the stop codon in the nucleic acid sequence.

Functional regions of the PTP20, PCP-2, BDP1, mCLK2, mCLK3, mCLK4, or SIRP polypeptides may be identified by aligning their amino acid sequences with amino acid sequences of other polypeptides with known functional regions. If regions of the PTP20, PCP-2, BDP1, mCLK2, mCLK3, mCLK4, or SIRP polypeptide share high amino acid identity with the amino acid sequences of known functional regions, then the polypeptides can be determined to contain these functional regions by those skilled in the art. The functional regions can be determined, for example, by using computer programs and sequence information available to those skilled in the art.

Other functional regions of signal transduction molecules that may exist within PTP20, PCP-2, BDP1, mCLK2, mCLK3, mCLK4, or SIRP include, but are not limited to, proline-rich regions or phosphoryl tyrosine regions. These regions can interact with natural binding partners such as SH2 or SH3 domains of other signal transduction molecules.

Thus, the invention also provides a genetically engineered host cell containing any of the nucleotide sequences described herein and the nucleic acid preferably is operatively associated with the regulatory nucleotide sequence containing transcriptional and translational regulatory information that controls expression of the nucleotide sequence in a host cell. Such host cells may obviously be either prokaryotic or eukaryotic.

Detecting Binding Partners

Another aspect of the invention features a method of detecting the presence or amount of a compound capable of binding to a PTP20, PCP-2, BDP1, mCLK2, mCLK3, mCLK4, or SIRP polypeptide. The method involves incubating the compound with a PTP20, PCP-2, BDP1, mCLK2, mCLK3, mCLK4, or SIRP polypeptide and detecting the presence or amount of the compound bound to the polypeptide.

The term “natural binding partners” refers to polypeptides that bind to PTP20, PCP-2, BDP1, CLK protein kinase, or SIRP peptides and play a role in propagating a signal in a signal transduction process. The term “natural binding partner” also refers to a polypeptide that binds to PTP20, PCP-2, BDP1, CLK protein kinase, or SIRP peptides within a cellular environment with high affinity. High affinity represents an equilibrium binding constant on the order of 10-1 M. However, a natural binding partner can also transiently interact with a PTP20, PCP-2, BDP1, CLK protein kinase, or SIRP peptides and chemically modify it. Natural binding partners of such peptides are chosen from a group consisting of, but not limited to, src homology 2 (SH2) or 3 (SH3) domains, other phosphoryl tyrosine binding domains, and receptor and non-receptor protein kinases or protein phosphatases.

Methods are readily available in the art for identifying binding partners of polypeptides of interest. These methods include screening cDNA libraries included in one nucleic acid vector with a nucleic acid molecule encoding the desired polypeptide in another nucleic acid vector. Vojtek et al., 1993, Cell 74:205214. These techniques often utilize yeast recombinant cells. These techniques also utilize two halves of a transcription factor, one half that is fused to a polypeptide encoded by the cDNA library and the other that is fused to the polypeptide of interest. Interactions between a polypeptide encoded by the cDNA library and the polypeptide of interest are detected when their interaction concomitantly brings together the two halves into an active transcription factor which in turn activates a gene that reports the interaction. Any of the nucleic molecules encoding PTP20, PCP-2, BDP1, mCLK2, mCLK3, mCLK4, or SIRP peptides can be readily incorporated into an nucleic acid vector used in such a screening procedure by utilizing standard recombinant DNA techniques in the art.

Change in Activity

In yet another aspect, the invention relates to a method of identifying compounds capable of inhibiting or activating the PTP20, PCP-2, BDP1, mCLK2, mCLK3, mCLK4, or SIRP phosphorylation activity. This method comprises the following steps: (a) adding a compound to a mixture comprising a PTP20, PCP-2, BDP1, mCLK2, mCLK3, mCLK4, or SIRP polypeptide and a substrate for the polypeptide; and (b) detecting a change in phosphorylation of said substrate.

The term “compound” includes small organic molecules including, but not limited to, oxindolinones, quinazolines, tyrphostins, quinoxalines, and extracts from natural sources.

The term “a change in phosphorylation”, in the context of the invention, defines a method of observing a change in phosphorylation of the substrate in response to adding a compound to cells. The phosphorylation can be detected, for example, by measuring the amount of a substrate that is converted to a product with respect to time. Addition of a compound to cells expressing a PTP20, PCP-2, BDP1, mCLK2, mCLK3, mCLK4, or SIRP polypeptide may either enhance (activate) or lower (inhibit) the phosphorylation. If a compound lowers phosphorylation, the compound is assumed to bind to a PTP20, PCP-2, BDP1, mCLK2, mCLK3, mCLK4, or SIRP polypeptide and block the ability of CLK protein kinase to bind and/or turn over a substrate. If a compound enhances phosphorylation, the compound is assumed to bind to a PTP20, PCP-2, BDP1, mCLK2, mCLK3, mCLK4, or SIRP polypeptide and facilitate the ability of CLK protein kinase to bind and/or turn over a substrate.

The method can utilize any of the molecules disclosed in the invention. These molecules include nucleic acid molecules encoding PTP20, PCP-2, BDP1, mCLK2, mCLK3, mCLK4, or SIRP polypeptides, nucleic acid vectors, recombinant cells, polypeptides, or antibodies of the invention.

Screening Agents for Disease Treatment

In another aspect the invention features a method of screening potential agents useful for treatment of a disease or condition characterized by an abnormality in a signal transduction pathway that contains an interaction between a PTP20, PCP-2, BDP1, mCLK2, mCLK3, mCLK4, or SIRP polypeptide and a natural binding partner (NBP). The method involves assaying potential agents for those able to promote or disrupt the interaction as an indication of a useful agent.

By “NBP” is meant a natural binding partner of a PTP20, PCP-2, BDP1, mCLK2, mCLK3, mCLK4, or SIRP polypeptide that naturally associates with the polypeptide. The structure (primary, secondary, or tertiary) of the particular natural binding partner will influence the particular type of interaction between the PTP20, PCP-2, BDP1, mCLK2, mCLK3, mCLK4, or SIRP polypeptide and the natural binding partner. For example, if the natural binding partner comprises a sequence of amino acids complementary to the PTP20, PCP-2, BDP1, mCLK2, mCLK3, mCLK4, or SIRP polypeptide, covalent bonding may be a possible interaction. Similarly, other structural characteristics may allow for other corresponding interactions. The interaction is not limited to particular residues and specifically may involve phosphotyrosine, phosphoserine, or phosphothreonine residues. A broad range of sequences may be capable of interacting with the polypeptides. One example of a natural binding partner may be SHP-2. Other examples include, but are not limited to, SHP-1 and Grb2. Using techniques well known in the art, one may identify several natural binding partners for PTP20, PCP-2, BDP1, mCLK2, mCLK3, mCLK4, or SIRP polypeptides such as by utilizing a two-hybrid screen.

By “screening” is meant investigating an organism for the presence or absence of a property. The process may include measuring or detecting various properties, including the level of signal transduction and the level of interaction between a PTP20, PCP-2, BDP1, mCLK2, mCLK3, mCLK4, or SIRP polypeptide and a NBP.

By “disease or condition” is meant a state in an organism, e.g., a human, which is recognized as abnormal by members of the medical community. The disease or condition may be characterized by an abnormality in one or more signal transduction pathways in a cell wherein one of the components of the signal transduction pathway is either a PTP20, PCP-2, BDP1, mCLK2, mCLK3, mCLK4, or SIRP polypeptide or a NBP. Specific diseases or disorders which might be treated or prevented, based upon the affected cells include, but are not limited to, cancers and diabetes.

In preferred embodiments, the methods described herein involve identifying a patient in need of treatment. Those skilled in the art will recognize that various techniques may be used to identify such patients.

By “abnormality” is meant a level which is statistically different from the level observed in organisms not suffering from such a disease or condition and may be characterized as either an excess amount, intensity or duration of signal or a deficient amount, intensity or duration of signal. The abnormality in signal transduction may be realized as an abnormality in cell function; viability or differentiation state. The present invention is based in part on the determination that such abnormality in a pathway can be alleviated by action at the interaction site of SHP-2 with PTP20, PCP-2, BDP1, mCLK2, mCLK3, mCLK4, or SIRP polypeptide in the pathway. An abnormal interaction level may also either be greater or less than the normal level and may impair the normal performance or function of the organism. Thus, it is also possible to screen for agents that will be useful for treating a disease or condition, characterized by an abnormality in the signal transduction pathway, by testing compounds for their ability to affect the interaction between a PTP20, PCP-2, BDP1, mCLK2, mCLK3, mCLK4, or SIRP polypeptide and SHP-2, since the complex formed by such interaction is part of the signal transduction pathway. However, the disease or condition may be characterized by an abnormality in the signal transduction pathway even if the level of interaction between the PTP20, PCP-2, BDP1, mCLK2, mCLK3, mCLK4, or SIRP polypeptide and NBP is normal.

By “interact” is meant any physical association between polypeptides, whether covalent or non-covalent. This linkage can include many chemical mechanisms, for instance covalent binding, affinity binding, intercalation, coordinate binding and complexation. Examples of non-covalent bonds include electrostatic bonds, hydrogen bonds, and Van der Waals bonds. Furthermore, the interactions between polypeptides may either be direct or indirect. Thus, the association between two given polypeptides may be achieved with an intermediary agent, or several such agents, that connects the two proteins of interest.

Another example of an indirect interaction is the independent production, stimulation, or inhibition of both a SIRP polypeptide and SHP-2 by a regulatory agent. Depending upon the type of interaction present, various methods may be used to measure the level of interaction. For example, the strengths of covalent bonds are often measured in terms of the energy required to break a certain number of bonds (i.e., kcal/mol) Non-covalent interactions are often described as above, and also in terms of the distance between the interacting molecules. Indirect interactions may be described in a number of ways, including the number of intermediary agents involved, or the degree of control exercised over the SIRP polypeptide relative to the control exercised over SHP-2 or another NBP.

By “disrupt” is meant that the interaction between the PTP20, PCP-2, BDP1, mCLK2, mCLK3, mCLK4, or SIRP polypeptide and a NBP is reduced either by preventing expression of the polypeptide, or by preventing expression of the NBP, or by specifically preventing interaction of the naturally synthesized proteins or by interfering with the interaction of the proteins.

By “promote” is meant that the interaction between a PTP20, PCP-2, BDP1, mCLK2, mCLK3, mCLK4, or SIRP polypeptide and a NBP is increased either by increasing expression of the polypeptide, or by increasing expression of the NBP, or by decreasing the dephosphorylating activity of the corresponding regulatory PTP (or other phosphatase acting on other phosphorylated signaling components) by promoting interaction of the polypeptide and the NBP or by prolonging the duration of the interaction. Covalent binding can be promoted either by direct condensation of existing side chains or by the incorporation of external bridging molecules. Many bivalent or polyvalent linking agents are useful in coupling polypeptides, such as an antibody, to other molecules. For example, representative coupling agents can include organic compounds such as thioesters, carbodiimides, succinimide esters, diisocyanates, glutaraldehydes, diazobenzenes and hexamethylene diamines. This listing is not intended to be exhaustive of the various classes of coupling agents known in the art but, rather, is exemplary of the more common coupling agents. (See Killen and Lindstrom 1984, J. Immunol. 133:1335-2549; Jansen, F. K., et al., 1982, Immunological Rev. 62:185-216; and Vitetta et al., supra).

By “signal transduction pathway” is meant the sequence of events that involves the transmission of a message from an extracellular protein to the cytoplasm through a cell membrane. The signal ultimately will cause the cell to perform a particular function, for example, to uncontrollably proliferate and therefore cause cancer. Various mechanisms for the signal transduction pathway (Fry et al., Protein Science, 2:1785-1797, 1993) provide possible methods for measuring the amount or intensity of a given signal. Depending upon the particular disease associated with the abnormality in a signal transduction pathway, various symptoms may be detected. Those skilled in the art recognize those symptoms that are associated with the various other diseases described herein. Furthermore, since some adapter molecules recruit secondary signal transducer proteins towards the membrane, one measure of signal transduction is the concentration and localization of various proteins and complexes. In addition, conformational changes that are involved in the transmission of a signal may be observed using circular dichroism and fluorescence studies.

Diagnosis and Treatment of Disease

In another aspect the invention features a method of diagnosis of an organism for a disease or condition characterized by an abnormality in a signal transduction pathway that contains an interaction between a PTP20, PCP-2, BDP1, mCLK2, mCLK3, mCLK4, or SIRP polypeptide and a NBP. The method involves detecting the level of interaction as an indication of said disease or condition.

By “organism” is meant any living creature. The term includes mammals, and specifically humans. Preferred organisms include mice, as the ability to treat or diagnose mice is often predictive of the ability to function in other organisms such as humans.

By “diagnosis” is meant any method of identifying a symptom normally associated with a given disease or condition. Thus, an initial diagnosis may be conclusively established as correct by the use of additional confirmatory evidence such as the presence of other symptoms. Current classification of various diseases and conditions is constantly changing as more is learned about the mechanisms causing the diseases or conditions. Thus, the detection of an important symptom, such as the detection of an abnormal level of interaction between PTP20, PCP-2, BDP1, mCLK2, mCLK3, mCLK4, or SIRP polypeptides and NBPs may form the basis to define and diagnose a newly named disease or condition. For example, conventional cancers are classified according to the presence of a particular set of symptoms. However, a subset of these symptoms may both be associated with an abnormality in a particular signaling pathway, such as the ras21 pathway and in the future these diseases may be reclassified as ras21 pathway diseases regardless of the particular symptoms observed.

Yet another aspect of the invention features a method for treatment of an organism having a disease or condition characterized by an abnormality in a signal transduction pathway. The signal transduction pathway contains an interaction between a PTP20, PCP-2, BDP1, mCLK2, mCLK3, mCLK4, or SIRP polypeptide and a NBP and the method involves promoting or disrupting the interaction, including methods that target the polypeptide:NBP interaction directly, as well as methods that target other points along the pathway.

By “dominant negative mutant protein” is meant a mutant protein that interferes with the normal signal transduction pathway. The dominant negative mutant protein contains the domain of interest (e.g., a PTP20, PCP-2, BDP1, mCLK2, mCLK3, mCLK4, or SIRP polypeptide or a NBP), but has a mutation preventing proper signaling, for example by preventing binding of a second domain from the same protein. One example of a dominant negative protein is described in Millauer et al., Nature Feb. 10, 1994. The agent is preferably a peptide which blocks or promotes interaction of the PTP20, PCP-2, BDP1, mCLK2, mCLK3, mCLK4, or SIRP polypeptide and a NBP. The peptide may be recombinant, purified, or placed in a pharmaceutically acceptable carrier or diluent.

An EC50 or IC50 of less than or equal to 100 μM is preferable, and even more preferably less than or equal to 50 μM, and most preferably less that or equal to 20 μM. Such lower EC50's or IC50's are advantageous since they allow lower concentrations of molecules to be used in vivo or in vitro for therapy or diagnosis. The discovery of molecules with such low EC50's and IC50's enables the design and synthesis of additional molecules having similar potency and effectiveness. In addition, the molecule may have an EC50 or IC50 less than or equal to 100 &M at one or more, but not all cells chosen from the group consisting of parathyroid cell, bone osteoclast, juxtaglomerular kidney cell, proximal tubule kidney cell, distal tubule kidney cell, cell of the thick ascending limb of Henle's loop and/or collecting duct, central nervous system cell, keratinocyte in the epidermis, parafollicular cell in the thyroid (C-cell), intestinal cell, trophoblast in the placenta, platelet, vascular smooth muscle cell, cardiac atrial cell, gastrin-secreting cell, glucagon-secreting cell, kidney mesangial cell, mammary cell, beta cell, fat/adipose cell, immune cell and GI tract cell.

By “therapeutically effective amount” is meant an amount of a pharmaceutical composition having a therapeutically relevant effect. A therapeutically relevant effect relieves to some extent one or more symptoms of the disease or condition in the patient; or returns to normal either partially or completely one or more physiological or biochemical parameters associated with or causative of the disease or condition. Generally, a therapeutically effective amount is between about 1 nmole and 1 μmole of the molecule, depending on its EC50 or IC50 and on the age and size of the patient, and the disease associated with the patient.

The invention features a method for screening for human cells containing a PTP20, PCP-2, BDP1, mCLK2, mCLK3, mCLK4, or SIRP polypeptide or an equivalent sequence. The method involves identifying the novel polypeptide in human cells using techniques that are routine and standard in the art, such as those described herein for identifying PTP20, PCP-2, BDP1, mCLK2, mCLK3, mCLK4, or SIRP (e.g., cloning, Southern or Northern blot analysis, in situ hybridization, PCR amplification, etc.).

The invention also features methods of screening human cells for binding partners of PTP20, PCP-2, BDP1, mCLK2, mCLK3, mCLK4, or SIRP polypeptides and screening other organisms for PTP20, PCP-2, BDP1, mCLK2, mCLK3, mCLK4, or SIRP or the corresponding binding partner. The present invention also features the purified, isolated or enriched versions of the peptides identified by the methods described above.

In another aspect, the invention includes recombinant cells or tissues comprising any of the nucleic acid molecules described herein.

Diagnosis and Treatment of Abnormal Conditions

Another aspect of the invention is a method of identifying compounds useful for the diagnosis or treatment of an abnormal condition in an organism. The abnormal condition can be associated with an aberration in a signal transduction pathway characterized by an interaction between a PTP20, PCP-2, BDP1, mCLK2, mCLK3, mCLK4, or SIRP polypeptide and a natural binding partner. The method comprises the following steps: (a) adding a compound to cells; and (b) detecting whether the compound promotes or disrupts said interaction between a PTP20, PCP-2, BDP1, mCLK2, mCLK3, mCLK4, or SIRP polypeptide and a natural binding partner.

The term “abnormal condition” refers to a function in an organism's cells or tissue that deviate from a normal function in the cells or tissue of that organism. In the context of this aspect of the invention, abnormal conditions can be associated with cell proliferation or with RNA splicing.

Aberrant cell proliferative conditions include cancers such as fibrotic and mesangial disorders, abnormal angiogenesis and vasculogenesis, wound healing, psoriasis, diabetes mellitus, and inflammation.

RNA splicing is a necessary function of a cell that occurs in a cell nucleus. This process is the last step in the synthesis of messenger RNA from DNA. One molecule of RNA transcribed from DNA is tied into a lariat, incised in at least two places at the intersection of the strands, the lariat is excised, and the non-excised portion is ligated together. The modified RNA is then fit to be message RNA and is ejected from the cell nucleus to be translated into a polypeptide. Thus any aberrations that exist in an organisms ability to splice the RNA of a particular gene could result in the deficiency of a cellular agent and give rise to an abnormal condition.

Thus, regulating RNA splicing could be useful in treating cancer. For example, it is known that proteins such as Raf or src become oncogenic when made in a truncated form, such as could happen when RNA is incorrectly spliced. For this reason, the proteins of the invention might be useful for finding compounds to treat cancer. In addition, molecules involved in RNA processing have been linked to spermatogenesis. Thus, modifying RNA processing could lead to more sperm (to treat infertility) or less sperm. These methods would preferably involve CLK3 due to its high expression in the testis.

The abnormal condition can be diagnosed when the organism's cells exist within the organism or outside of the organism. Cells existing outside the organism can be maintained or grown in cell culture dishes. For cells harbored within the organism, many techniques exist in the art to administer compounds, including (but not limited to) oral, parenteral, dermal, and injection applications. For cells outside of the patient, multiple techniques exist in the art to administer the compounds, including (but not limited to) cell microinjection techniques, transformation techniques, and carrier techniques.

The term “aberration”, in conjunction with a signal transduction process, refers to a PTP20, PCP-2, BDP1, mCLK2, mCLK3, mCLK4, or SIRP polypeptide that is over- or under-expressed in an organism, mutated such that its catalytic activity is lower or higher than wild-type PTP20, PCP-2, BDP1, mCLK2, mCLK3, mCLK4, or SIRP, mutated such that it can no longer interact with a binding partner, is no longer modified by another protein kinase or protein phosphatase, or no longer interacts with a binding partner.

The term “interaction” defines the complex formed between a PTP20, PCP-2, BDP1, mCLK2, mCLK3, mCLK4, or SIRP polypeptide and a natural binding partner. Compounds can bind to either the PTP20, PCP-2, BDP1, mCLK2, mCLK3, mCLK4, or SIRP polypeptide or the natural binding partner and disrupt the interaction between the two molecules. The method can also be performed by administering a group of cells containing an aberration in a signal transduction process to an organism and monitoring the effect of administering a compound on organism function. The art contains multiple methods of introducing a group of cells to an organism as well as methods of administering a compound to an organism. The organism is preferably an animal such as a frog, mouse, rat, rabbit, monkey, or ape, and also a human.

Methods of determining a compound's effect of detecting an interaction between PTP20, PCP-2, BDP1, mCLK2, mCLK3, mCLK4, or SIRP polypeptide and natural binding partners exist in the art. These methods include, but are not limited to, determining the effect of the compound upon the catalytic activity of a PTP20, PCP-2, BDP1, mCLK2, mCLK3, mCLK4, or SIRP polypeptide, the phosphorylation state of the polypeptides or natural binding partners, the ability of the polypeptide to bind a natural binding partner, or a difference in a cell morphology.

Differences in cell morphology include growth rates, differentiation rates, cell hypertrophy, survival, or prevention of cell death. These phenomena are—simply measured by methods in the art. These methods can involve observing the number of cells or the appearance of cells under a microscope with respect to time (days).

Another aspect of the invention relates to a method of diagnosing an abnormal condition associated with cell proliferation or RNA splicing in an organism. The abnormal condition can be associated with an aberration in a signal transduction pathway characterized by an interaction between a PTP20, PCP-2, BDP1, mCLK2, mCLK3, mCLK4, or SIRP polypeptide and a natural binding partner. The method comprises the step of detecting the abnormal interaction.

The abnormal interaction can be assessed by the methods described above in reference to the identification of compounds useful for diagnosing an abnormal condition in an organism.

In another aspect, the invention features a method of administering a compound to a male organism that acts a contraceptive to reproduction. The compound can inhibit the catalytic activity of a PTP20, PCP-2, BDP1, mCLK2, mCLK3, mCLK4, or SIRP or inhibit the binding of a natural binding partner to the polypeptide.

Preferred embodiments of the methods of the invention relate to PTP20, PCP-2, BDP1, mCLK2, mCLK3, mCLK4, or SIRP polypeptides that are isolated from mammals, preferably humans, and to organisms that are mammals, preferably humans.

In another aspect, the invention provides an assay to identify agents capable of interfering with the interaction between PTP20, PCP-2, BDP1, mCLK2, mCLK3, mCLK4, or SIRP polypeptide and the polypeptide's binding partner. Such assays may be performed in vitro or in vivo and are described in detail herein or can be obtained by modifying existing assays, such as the growth assay described in Ser. No. 08/487,088, filed Jun. 7, 1995, entitled “Novel Pharmaceutical Compounds” by Tang et al. (Lyon & Lyon Docket No. 212/276) (incorporated herein by reference including any drawings) or the assays described in Ser. No. 60/005,167, filed Oct. 13, 1995, entitled “Diagnosis and Treatment of TKA-1 Related Disorders” by Seedorf et al. (Lyon & Lyon Docket No. 215/256) (incorporated herein by reference including any drawings). Another assay which could be modified to use the genes of the present invention are described in International Application No. WO 94/23039, published Oct. 13, 1994. Other possibilities include detecting kinase activity in an autophosphorylation assay or testing for kinase activity on standard substrates such as histones, myelin basic protein, gamma tubulin, or centrosomal proteins. Binding partners may be identified by putting the N-terminal portion of the protein into a two-hybrid screen or detecting phosphotyrosine of a dual specificity kinase. Fields and Song, U.S. Pat. No. 5,283,173, issued Feb. 1, 1994 and is incorporated be reference herein.

The summary of the invention described above is non-limiting and other features and advantages of the invention will be apparent from the following description of the preferred embodiments, and from the claims.

### BRIEF DESCRIPTION OF THE FIGURES

FIG. 1 shows the PTP20 nucleic acid sequence isolated from Rat-1 cells and the corresponding amino acid sequence encoded by this nucleic acid molecule.

FIG. 2 shows the nucleotide sequence and predicted amino acid sequence of PCP-2. PCP-2 nucleotide sequence (5581 bp) and deduced amino acid sequence (1430 amino acid). The predicted initiating methionine (Kozak, 1984) and putative signal peptide (von Heijne, 1986) are indicated by thin single underlining. The transmembrane domain is indicated by thick underlining. The two tandem phosphatase domains are boxed. The MAM domain is indicated by a shaded box, the Ig-like domain is shown in bold italic characters, and the four fibronectin type III-like domains are indicated by dotted underlining. The polyadenylation motif (AATAAA) is shown in bold characters.

FIG. 3 shows the nucleotide sequence of human BDP1 cDNA clone and introns. The sequence first identified by PCR cloning is bordered by arrow heads. A GC-rich track which is part of the Kozak sequence (Kozak, 1987) is indicated by a dotted line. T-rich and the AATAAA sequences required for polyadenylation are underlined.

FIG. 4 compares amino acid sequences encoded by mCLK1, mCLK2, mCLK3, and mCLK4 nucleic acid molecules cloned from mouse cells. Each amino acid sequence is encoded between a start codon and a stop codon from its respective nucleic acid molecule. Dots indicate identical amino acids and hyphens are introduced for optimal alignment. The predicted nuclear localization signals are underlined. Invariant amino acids signifying CDC2 like kinases are printed in bold. The catalytic domain is indicated by arrows. The LAMMER signature is indicated by asterisks.

FIG. 5 shows the deduced amino acid sequences of SIRP4 and SIRP1. Identical amino acids are boxed. The putative signal sequence and transmembrane region are indicated by thin and thick overlines, respectively. Three Ig-like domains are indicated by stippled overlines. Potential tyrosine phosphorylation sites are shown in bold, the C-terminal proline rich region is shaded. The location of oligonucleotides flanking the Ex region is indicated by stars.

### DESCRIPTION OF THE PREFERRED EMBODIMENTS

The present invention relates to PTP20, PCP-2, BDP1, mCLK2, mCLK3, mCLK4, or SIRP polypeptides, nucleic acids encoding such polypeptides, cells, tissues and animals containing such nucleic acids, antibodies to such polypeptides, assays utilizing such polypeptides, and methods relating to all of the foregoing.

Nucleic Acid Encoding PTP20, PCP-2, BDP1, mCLK2, mCLK3, mCLK4, or SIRP Polypeptides.

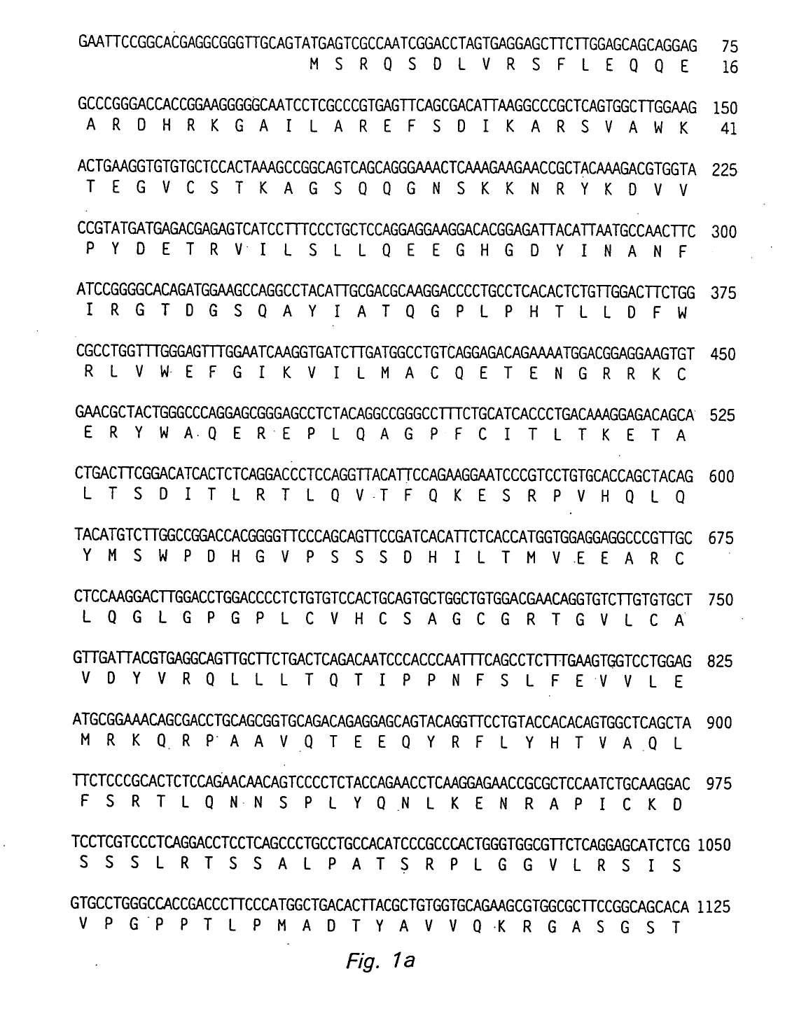

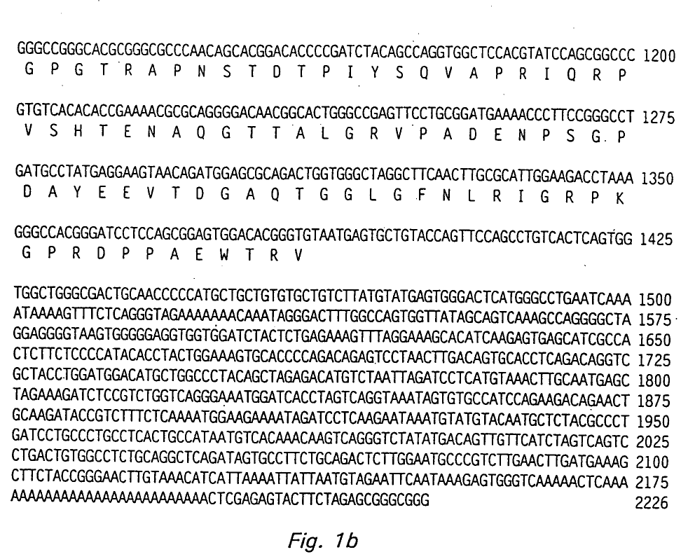

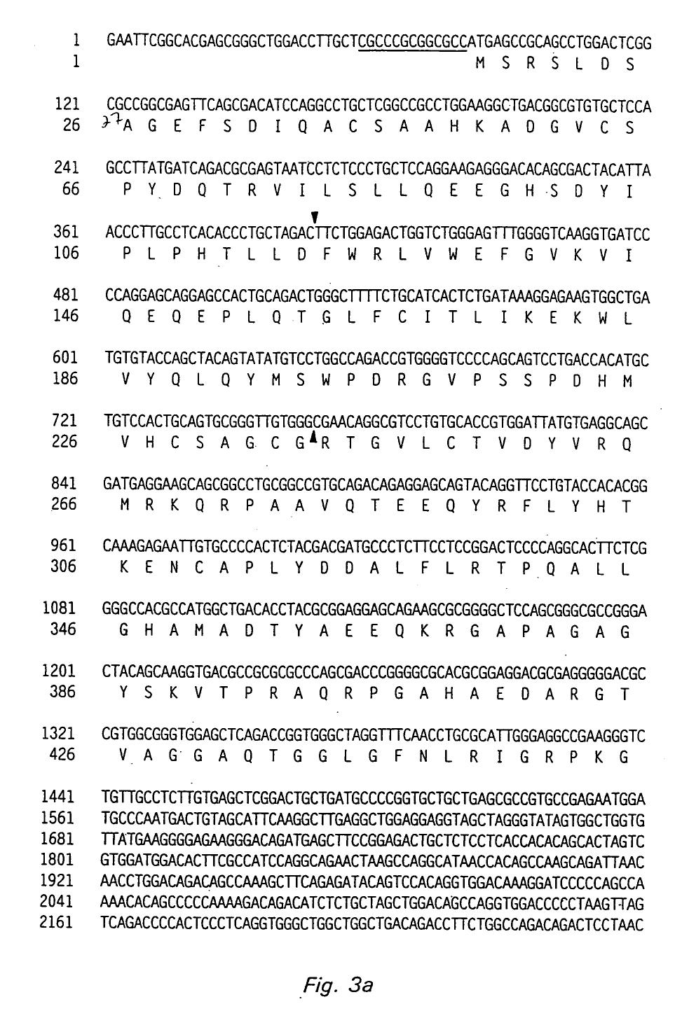

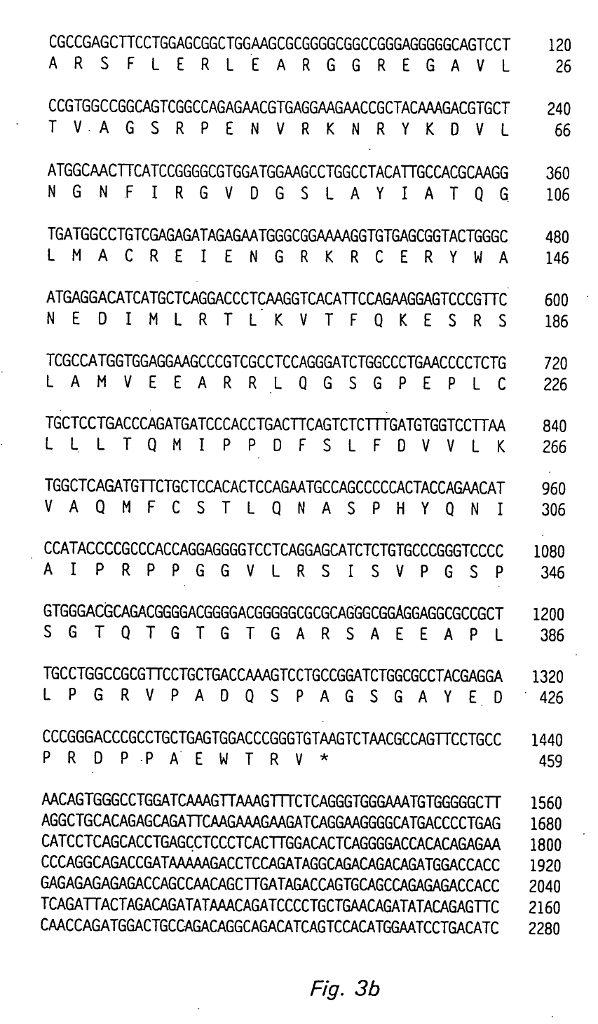

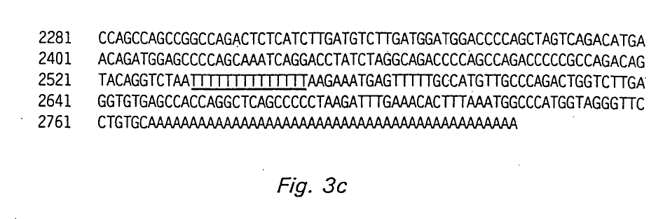

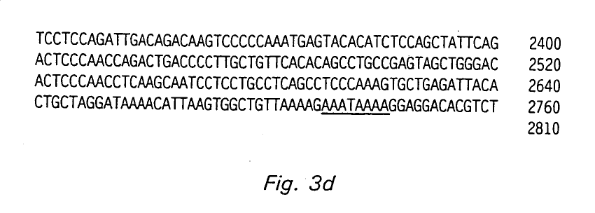

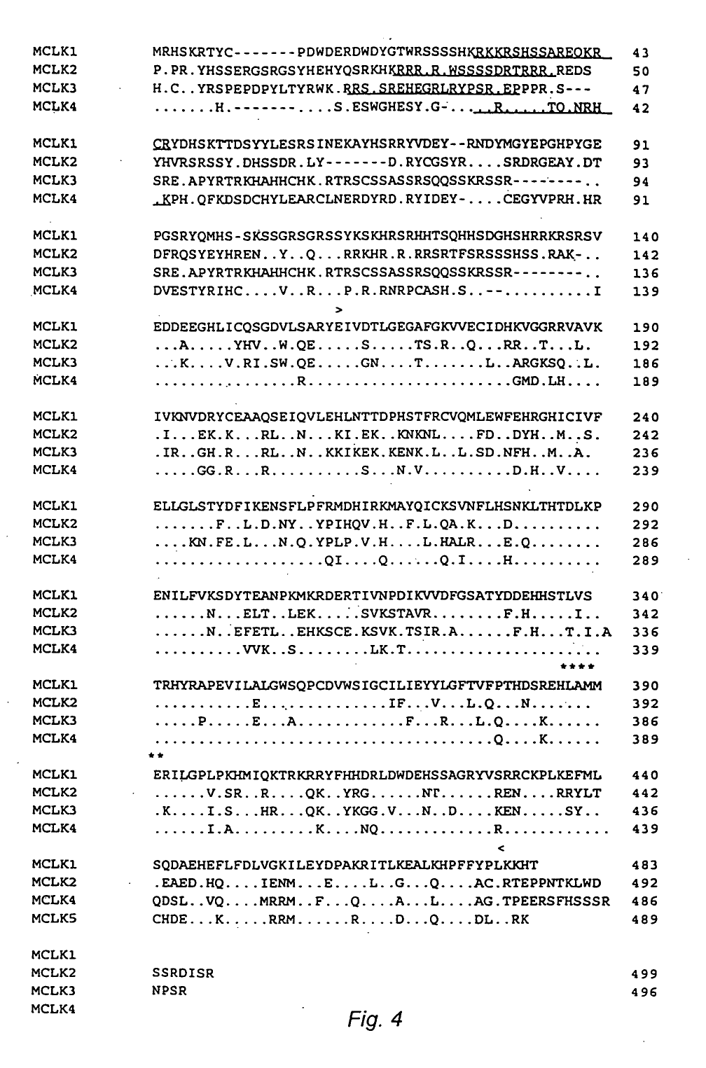

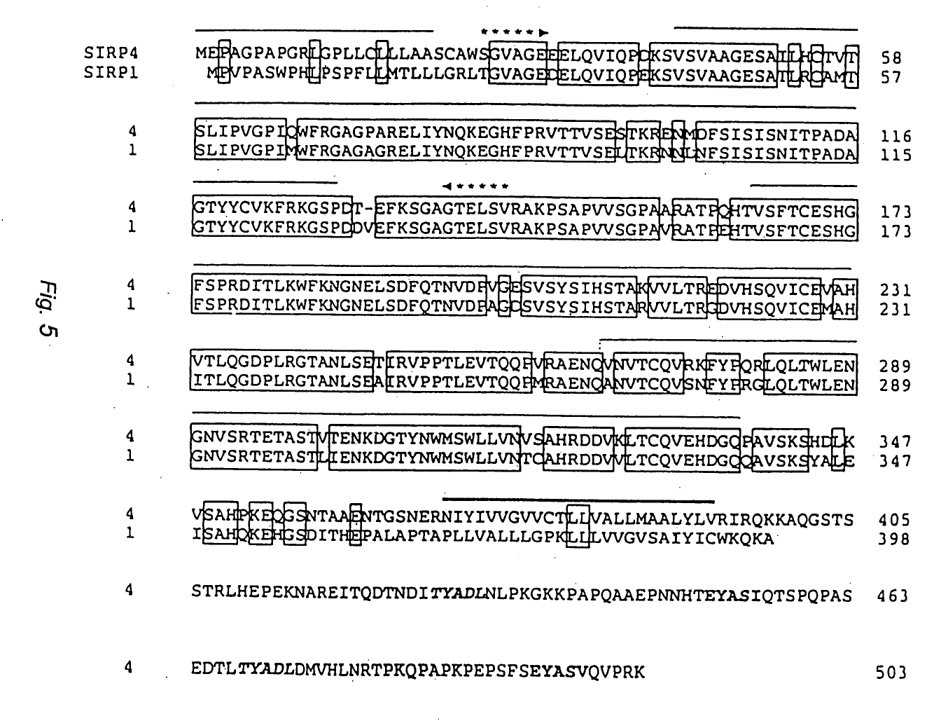

Included within the scope of this invention are the functional equivalents of the herein-described isolated nucleic acid molecules. The degeneracy of the genetic code permits substitution of certain codons by other codons which specify the same amino acid and hence would give rise to the same protein. The nucleic acid sequence can vary substantially since, with the exception of methionine and tryptophan, the known amino acids can be coded for by more than one codon. Thus, portions or all of the PTP20, PCP-2, BDP1, mCLK2, mCLK3, mCLK4, or SIRP gene could be synthesized to give a nucleic acid sequence significantly different from that shown in FIGS. 1-5. The encoded amino acid sequence thereof would, however, be preserved.

In addition, the nucleic acid sequence may comprise a nucleotide sequence which results from the addition, deletion or substitution of at least one nucleotide to the 5′-end and/or the 3′-end of the nucleic acid formula shown in FIGS. 1-5 or a derivative thereof. Any nucleotide or polynucleotide may be used in this regard, provided that its addition, deletion or substitution does not alter the amino acid sequence of FIGS. 1-5 which is encoded by the nucleotide sequence. For example, the present invention is intended to include any nucleic acid sequence resulting from the addition of ATG as an initiation codon at the 5′-end of the inventive nucleic acid sequence or its derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of the PTP20, PCP-2, BDP1, mCLK2, mCLK3, mCLK4, or SIRP genes and fragments thereof permitted by the genetic code are, therefore, included in this invention.

Further, it is possible to delete codons or to substitute one or more codons by codons other than degenerate codons to produce a structurally modified polypeptide, but one which has substantially the same utility or activity of the polypeptide produced by the unmodified nucleic acid molecule. As recognized in the art, the two polypeptides are functionally equivalent, as are the two nucleic acid molecules which give rise to their production, even though the differences between the nucleic acid molecules are not related to degeneracy of the genetic code.

A Nucleic Acid Probe for the Detection of PTP20, PCP-2, BDP1, mCLK2, mCLK3, mCLK4, or SIRP.

A nucleic acid probe of the present invention may be used to probe an appropriate chromosomal or cDNA library by usual hybridization methods to obtain another nucleic acid molecule of the present invention. A chromosomal DNA or cDNA library may be prepared from appropriate cells according to recognized methods in the art (cf. “Molecular Cloning: A Laboratory Manual”, second edition, edited by Sambrook, Fritsch, & Maniatis, Cold Spring Harbor Laboratory, 1989).

In the alternative, chemical synthesis is carried out in order to obtain nucleic acid probes having nucleotide sequences which correspond to N-terminal and C-terminal portions of the amino acid sequence of the polypeptide of interest. Thus, the synthesized nucleic acid probes may be used as primers in a polymerase chain reaction (PCR) carried out in accordance with recognized PCR techniques, essentially according to PCR Protocols, “A Guide to Methods and Applications”, edited by Michael et al., Academic Press, 1990, utilizing the appropriate chromosomal or cDNA library to obtain the fragment of the present invention.

One skilled in the art can readily design such probes based on the sequence disclosed herein using methods of computer alignment and sequence analysis known in the art (cf. “Molecular Cloning: A Laboratory Manual”, second edition, edited by Sambrook, Fritsch, & Maniatis, Cold Spring Harbor Laboratory, 1989). The hybridization probes of the present invention can be labeled by standard labeling techniques such as with a radiolabel, enzyme label, fluorescent label, biotin-avidin label, chemiluminescence, and the like. After hybridization, the probes may be visualized using known methods.

The nucleic acid probes of the present invention include RNA, as well as DNA probes, such probes being generated using techniques known in the art. The nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The sample used in the above-described methods will vary based on the assay format, the detection method and the nature of the tissues, cells or extracts to be assayed. Methods for preparing nucleic acid extracts of cells are well known in the art and can be readily adapted in order to obtain a sample which is compatible with the method utilized.

A Probe Based Method and Kit for Detecting PTP20, PCP-2, BDP1, mCLK2, mCLK3, mCLK4, or SIRP.

One method of detecting the presence of PTP20, PCP-2, BDP1, mCLK2, mCLK3, mCLK4, or SIRP in a sample comprises (a) contacting said sample with the above-described nucleic acid probe, under conditions such that hybridization occurs, and (b) detecting the presence of said probe bound to said nucleic acid molecule. One skilled in the art would select the nucleic acid probe according to techniques known in the art as described above. Samples to be tested include but should not be limited to RNA samples of human tissue.

A kit for detecting the presence of PTP2.0, PCP-2, BDP1, mCLK2, mCLK3, mCLK4, or SIRP in a sample comprises at least one container means having disposed therein the above-described nucleic acid probe. The kit may further comprise other containers comprising one or more of the following: wash reagents and reagents capable of detecting the presence of bound nucleic acid probe. Examples of detection reagents include, but are not limited to radiolabelled probes, enzymatic labeled probes (horseradish peroxidase, alkaline phosphatase), and affinity labeled probes (biotin, avidin, or streptavidin).

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris-buffers, and the like), and containers which contain the reagents used to detect the hybridized probe, bound antibody, amplified product, or the like. One skilled in the art will readily recognize that the nucleic acid probes described in the present invention can readily be incorporated into one of the established kit formats which are well known in the art.

DNA Constructs Comprising a PTP20, PCP-2, BDP1, mCLK2, mCLK3, mCLK4, or SIRP Nucleic Acid Molecule And Cells Containing these Constructs.

The present invention also relates to a recombinant DNA molecule comprising, 5′ to 3′, a promoter effective to initiate transcription in a host cell and the above-described nucleic acid molecules. In addition, the present invention relates to a recombinant DNA molecule comprising a vector and an above-described nucleic acid molecules. The present invention also relates to a nucleic acid molecule comprising a transcriptional region functional in a cell, a sequence complimentary to an RNA sequence encoding an amino acid sequence corresponding to the above-described polypeptide, and a transcriptional termination region functional in said cell. The above-described molecules may be isolated and/or purified DNA molecules.

The present invention also relates to a cell or organism that contains an above-described nucleic acid molecule and thereby is capable of expressing a peptide. The polypeptide may be purified from cells which have been altered to express the polypeptide. A cell is said to be “altered to express a desired polypeptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding an PTP20; PCP-2, BDP1, mCLK2, mCLK3, mCLK4, or SIRP gene may be obtained by the above-described methods. This region may be retained for its transcriptional termination regulatory sequences, such as termination and polyadenylation. Thus, by retaining the 3′-region naturally contiguous to the DNA sequence encoding an PTP20, PCP-2, BDP1, mCLK2, mCLK3, mCLK4, or SIRP gene, the transcriptional termination signals may be provided. Where the transcriptional termination signals are not satisfactorily functional in the expression host cell, then a 3′ region functional in the host cell may be substituted.

Two DNA sequences (such as a promoter region sequence and an PTP20, PCP-2, BDP1, mCLK2, mCLK3, mCLK4, or SIRP sequence) are said to be operably linked if the nature of the linkage between the two DNA sequences does not (1) result in the introduction of a frame-shift mutation, (2) interfere with the ability of the promoter region sequence to direct the transcription of an PTP20, PCP-2, BDP1, mCLK2, mCLK3, mCLK4, or SIRP gene sequence, or (3) interfere with the ability of an PTP20, PCP-2, BDP1, mCLK2, mCLK3, mCLK4, or SIRP gene sequence to be transcribed by the promoter region sequence. Thus, a promoter region would be operably linked to a DNA sequence if the promoter were capable of effecting transcription of that DNA sequence. Thus, to express an PTP20, PCP-2, BDP1, mCLK2, mCLK3, mCLK4, or SIRP gene, transcriptional and translational signals recognized by an appropriate host are necessary.

The present invention encompasses the expression of the PTP20, PCP-2, BDP1, mCLK2, mCLK3, mCLK4, or SIRP gene (or a functional derivative thereof) in either prokaryotic or eukaryotic cells. Prokaryotic hosts are, generally, very efficient and convenient for the production of recombinant proteins and are, therefore, one type of preferred expression system for the PTP20, PCP-2, BDP1, mCLK2, mCLK3, mCLK4, or SIRP gene. Prokaryotes most frequently are represented by various strains of E. coli. However, other microbial strains may also be used, including other bacterial strains.

In prokaryotic systems, plasmid vectors that contain replication sites and control sequences derived from a species compatible with the host may be used. Examples of suitable plasmid vectors may include pBR322, pUC118, pUC119 and the like; suitable phage or bacteriophage vectors may include γgt10, γgt11 and the like; and suitable virus vectors may include pMAM-neo, pKRC and the like. Preferably, the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the peptide will not be glycosylated. The prokaryotic host must be compatible with the replicon and control sequences in the expression plasmid.

To express PTP20, PCP-2, BDP1, mCLK2, mCLK3, mCLK4, or SIRP (or a functional derivative thereof) in a prokaryotic cell, it is necessary to operably link the PTP20, PCP-2, BDP1, mCLK2, mCLK3, mCLK4, or SIRP sequence to a functional prokaryotic promoter. Such promoters may be either constitutive or, more preferably, regulatable (i.e., inducible or derepressible). Examples of constitutive promoters include the int promoter of bacteriophage, the bla promoter of the -lactamase gene sequence of pBR322, and the CAT promoter of the chloramphenicol acetyl transferase gene sequence of pPR325, and the like. Examples of inducible prokaryotic promoters include the major right and left promoters of bacteriophage (PL and PR), the trp, recA, acZ, acI, and gal promoters of E. coli, the -amylase (Ulmanen et al., J. Bacteriol. 162:176-182 (1985)) and the (−28-specific promoters of B. subtilis (Gilman et al., Gene sequence 32:11-20 (1984)), the promoters of the bacteriophages of Bacillus (Gryczan, In: The Molecular Biology of the Bacilli, Academic Press, Inc., NY (1982)), and Streptomyces promoters (Ward et al., Mol. Gen. Genet. 203:468-478 (1986)). Prokaryotic promoters are reviewed by Glick (J. Ind. Microbiot. 1:277-282 (1987)); Cenatiempo (Biochimie 68:505-516 (1986)); and Gottesman (Ann. Rev. Genet. 18:415-442 (1984)).

Proper expression in a prokaryotic cell also requires the presence of a ribosome binding site upstream of the gene sequence-encoding sequence. Such ribosome binding sites are disclosed, for example, by Gold et al. (Ann. Rev. Microbiol. 35:365-404 (1981)). The selection of control sequences, expression vectors, transformation methods, and the like, are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Host cells which may be used in the expression systems of the present invention are not strictly limited, provided that they are suitable for use in the expression of the PTP20, PCP-2, BDP1, mCLK2, mCLK3, mCLK4, or SIRP peptide of interest. Suitable hosts may often include eukaryotic cells. Preferred eukaryotic hosts include, for example, yeast, fungi, insect cells, mammalian cells either in vivo, or in tissue culture. Mammalian cells which may be useful as hosts include HeLa cells, cells of fibroblast origin such as VERO or CHO-K1, or cells of lymphoid origin and their derivatives. Preferred mammalian host cells include SP2/0 and J558L, as well as neuroblastoma cell lines such as IMR 332 which may provide better capacities for correct post-translational processing.

In addition, plant cells are also available as hosts, and control sequences compatible with plant cells are available, such as the cauliflower mosaic virus 35S and 19S, and nopaline synthase promoter and polyadenylation signal sequences. Another preferred host is an insect cell, for example the Drosophila larvae. Using insect cells as hosts, the Drosophila alcohol dehydrogenase promoter can be used. Rubin, Science 240:1453-1459 (1988). Alternatively, baculovirus vectors can be engineered to express large amounts of PTP20, PCP-2, BDP1, mCLK2, mCLK3, mCLK4, or SIRP in insects cells (Jasny, Science 238:1653, (1987); Miller et al., In: Genetic Engineering (1986), Setlow, J. K., et al., eds., Plenum, Vol. 8, pp. 277-297).

Any of a series of yeast gene sequence expression systems can be utilized which incorporate promoter and termination elements from the actively expressed gene sequences coding for glycolytic enzymes are produced in large quantities when yeast are grown in mediums rich in glucose. Known glycolytic gene sequences can also provide very efficient transcriptional control signals. Yeast provides substantial advantages in that it can also carry out post-translational peptide modifications. A number of recombinant DNA strategies exist which utilize strong promoter sequences and high copy number of plasmids which can be utilized for production of the desired proteins in yeast. Yeast recognizes leader sequences on cloned mammalian gene sequence products and secretes peptides bearing leader sequences (i.e., pre-peptides). For a mammalian host, several possible vector systems are available for the expression of PTP20, PCP-2, BDP1, mCLK2, mCLK3, mCLK4, or SIRP.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of PTP20, PCP-2, BDP1, mCLK2, mCLK3, mCLK4, or SIRP in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288 (1982)); the TK promoter of Herpes virus (McKnight, Cell 31:355-365 (1982)); the SV40 early promoter (Benoist et al., Nature (London) 290:304-310 (1981)); the yeast gal4 gene sequence promoter (Johnston et al., Proc. Natl. Acad. Sci. (USA) 79:6971-6975 (1982); Silver et al., Proc. Natl. Acad. Sci. (USA) 81:5951-5955 (1984)).

Translation of eukaryotic mRNA is initiated at the codon which encodes the first methionine. For this reason, it is preferable to ensure that the linkage between a eukaryotic promoter and a DNA sequence which encodes PTP20, PCP-2, BDP1, mCLK2, mCLK3, mCLK4, or SIRP (or a functional derivative thereof) does not contain any intervening codons which are capable of encoding a methionine (i.e., AUG). The presence of such codons results either in a formation of a fusion protein (if the AUG codon is in the same reading frame as the PTP20, PCP-2, BDP1, mCLK2, mCLK3, mCLK4, or SIRP coding sequence) or a frame-shift mutation (if the AUG codon is not in the same reading frame as the PTP20 PCP-2, BDP1, mCLK2, mCLK3, mCLK4, or SIRP coding sequence).

A PTP20, PCP-2, BDP1, mCLK2, mCLK3, mCLK4, or SIRP nucleic acid molecule and an operably linked promoter may be introduced into a recipient prokaryotic or eukaryotic cell either as a nonreplicating DNA (or RNA) molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of single chain binding protein mRNA. These elements may include splice signals, as well as transcription promoters, enhancers, and termination signals. cDNA expression vectors incorporating such elements include those described by Okayama, Molec. Cell. Biol. 3:280 (1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColEl, pSC101, pACYC 184, “VX. Such plasmids are, for example, disclosed by Sambrook (cf. “Molecular Cloning: A Laboratory Manual”, second edition, edited by Sambrook, Fritsch, & Maniatis, Cold Spring Harbor Laboratory, (1989)). Bacillus plasmids include pC194, pC221, pT127, and the like. Such plasmids are disclosed by Gryczan (In: The Molecular Biology of the Bacilli, Academic Press, NY (1982), pp. 307-329). Suitable Streptomyces plasmids include p1J101 (Kendall et al., J. Bacteriol. 169:4177-4183 (1987)), and streptomyces bacteriophages such as .C31 (Chater et al., In: Sixth International Symposium on Actinomycetales Biology, Akademiai Kaido, Budapest, Hungary (1986), pp. 45-54). Pseudomonas plasmids are reviewed by John et al. (Rev. Infect. Dis. 8:693-704 (1986)), and Izaki (Jpn. J. Bacteriol. 33:729-742 (1978)).

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274 (1982); Broach, In: “The Molecular Biology of the Yeast Saccharomyces: Life Cycle and Inheritance”, Cold Spring Harbor Laboratory, Cold Spring Harbor, N.Y., p. 445-470 (1981); Broach, Cell 28:203-204 (1982); Bollon et al., J. Clin. Hematol. Oncol. 10:39-48 (1980); Maniatis, In: Cell Biology: A Comprehensive Treatise, Vol. 3, Gene Sequence Expression, Academic Press, NY, pp. 563-608 (1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene molecule(s) results in the production of PTP20, PCP-2, BDP1, mCLK2, mCLK3, mCLK4, or SIRP or fragments thereof. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

Purified PTP20, PCP-2, BDP1, mCLK2, mCLK3, mCLK4, or SIRP Polypeptides

A variety of methodologies known in the art can be utilized to obtain the peptide of the present invention. The peptide may be purified from tissues or cells which naturally produce the peptide. Alternatively, the above-described isolated nucleic acid fragments could be used to expressed the PTP20, PCP-2, BDP1, mCLK2, mCLK3, mCLK4, or SIRP protein in any organism. The samples of the present invention include cells, protein extracts or membrane extracts of cells, or biological fluids. The sample will vary based on the assay format, the detection method and the nature of the tissues, cells or extracts used as the sample.

Any eukaryotic organism can be used as a source for the peptide of the invention, as long as the source organism naturally contains such a peptide. As used herein, “source organism” refers to the original organism from which the amino acid sequence of the subunit is derived, regardless of the organism the subunit is expressed in and ultimately isolated from.

One skilled in the art can readily follow known methods for isolating proteins in order to obtain the peptide free of natural contaminants. These include, but are not limited to: size-exclusion chromatography, HPLC, ion-exchange chromatography, and immuno-affinity chromatography.

An Antibody Having Binding Affinity to a PTP20, PCP-2, BDP1, mCLK2, mCLK3, mCLK4, or SIRP Polypeptide and a Hybridoma Containing the Antibody.

The present invention relates to an antibody having binding affinity to a PTP20, PCP-2, BDP1, mCLK2, mCLK3, mCLK4, or SIRP polypeptide. The polypeptide may have the amino acid sequence set forth in FIGS. 1-5, or functional derivative thereof, or at least 9 contiguous amino acids thereof (preferably, at least 20, 30, 35, or 40 contiguous amino acids thereof).

The present invention also relates to an antibody having specific binding affinity to an PTP20, PCP-2, BDP1, mCLK2, mCLK3, mCLK4, or SIRP polypeptide. Such an antibody may be isolated by comparing its binding affinity to a PTP20, PCP-2, BDP1, mCLK2, mCLK3, mCLK4, or SIRP polypeptide with its binding affinity to another polypeptide. Those which bind selectively to PTP20, PCP-2, BDP1, mCLK2, mCLK3, mCLK4, or SIRP would be chosen for use in methods requiring a distinction between PTP20, PCP-2, BDP1, mCLK2, mCLK3, mCLK4, or SIRP and other polypeptides. Such methods could include, but should not be limited to, the analysis of altered PTP20, PCP-2, BDP1, mCLK2, mCLK3, mCLK4, or SIRP expression in tissue containing other polypeptides.

The PTP20, PCP-2, BDP1, mCLK2, mCLK3, mCLK4, or SIRP proteins of the present invention can be used in a variety of procedures and methods, such as for the generation of antibodies, for use in identifying pharmaceutical compositions, and for studying DNA/protein interaction.

The PTP20, PCP-2, BDP1, mCLK2, mCLK3, mCLK4, or SIRP peptide of the present invention can be used to produce antibodies or hybridomas. One skilled in the art will recognize that if an antibody is desired, such a peptide would be generated as described herein and used as an immunogen. The antibodies of the present invention include monoclonal and polyclonal antibodies, as well fragments of these antibodies, and humanized forms. Humanized forms of the antibodies of the present invention may be generated using one of the procedures known in the art such as chimerization or CDR grafting. The present invention also relates to a hybridoma which produces the above-described monoclonal antibody, or binding fragment thereof. A hybridoma is an immortalized cell line which is capable of secreting a specific monoclonal antibody.

In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands (1984); St. Groth et al., J. Immunol. Methods 35:1-21 (1980)). Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal which is immunized, the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or -galactosidase) or through the inclusion of an adjuvant during immunization.

For monoclonal antibodies, spleen cells from the immunized animals are removed, fused with myeloma cells, such as SP2/0-Agl4 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Res. 175:109-124 (1988)). Hybridomas secreting the desired antibodies are cloned and the class and subclass is determined using procedures known in the art (Campbell, “Monoclonal Antibody Technology Laboratory Techniques in Biochemistry and Molecular Biology”, supra (1984)).

For polyclonal antibodies, antibody containing antisera is isolated from the immunized animal and is screened for the presence of antibodies with the desired specificity using one of the above-described procedures. The above-described antibodies may be detectably labeled. Antibodies can be detectably labeled through the use of radioisotopes, affinity labels (such as biotin, avidin, and the like), enzymatic labels (such as horse radish peroxidase, alkaline phosphatase, and the like) fluorescent labels (such as FITC or rhodamine, and the like), paramagnetic atoms, and the like. Procedures for accomplishing such labeling are well-known in the art, for example, see (Stemberger et al., J. Histochem. Cytochem. 18:315 (1970); Bayer et al., Meth. Enzym. 62:308 (1979); Engval et al., Immunol. 109:129 (1972); Goding, J. Immunol. Meth. 13:215 (1976)). The labeled antibodies of the present invention can be used for in vitro, in vivo, and in situ assays to identify cells or tissues which express a specific peptide.

The above-described antibodies may also be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10 (1986); Jacoby et al., Meth. Enzym. 34, Academic Press, N.Y. (1974)). The immobilized antibodies of the present invention can be used for in vitro, in vivo, and in situ assays as well as in immunochromatography.

Furthermore, one skilled in the art can readily adapt currently available procedures, as well as the techniques, methods and kits disclosed above with regard to antibodies, to generate peptides capable of binding to a specific peptide sequence in order to generate rationally designed antipeptide peptides, for example see Hurby et al., “Application of Synthetic Peptides: Antisense Peptides”, In Synthetic Peptides, A User's Guide, W.H. Freeman, N.Y., pp. 289-307 (1992), and Kaspczak et al., Biochemistry 28:9230-8 (1989).

Anti-peptide peptides can be generated by replacing the basic amino acid residues found in the PTP20, PCP-2; BDP1, mCLK2, mCLK3, mCLK4, or SIRP peptide sequence with acidic residues, while maintaining hydrophobic and uncharged polar groups. For example, lysine, arginine, and/or histidine residues are replaced with aspartic acid or glutamic acid and glutamic acid residues are replaced by lysine, arginine or histidine.

An Antibody Based Method and Kit for Detecting PTP20, PCP-2, BDP1, mCLK2, mCLK3, mCLK4, or SIRP.

The present invention encompasses a method of detecting an PTP20, PCP-2, BDP1, mCLK2, mCLK3, mCLK4, or SIRP polypeptide in a sample, comprising: (a) contacting the sample with an above-described antibody, under conditions such that immunocomplexes form, and (b) detecting the presence of said antibody bound to the polypeptide. In detail, the methods comprise incubating a test sample with one or more of the antibodies of the present invention and assaying whether the antibody binds to the test sample. Altered levels of PTP20, PCP-2, BDP1, mCLK2, mCLK3, mCLK4, or SIRP in a sample as compared to normal levels may indicate disease.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluorescent assays) can readily be adapted to employ the antibodies of the present invention. Examples of such assays can be found in Chard, “An Introduction to Radioimmunoassay and Related Techniques” Elsevier Science Publishers, Amsterdam, The Netherlands (1986); Bullock et al., “Techniques in Immunocytochemistry,” Academic Press, Orlando, Fla. Vol. 1 (1982), Vol. 2 (1983), Vol. 3 (1985); Tijssen, “Practice and Theory of Enzyme Immunoassays: Laboratory Techniques in Biochemistry and Molecular Biology,” Elsevier Science Publishers, Amsterdam, The Netherlands (1985).

The immunological assay test samples of the present invention include cells, protein or membrane extracts of cells, or biological fluids such as blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is capable with the system utilized.

A kit contains all the necessary reagents to carry out the previously described methods of detection. The kit may comprise: (i) a first container means containing an above-described antibody, and (ii) second container means containing a conjugate comprising a binding partner of the antibody and a label. In another preferred embodiment, the kit further comprises one or more other containers comprising one or more of the following: wash reagents and reagents capable of detecting the presence of bound antibodies.

Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit may be as described above for nucleic acid probe kits. One skilled in the art will readily recognize that the antibodies described in the present invention can readily be incorporated into one of the established kit formats which are well known in the art.

Isolation of Compounds which Interact with PTP20, PCP-2, BDP1, mCLK2, mCLK3, mCLK4, or SIRP.

The present invention also relates to a method of detecting a compound capable of binding to a PTP20, PCP-2, BDP1, mCLK2, mCLK3, mCLK4, or SIRP polypeptide comprising incubating the compound with PTP20, PCP-2, BDP1, mCLK2, mCLK3, mCLK4, or SIRP and detecting the presence of the compound bound to PTP20, PCP-2, BDP1, mCLK2, mCLK3, mCLK4, or SIRP. The compound may be present within a complex mixture, for example, serum, body fluid, or cell extracts.

The present invention also relates to a method of detecting an agonist or antagonist of PTP20, PCP-2, BDP1, mCLK2, mCLK3, mCLK4, or SIRP activity or PTP20, PCP-2, BDP1, mCLK2, mCLK3, mCLK4, or SIRP binding partner activity comprising incubating cells that produce PTP20, PCP-2, BDP1, mCLK2, mCLK3, mCLK4, or SIRP in the presence of a compound and detecting changes in the level of PTP20, PCP-2, BDP1, mCLK2, mCLK3, mCLK4, or SIRP activity or PTP20, PCP-2, BDP1, mCLK2, mCLK3, mCLK4, or SIRP binding partner activity. The compounds thus identified would produce a change in activity indicative of the presence of the compound. The compound may be present within a complex mixture, for example, serum, body fluid, or cell extracts. Once the compound is identified it can be isolated using techniques well known in the art.

The present invention also encompasses a method of agonizing (stimulating) or antagonizing PTP20, PCP-2, BDP1, mCLK2, mCLK3, mCLK4, or SIRP associated activity in a mammal comprising administering to said mammal an agonist or antagonist to PTP20, PCP-2, BDP1, mCLK2, mCLK3, mCLK4, or SIRP in an amount sufficient to effect said agonism or antagonism. A method of treating diseases in a mammal with an agonist or antagonist of PTP20, PCP-2, BDP1, mCLK2, mCLK3, mCLK4, or SIRP related activity comprising administering the agonist or antagonist to a mammal in an amount sufficient to agonize or antagonize PTP20, PCP-2, BDP1, mCLK2, mCLK3, mCLK4, or SIRP associated functions is also encompassed in the present application.

Transgenic Animals.

A variety of methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82: 4438-4442 (1985)). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), Harlan Sprague Dawley (Indianapolis, Ind.), etc.

The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout, Experientia 47: 897-905 (1991). Other procedures for introduction of DNA into tissues of animals are described in U.S. Pat. No. 4,945,050 (Sandford et al., Jul. 30, 1990).

By way of example only, to prepare a transgenic mouse, female mice are induced to superovulate. Females are placed with males, and the mated females are sacrificed by CO2 asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice. See Hammer et al., Cell 63:1099-1112 (1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art. See, for example, Teratocarcinomas and Embryonic Stem Cells, A Practical Approach, E. J. Robertson, ed., IRL Press (1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombination. Capecchi, Science 244: 1288-1292 (1989). Methods for positive selection of the recombination event (i.e., neo resistance) and dual positive-negative selection (i.e., neo resistance and gancyclovir resistance) and the subsequent identification of the desired clones by PCR have been described by Capecchi, supra and Joyner et al., Nature 338: 153-156 (1989), the teachings of which are incorporated herein. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others. See Houdebine and Chourrout, supra; Pursel et al., Science 244:1281-1288 (1989); and Simms et al., Bio/Technology 6:179-183 (1988).

Thus, the invention provides transgenic, nonhuman mammals containing a transgene encoding a PTP20, PCP-2, BDP1, mCLK2, mCLK3, mCLK4, or SIRP polypeptide or a gene effecting the expression of a PTP20, PCP-2, BDP1, mCLK2, mCLK3, mCLK4, or SIRP polypeptide. Such transgenic nonhuman mammals are particularly useful as an in vivo test system for studying the effects of introducing a PTP20, PCP-2, BDP1, mCLK2, mCLK3, mCLK4, or SIRP polypeptide, regulating the expression of a PTP20, PCP-2, BDP1, mCLK2, mCLK3, mCLK4, or SIRP polypeptide (i.e., through the introduction of additional genes, antisense nucleic acids, or ribozymes).

A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. The transgenic DNA may encode for a human PTP20, PCP-2, BDP1, mCLK2, mCLK3, mCLK4, or SIRP polypeptide. Native expression in an animal may be reduced by providing an amount of anti-sense RNA or DNA effective to reduce expression of the receptor.

Gene Therapy

PTP20, PCP-2, BDP1, mCLK2, mCLK3, mCLK4, or SIRP or its genetic sequences will also be useful in gene therapy (reviewed in Miller, Nature 357:455-460, (1992). Miller states that advances have resulted in practical approaches to human gene therapy that have demonstrated positive initial results. The basic science of gene therapy is described in Mulligan, Science 260:926-931, (1993).

In one preferred embodiment, an expression vector containing the PTP20, PCP-2, BDP1, mCLK2, mCLK3, mCLK4, or SIRP coding sequence is inserted into cells, the cells are grown in vitro and then infused in large numbers into patients. In another preferred embodiment, a DNA segment containing a promoter of choice (for example a strong promoter) is transferred into cells containing an endogenous PTP20, PCP-2, BDP1, mCLK2, mCLK3, mCLK4, or SIRP in such a manner that the promoter segment enhances expression of the endogenous PTP20, PCP-2, BDP1, mCLK2, mCLK3, mCLK4, or SIRP gene (for example, the promoter segment is transferred to the cell such that it becomes directly linked to the endogenous PTP20, PCP-2, BDP1, mCLK2, mCLK3, mCLK4, or SIRP gene).

The gene therapy may involve the use of an adenovirus containing PTP20, PCP-2, BDP1, mCLK2, mCLK3, mCLK4, or SIRP cDNA targeted to a tumor, systemic PTP20, PCP-2, BDP1, mCLK2, mCLK3, mCLK4, or SIRP increase by implantation of engineered cells, injection with PTP20, PCP-2, BDP1, mCLK2, mCLK3, mCLK4, or SIRP virus, or injection of naked PTP20, PCP-2, BDP1, mCLK2, mCLK3, mCLK4, or SIRP DNA into appropriate tissues.

Target cell populations may be modified by introducing altered forms of one or more components of the protein complexes in order to modulate the activity of such complexes. For example, by reducing or inhibiting a complex component activity within target cells, an abnormal signal transduction event(s) leading to a condition may be decreased, inhibited, or reversed. Deletion or missense mutants of a component, that retain the ability to interact with other components of the protein complexes but cannot function in signal transduction may be used to inhibit an abnormal, deleterious signal transduction event.

Expression vectors derived from viruses such as retroviruses, vaccinia virus, adenovirus, adeno-associated virus, herpes viruses, several RNA viruses, or bovine papilloma virus, may be used for delivery of nucleotide sequences (e.g., cDNA) encoding recombinant PTP20, PCP-2, BDP1, mCLK2, mCLK3, mCLK4, or SIRP protein into the targeted cell population (e.g., tumor cells). Methods which are well known to those skilled in the art can be used to construct recombinant viral vectors containing coding sequences. See, for example, the techniques described in Maniatis et al., Molecular Cloning: A Laboratory Manual, Cold Spring Harbor Laboratory, N.Y. (1989), and in Ausubel et al., Current Protocols in Molecular Biology, Greene Publishing Associates and Wiley Interscience, N.Y. (1989). Alternatively, recombinant nucleic acid molecules encoding protein sequences can be used as naked DNA or in reconstituted system e.g., liposomes or other lipid systems for delivery to target cells (See e.g., Felgner et al., Nature 337:387-8, 1989). Several other methods for the direct transfer of plasmid DNA into cells exist for use in human gene therapy and involve targeting the DNA to receptors on cells by complexing the plasmid DNA to proteins. See, Miller, supra.

In its simplest form, gene transfer can be performed by simply injecting minute amounts of DNA into the nucleus of a cell, through a process of microinjection. Capecchi M R, Cell 22:479-88 (1980). Once recombinant genes are introduced into a cell, they can be recognized by the cells normal mechanisms for transcription and translation, and a gene product will be expressed. Other methods have also been attempted for introducing DNA into larger numbers of cells. These methods include: transfection, wherein DNA is precipitated with CaPO4 and taken into cells by pinocytosis (Chen C. and Okayama H, Mol. Cell. Biol. 7:2745-52 (1987)); electroporation, wherein cells are exposed to large voltage pulses to introduce holes into the membrane (Chu G. et al., Nucleic Acids Res., 15:1311-26 (1987)); lipofection/liposome fusion, wherein DNA is packaged into lipophilic vesicles which fuse with a target cell (Felgner P L., et al., Proc. Natl. Acad. Sci. USA. 84:7413-7 (1987)); and particle bombardment using DNA bound to small projectiles (Yang N S. et al., Proc. Natl. Acad. Sci. 87:9568-72 (1990)). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene. Curiel D T et al., Am. J. Respir. Cell. Mol. Biol., 6:247-52 (1992).

As used herein “gene transfer” means the process of introducing a foreign nucleic acid molecule into a cell. Gene transfer is commonly performed to enable the expression of a particular product encoded by the gene. The product may include a protein, polypeptide, anti-sense DNA or RNA, or enzymatically active RNA. Gene transfer can be performed in cultured cells or by direct administration into animals. Generally gene transfer involves the process of nucleic acid contact with a target cell by non-specific or receptor mediated interactions, uptake of nucleic acid into the cell through the membrane or by endocytosis, and release of nucleic acid into the cytoplasm from the plasma membrane or endosome. Expression may require, in addition, movement of the nucleic acid into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the patient.

In another preferred embodiment, a vector having nucleic acid sequences encoding a PTP20, PCP-2, BDP1, mCLK2, mCLK3, mCLK4, or SIRP is provided in which the nucleic acid sequence is expressed only in specific-tissue. Methods of achieving tissue-specific gene expression as set forth in International Publication No. WO 93/09236, filed Nov. 3, 1992 and published May 13, 1993.

In all of the preceding vectors set forth above, a further aspect of the invention is that the nucleic acid sequence contained in the vector may include additions, deletions or modifications to some or all of the sequence of the nucleic acid, as defined above.

In another preferred embodiment, a method of gene replacement is set forth. “Gene replacement” as used herein means supplying a nucleic acid sequence which is capable of being expressed in vivo in an animal and thereby providing or augmenting the function of an endogenous gene which is missing or defective in the animal.

All of these aspects and features are explained in detail with respect to the protein PYK-2 in PCT publication WO 96/18738, which is incorporated herein by reference in its entirety, including any drawings. Those skilled in the art will readily appreciate that such descriptions can be easily adapted to PTP20, PCP-2, BDP1, mCLK2, mCLK3, mCLK4, or SIRP as well, and is equally applicable to the present invention.

### EXAMPLES

The examples below are non-limiting and are merely representative of various aspects and features of the present invention. The examples below demonstrate the isolation and characterization of the novel proteins PTP20, PCP-2, BDP1, mCLK2, mCLK3, mCLK4, or SIRP proteins. The experiments identify the full length nucleic and amino acid sequences for the proteins and study the expression interaction and signalling activities of such proteins. The nucleotide sequence for human BDP1 has been deposited in the GenBank data base under accession number X79568.

### Example 1

### Identification and Cloning of New Proteins

The same general methods were used to identify and clone the new PTPs and PTKs of the invention. Briefly, degenerate oligonucleotide primers based on consensus sequences in known PTPs and PTKs were used to generate PCR fragments using RNA isolated from specific cell types. Total RNA was isolated by the guanidinium thiocyanate/CsCl procedure (Ullrich, et al., Science 196:1313, 1977; Chirgwin, et al., Biochemistry 18:5294, 1979). Poly(A)+ RNA was isolated using oligo (dT)-cellulose chromatography. The PCR fragments were isolated, subcloned into pBluescript cloning vectors (Stratagene), and sequenced using the dideoxynucleotide chain termination method (Sanger, et al., PNAS 74:5463, 1977). Fragments representing previously unknown proteins were used as hybridization probes to identify full-length clones in cDNA libraries. The specific procedures used for each of the proteins of the invention are described in detail below.

PTP20—

The degenerate primers used to identify PTP20 were FWXMXW (sense) and HCSAG(S/I/V)G (antisense). Random-primed cDNA (up to 50 ng) from PC12 cell RNA was used as a template. Both sense and antisense primers were added to a 100 ml reaction mixture containing 20 mM Tris-HCl (pH8.4), 50 mM KCl, 2.5 mM MgCl2, 0.01% BSA, all four dNTPs (each at 200 mM), 1 unit of Taq polymerase (Boehringer Mannheim) and template cDNA. Thirty-five cycles were carried out on a thermal cycler; each cycle involved incubation at 94° C. for 1 min, at 42° C. for 1 min and 72° C. for 1 min. The PCR products were separated on a 1.5% agarose gel. Fragments of 350-400 bp were excised, subcloned and sequenced.

The novel PTP20 PCR fragment was isolated, radioactively labeled by random priming, and used to screen 1×106 plaques from a PC12 cDNA library which had been made using a pool of poly(A)+RNA from both undifferentiated and differentiated PC12 cells, and a ZAPII synthesis kit (Stratagene). Hybridization was performed in a solution containing 50% (v/v) formamide, 5×SSC, 5×Denhardt solution, 0.05M sodium phosphate, 1 mM NaH2PO4, 1 mM Na4P207, 0.1 mM ATP, 5 mg salmon sperm DNA at 42° C. for 20 h. Washing was repeated three times with 2×SSC/0.1% SDS for 20 min at 42° C. Positive clones were plaque-purified by secondary screening, rescued according to the manufacturer's instruction and sequenced in both directions. The 2226 bp cDNA clone of PTP20 contained an open reading frame of 1359 bp, encoding a protein of 453 amino acids with a predicted MW of 50 kDa, preceded by 27 base pairs of 5′-non-coding region and 840 base pairs of 3′-non-coding region. The 3′-non-coding region contained the polyadenylation signal sequence AATAAA.

BDP1—

We used sequence homology and PCR amplification to clone the protein tyrosine phosphatases expressed in human brain tissue. The degenerate primers for PCR were designed according to the consensus sequences from alignment of amino acid sequences of known PTPases. The longest consensus sequences FWXMXW and HCSAGXG in catalytic domains were selected. A single-lane sequencing of 379 amplified cDNA clones identified 15 different cDNA clones, including CD45, LAR, MEG1, PTPase, PTPase, PTPase, PTPase, PTPase, PTPase and PTPase 1D. One clone encoded a novel putative protein tyrosine phosphatase. We called the clone BDP1 because it was found in human brain cDNA.

The CR-amplified BDP1 clone was used for screening cDNA libraries. Screened first were the cDNA libraries related to human brain tissue, such as fetal brain, amygdala and pituitary. Comparison of the nucleotide sequence of the BDP1 PCR product and 1.1 Kb BDP1 from human fetal brain cDNA library revealed introns in the fetal brain clone. More than half of 23 positive clones were found to be imperfectly spliced. As is already known, these intron sequences start as GT and end as AG. We tried specific PCR primers, designed on the basis of sequence comparison, to differentiate between complete clones and incomplete ones with intron sequences. Three introns of 367, 80 and 91 bp-long sequences were found at the position of nucleotide 733, 799 and 878, respectively (FIG. 1B). The locations of introns are indicated by arrow heads in FIG. 1A.

Thirty-six different cDNA libraries were examined with a pair of specific primers. PCR of cDNA clones with and without intron sequence would produce 725 bp and 358 bp bands, respectively. Six amplified PCR reactions, which showed bands around the 358 bp position, were taken and Southern blot hybridization was performed with 32p-labelled BDP1 PCR clone. Only one cDNA library, constructed from MED01 hematopoietic cell line, showed the positive Southern signal (data not shown). Eight positive clones were obtained from the MEGO1 cDNA library and confirmed to have a poly(A)+tail.

The degenerate primers used to identify BDP1 were FWXMXW (sense) and HCSAG(S/I/V)G (antisense). 2 μg of human brain poly(A)+RNA were used for the synthesis of the first-strand cDNA, employing oligo(dT)-priming and RNase H-negative reverse transcriptase (GIBCO/BRL). 50 ng of synthesized cDNA were amplified with 30 pmol of each degenerate primer in 100 μl of PCR solution for 30 cycles. Amplified PCR products were digested with BamHI or EcoRI and separated on 6% acrylamide gel. Fragments of about 350 bp were excised, subcloned and sequenced.

The 360 bp PCR product, named BDP1, was identified to be a novel PTPase clone. Specific sense and antisense primers were synthesized according to the comparison of the nucleotide sequence of the BDP1 PCR product and 1.1 Kb BDP1 from human fetal brain cDNA library. 2 μl of cDNA library solutions were used for PCR with specific primers. 20 μl of amplified solutions were analyzed on 1.6% agarose gel electrophoresis and blotted onto a nitrocellulose filter for Southern hybridization. The BDP1 PCR product was 32P-labelled with random priming (USB) and used as a probe for Southern blotting and screening of cDNA libraries. Positive clones from MEGOI cDNA library in Zap II were picked up and rescued for sequencing. Nucleotides of the longest 2.8 Kb cDNA clone were sequenced in both directions.

The longest clone from the MEGO1 cDNA library was 2810 bp long and contained a single long open reading frame (ORF) of 1377 bp which was preceded by a 5′-noncoding region without a stop codon. Its overall G+C content was 57%. There were no long ORF in the 3′-noncoding sequence. This clone had no intron sequences that were detected in other clones. Only both 5′- and 3′-flanking primer regions were slightly different, but the 340 bp sequence between primers perfectly matched the BDP1 PCR product (see box in FIG. 1A).

The ATG at the beginning of the ORF was flanked by a sequence that conforms to the Kozak consensus for translation initiation like the GC-rich track (Kozak, M. (1987). Nucleic Acids Res. 15, 8125-8248). Purine base was identified in position −3 and A instead of G in position +4. The 3′-noncoding region contains two distinct sequence elements which are required for accurate and efficient polyadenylation (15). One element T-rich sequence was located 200 nucleotides downstream and another AAATAAAA was 20 nucleotides downstream from the poly(A)+tail; The two elements are underlined in FIG. 1A.

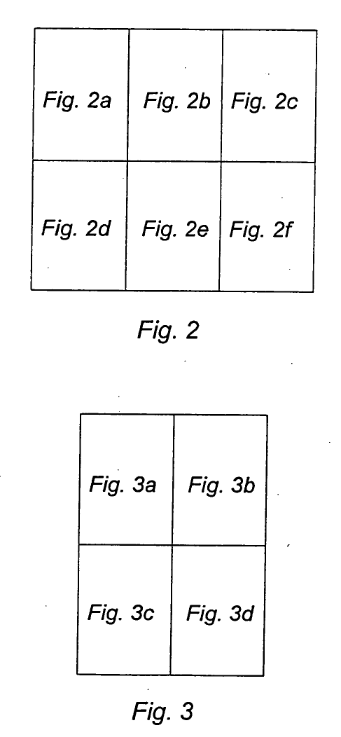

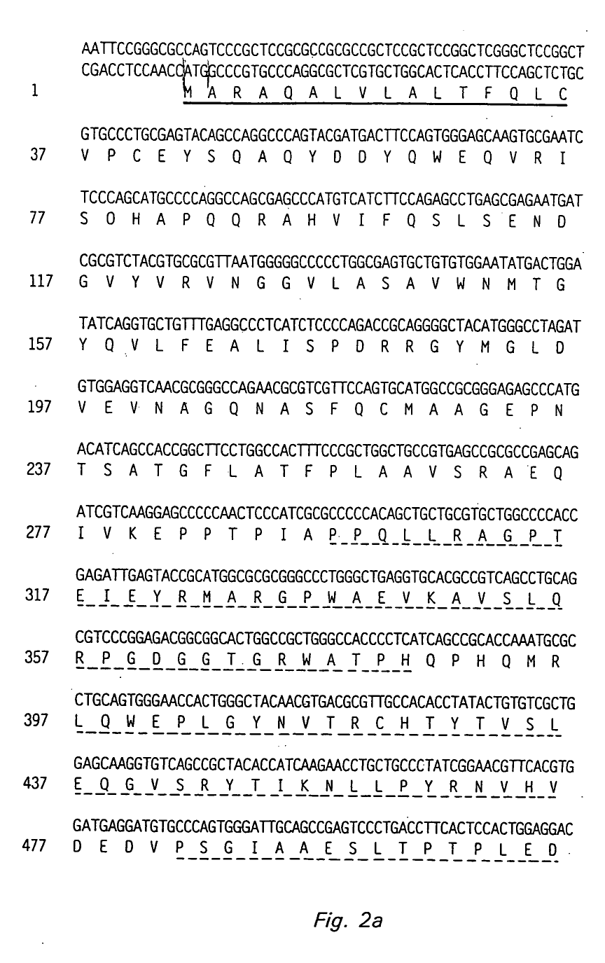

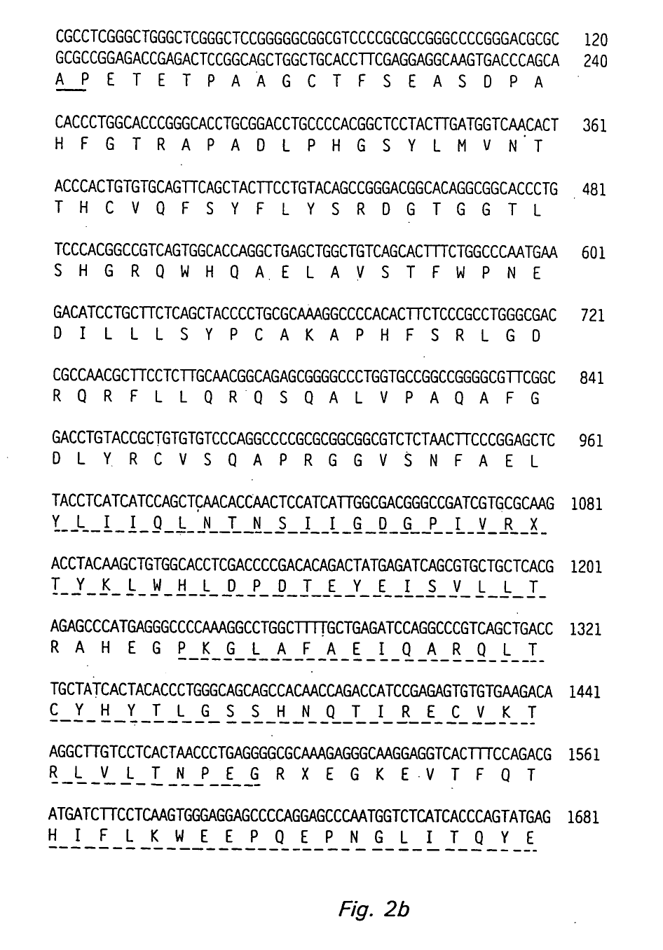

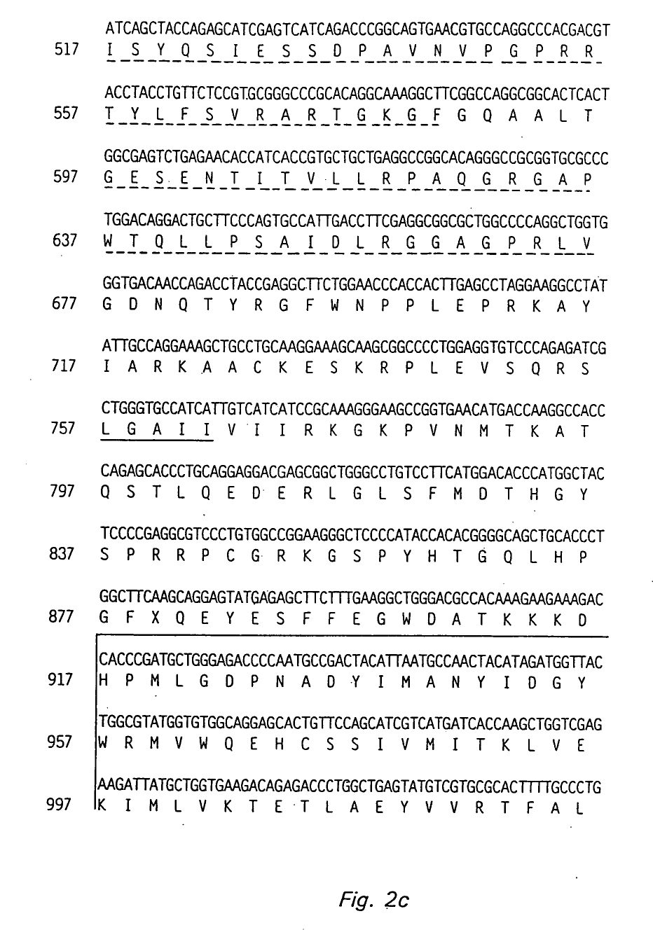

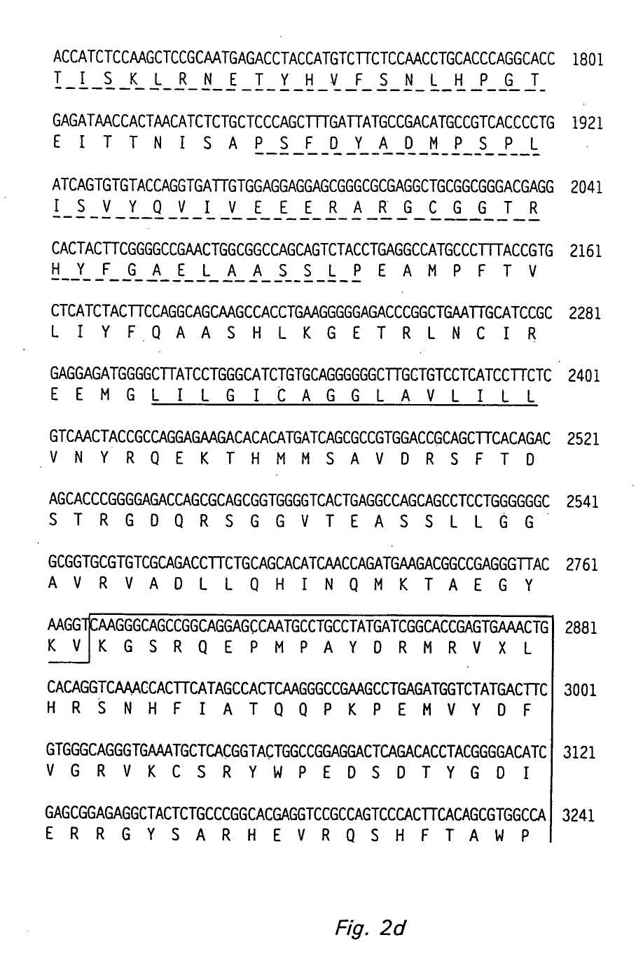

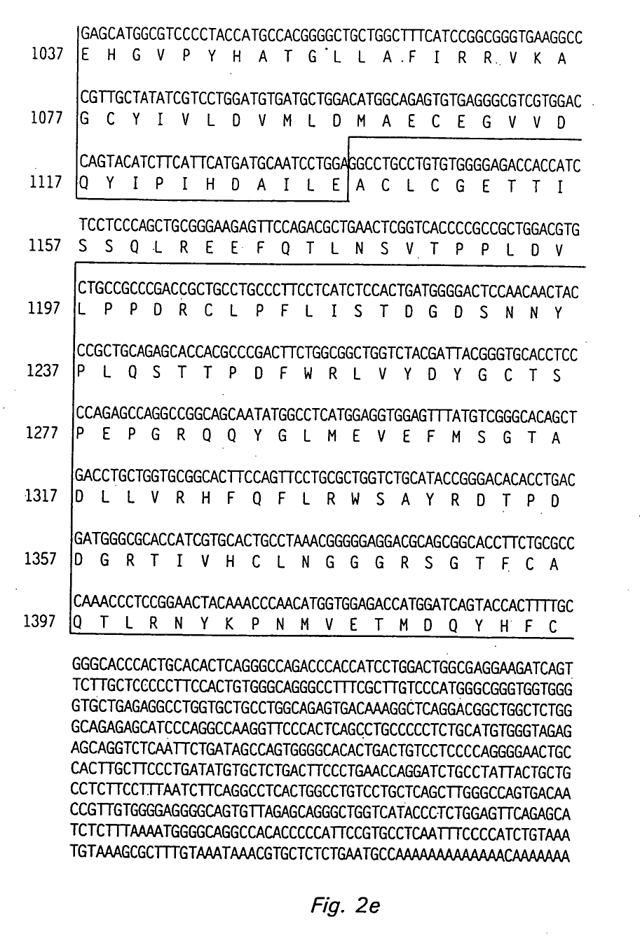

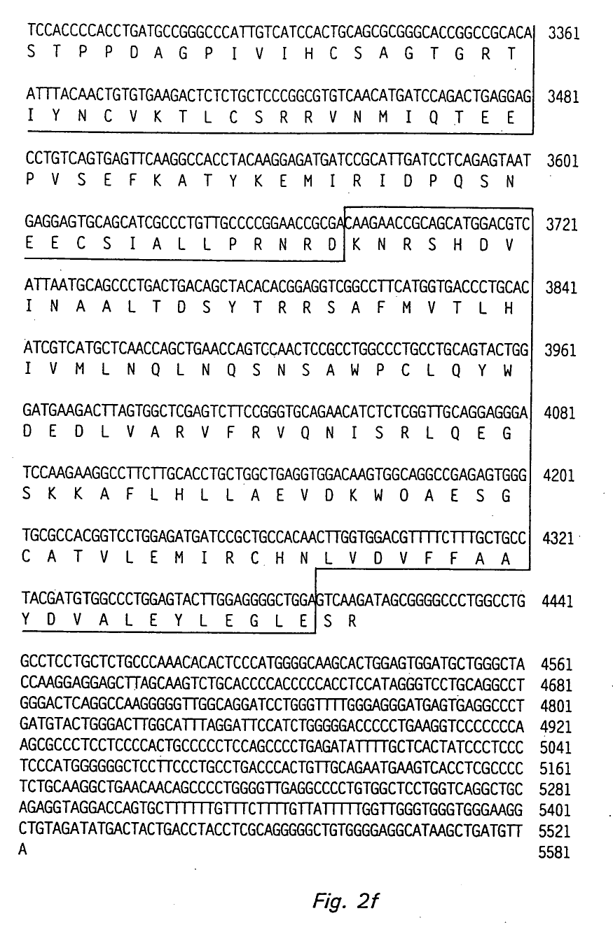

The ORF of BDP1 is a residue with 459 amino acids, and it encodes a protein of approximately 50 KDa. The putative catalytic region of predicted protein sequence—amino acids 59 to 294—contains all of the highly conserved sequence motifs found in most protein tyrosine phosphatases, including a Cys and Arg in the phosphate-binding loop, with these being essential for PTPase catalytic activity (Barford, D., Flint, A. J. and Tonks, N. K. (1994) Science 263, 1397-1404; Stuckey, et al. (1994). Nature 370, 571-575; Su, et al. (1994) Nature 370, 575-578; Zhang, et al. (1994) Proc. Natl. Acad. Sci. USA 91, 1624-1627). The highly conserved amino acid residues are shown in the boxes in FIG. 2A.

The mutant BDP1, whose Cys changed to Ser by site-directed mutagenesis, had no phosphatase activity on pNPP. This result suggests that the Cys residue at the active site is very important for the BDP1 activity just like for other PTPases. This region of BDP1 sequence exhibited 36% to 38% homology with the PTP-PEST-family phosphatases, such as human and rat PTPase-PESTs (Takekawa, et al. (1992) Biochem. Biophys. Res. Comm. 189, 1223-1230; Yang, et al. (1993) J. Biol. Chem. 268, 6622-6628) and PEP PTPase (Matthews, et al. (1992). Mol. Cell. Biol. 12, 2396-2405). Other known PTPases exhibited less than 34% homology.

The deduced amino acid sequence from aa 1 to 25 at the N-terminus was compared with sequences in data banks. It was found that the 70 KDa cyclase-associated CAP protein of yeast (Field, et al. (1990) Cell 61, 319-327), rat (Selicof, et al. (1993) J. Biol. Chem. 268, 13448-13453) and human (Matviw, et al. (1992) Mol. Cell. Biol. 12, 5033-5040) were homologous, as is illustrated in FIG. 2B. Especially the FLERLE sequence could also be found in the acidic FGF molecule near the second Cys consensus residue, and was also reported to take part in the binding to its own receptor molecule on the cell surface (Thomas, et al. (1991). Ann. New York. Acad. Sci. 9-17).

Nowadays, several kinds of domains such as SH2, SH3 and PK on proteins are known to play an essential role in protein-protein interaction in signal transduction so as to overcome their low intracellular concentrations. The N-terminal part of CAP was linked to yeast Ras-signaling which was associated with the adenylate cyclase protein (25). CAP protein is known to be essential for yeast growth, but its role in higher eukaryote cells is still unknown. The CAP-homologous domain of BDP1 may be expected to play a role in protein-protein association.

The 160 aa-long-tail sequence from the 295th amino acid residue has no homology with known proteins, nor do PEST motifs (Rogers, et al. (1986). Science 234, 364-368). The PTPase-PEST family has a long tail containing the nuclear localization signal in PEP (Flores, et al. E., Roy, G., Patel, D., Shaw, A. and Thomas, M. L. (1994) Mol. Cell. Biol. 14, 4938-4946) and the serine phosphorylation site in human PTPase-PEST (Farton, A. J. and Tonks, N. K. (1994) PTP-PEST: a protein tyrosine phosphatase regulated by serine phosphorylation. EMBO J. 13, 3763-3771). All these sequences are not contained in BDP1 PTPase. The amino acid composition of P, E, S and T of BDP1 at the tail sequence were 11.4, 4.8, 6.0 and 6.6%, respectively. The E, S and T contents were much lower, but P was higher than the PTPase-PEST-family phosphatases. The molecular weight of BDP1, namely 50 KDa, was much lower than that of PTPase-PEST (88 KDa) and that of hematopoietic PTPase-PEST (90 KDa). The short half-life of PTPase in cells, due to the PEST motif, must still be investigated. However, the BDP1 sequence of the last 22 amino acids at the carboxy terminus were similar to two PTPases with PEST motif, as shown in FIG. 2C.

Besides the cytoplasmic tail sequences of transmembrane proteins, MHC-IA and HLA-DQ were homologous with the BDP1 C-terminus (Malissen, et al. (1983). Science 221, 750-754; Kappes, et al. (1988) Ann. Rev. Biochem. 57, 991-1028). The last C-terminal sequence contains many Pro residues, so it seems to be a Pro-rich sequence for binding to the SH3 domain. It also contains a Trp residue which is difficult to replace during the evolution period. This suggests that its C-terminal portion might be essential for protein function, such as cellular localization or even regulation of its own activity. The hydrophobicity of this part of the molecule is not as high as PTPase 1B and T-cell PTPase, which has the function of binding to the membrane as well as controlling its own PTPase activity (Brown-Shimer, S., Johnson, K. A., Lawrence, J. B., Johnson, C., Bruskin, A., Green, N. R. and Hill, D. E. (1990) Proc. Natl. Acad. Sci. USA 87, 5148-5152; Cool, et al. (1989) Proc. Natl. Acad. Sci. USA 86, 5257-5261).

PTPases can be generally grouped into the receptor type and cytosolic type. To confirm its type, the hydrophobicity profile of BDP1 was drawn using a computer program with window size 7 (Kyte and Doolittle, J. Mol. Biol. 157, 105, 1982). It was confirmed that BDP1 has no transmembrane part and that it belongs to the group of intracellular PTPases. The average hydrophobicity of BDP1 was much higher than that of other PEST-family PTPases.

PCP-2—

PCR reactions were performed using degenerate oligonucleotide primers corresponding to the consensus sequences RWXMXW and HCSAG (S/I/V) G, and the GeneAmp® kit (Perkin-Elmer/Cetus) and pool of poly(A)+RNA from 9 human pancreatic carcinoma cell lines: A590, A818-7, AsPc 1, BxPC-2, Capan-1, Capan-2, Colo357, DAN-G and SW850 (ATCC, Rockville, Md.). The PCR fragments were isolated, subcloned, and sequenced.

A PCR fragment coding for 114 amino acids of the catalytic domain of PCP-2 was used as a probe in the screening of human pancreatic adenocarcinoma and human breast carcinoma cDNA libraries using standard filter hybridization techniques. Fifty positive clones were identified, isolated, excised in vivo, and analyzed. Two of these clones, H44 (4.6 Kb), containing a poly(A)+tail, and H13 (3.8 Kb), containing the N-terminal start codon, were sequenced with T3 and T7 primers or with synthetic oligonucleotide primers based on existing sequence data. Comparison of the PCP-2 sequence with various sequence databases were carried out using the GCG sequence analysis software package (Genetics Computer Group, Madison Wis.). The composite full-length nucleotide sequence of PCP-2 contains a consensus initiation codon (Kozak, Nucleic Acids Res. 12:857, 1984) at position 133 and is followed by a hydrophobic region that may serve as a signal peptide (von Heijne, Nucleic Acids Res. 14:4683, 1986). The translation initiation codon is followed by a single open reading frame of 4290 bp encoding 1430 amino acids, and a 3′ untranslated region of 1122 bp, including a consensus polyadenylation signal (AATAAA) upstream from the poly(A) tail of clone H44. A single transmembrane-spanning alpha-helical segment is predicted at amino acid positions 741-764. This feature delineates a putative extracellular region of 740 residues and an intracellular portion of 666 residues. The “intracellular” region contains two tandemly-repeated domains with significant similarity to the catalytic domains of previously described PTPs (Brady-Kalnay, et al., Ade. Protein Phosphatases 8:241, 1994).

The extracellular region of PCP-2 shows 53% homology to mouse PTPkappa and 47% to human or mouse PTPμ, and less than 24% similarity to other R-PTPs, such as MPTP delta, type D (Mizuno, et al., FEBS 355:223, 1994). The first approximate 160 amino acids of PCP-2 show similarity (21%) to a region in the Xenopus cell surface protein A5 and to the MAM domain of PTPkappa and PTPμ. The MAM domain of PCP-2 is followed by one Ig-like and four putative fibronectin type III-like repeats (residues 287 to 570), which are homologous to similar domains in PTPμ, PTPkappa and LAR, structural motifs that have also been previously identified in several other cell-surface molecules, such as the cell-adhesion molecule N-CAM (Cunningham, et al., Science 236:799, 1987; Mauro, et al., J. Cell Biol. 119:191, 1992).

Unique features that distinguish PCP-2 include the greater distance between its transmembrane segment and the start of the first phosphatase homology domain, a region that is rich in serine and threonine residues and exceeds that of other R-PTPs by about 60 residues, a characteristic shared by its closest relatives PTP-kappa and PTPμ. Moreover, PCP-2 contains the tripeptide HAV at position 331 to 333 of the extracellular domain, which is implicated in cell-cell contact in members of the cadherin family (Blaschuk, et al., J. Mol. Biol. 211:679, 1990). In addition, there are 13 potential N-linked glycosylation sites found in the PCP-2 extracellular domain.

### Example 2

### Expression Analysis of PTPs

The expression of the various proteins of the invention was evaluation using a standard Northern blot procedure. Poly(A)+RNA was isolated with oligo(dT) Sepharose (Stratagene) column chromatography according to the manufacturer's instruction then electrophoresed in a formaldehyde/1.0% agarose gel (2-3 mg/lane), blotted to a nitrocellulose membrane filter through capillary action overnight. The blotted filter was heated at 80° C. under vacuum for 2 hours. The filter was probed with a 32P-labeled nucleic acid probe specific for the protein under evaluation. After hybridization in a solution containing 50% (v/v) formamide for 24 hours at 42° C., the blot was washed under high stringency conditions 2×SSC, twice for 15 min at room temperature, then 0.1×SSC twice at 42° C. for 30 min, and then exposed to X-ray film at −70° C. with intensifying screen.

PTP20—

To elucidate the role of PTP20 in the differentiation process of PC12 cells, Northern blot analysis was used to examine the expression pattern of PTP20 mRNA in PC12 cells treated with NGF for three or six days. Full-length PTP20 was used as the probe. Untreated PC12 cells exhibited a 2.3 kb PTP20 mRNA transcript. Following 3 days of NGF treatment, a 1.5-fold increase in the amount of transcript was observed. Another 3 days of NGF treatment caused a 2.4-fold increase as compared to untreated cells. In addition to the predominant 2.3 kb transcript, a faint band with 1.5 kb in size was also detected which also increased in abundance as NGF treatment continued. The expression pattern of PTP20 mRNA suggested that PTP20 might play a role during NGF-induced PC12 differentiation.

BDP-1—

Expression was evaluated in both normal human tissues and tumor cell lines obtainable at the ATCC (normal: brain, fetal liver, pancreas, stomach, kidney, spleen, liver colon, placenta, heart, Calu6, MEG01, TF-1, K562, Caki-1, Sw620, RF-1, KatoIII, MDA-MB-231, Mel Gerlach, Neurofibroma). The probe was a 2 Kb EcoR1/BamH1 fragment of the full-length BDP-1. There was no expression detected in normal tissues. Expression was high in epithelial cell lines such as Caki-1 (kidney), SW620 (colon), MDA-MB-231 (breast), Calu6 (lung) and Mel Gerlach (melanoma). Basal expression was detected in MEG01 and TF-1 (hematopoietic), K-562 (CML) and RF-1 and KatoIII (gastric). This expression pattern suggests a role for BDP-1 in certain cancers.

PCP-2—

One of the PCR fragments (H44, see Example 1) was used to probe a blot of various human tissues. PCP-2 was highly expressed in brain and skeletal muscle and somewhat in pancreas. There was minor expresion in uterus and none in colon, kidney, liver, placenta, spleen and stomach.

### Example 3

### Expression of Recombinant PTPs

PTP20—

The insert of PTP20 was excised with EcoRI digestion and integrated into an expression vector, pcDNA3 (Invitrogen) which had been digested with the same restriction enzyme. The direction of the insert in the plasmid was confirmed by restriction mapping. Rat-1 cells were transfected with the plasmid (2 mg/1×106 cells) by using Lipofectin (GIBCO BRL). After 48 h of culturing, the cells were washed with PBS and then lysed with lysis buffer [50 mM HEPES, pH 7.5, containing 150 mM NaCl, 1 mM EDTA, 10% (v/v) glycerol, 1% (v/v) Triton X-100, 1 mM phenylmethylsulfonyl fluoride, 1 mM sodium orthovanadate, 10 mg/ml aprotinin]. Protein concentrations of cell lysates were measured with a protein assay kit (Bio-Rad) using bovine serum albumin as a standard. Equivalent amounts of protein were used for Western blot analyses and phosphatase activity assay.

The PTP20 mutant containing a cysteine to serine alteration at position 229 was generated using a oligonucleotide primer, CTCTGTGTCCACAGCAGTGCTGGCTGT. Kunkel, PNAS 82:488, 1985.) The mutation was confirmed by DNA sequencing.

For Western blot analysis, cells were first lysed in lysis buffer. To assess PTP20 expression, equivalent amounts of protein in the cell lysates were separated by 10% SDS-PAGE and electrophoretically transferred to nitrocellulose membranes. The membranes were first incubated with rabbit anti-PTP-PEST antibodies, and then a peroxidase-coupled goat anti-rabbit secondary antibody (BioRad) was added, followed by an enhanced chemiluminescence (ECL) substrate (Amersham) reaction. The substrate reaction was detected on a X-ray film (Amersham). The anti-PTP-PEST antibody was raised against the C-terminal 56 amino acids of human PTP-PEST (Takekawa et al., 1992, Biochem. Biophys. Res. Commun. 189:1223-1230) which was expressed as a GST fusion protein.

BDP-1—

For expression of BDP1 in an eukaryotic cell, we constructed a BDP1 cDNA expression vector based on the cytomegarovirus promoter (pRK5RS) as for PCP-2 (see below). 2 μg of BDP1 expression vector were transfected into human kidney embryonic 293 cell (ATCC CRL 1573) by the slightly modified method of Chen and Okayama (Mol Cell Bio 7:2745, 1987). 293 cells were maintained in DMEM with 10% fetal calf serum (FCS) at 5% CO2. 4×105 cells/3.5-cm dish were grown for 1.5 days. The cells were moved for transfection to 3% C02 and cultured for 17 hours after addition of DNA to the cell medium. Media were replaced with fresh normal DMEM containing 10% FCS and cultured overnight.

Recombinant expression of BDP-1 was evaluated by immunoprecipitation using an anti-PTP Pest antibody and by Western blot. the C-terminus of PTPase BDP1 is homologous with the same part of PTPase-PEST. To prepare the cell lysates, cultured cells were solubilized in 50 mM Hepes, pH 7.5, 150 mM NaCl, 1.5 mM MgCl22, 1 mM EGTA, 1% Triton X-100, 10 Mm PMSF and 1 μg/ml aprotinin, and their clear supernatant was collected after microcentrifugation at 13,000 rpm. The immunoprecipitation involved incubation of the 35S-Met-labelled cell lysates with the anti-C-terminal portion of PTPase-PEST fusion protein of GST antibody for one hour. Protein A-sepharose was added and mixed by tumbling for one hour. Protein A-sepharose beads were recovered and washed three times with 1 ml of 20 mM Hepes buffer, pH 7.5, containing 150 mM NaCl, 0.1% Triton X-100, 10% glycerol, 0.2 mM sodium orthovanadate and 10 mM sodium pyrophosphate. The washed beads were dissolved in SDS-sample buffer, the released proteins were subjected to 10% SDS-PAGE, and autoradiography was performed.

For Western blot hybridization, 10 μl of cell lysates with and without transfection of BDP1 were electrophoresized on SDS-polyacrylamide gel, blotted onto a nitrocellulose filter, hybridized with antibody and displayed with ECL (Amersham). Anti-src antibody and anti-C-terminal antibody of PTPase-PEST were used in the same solution for hybridization in order to see the src and BDP1 band from the same blot. Both experiments showed BDP1 PTPase of 50 KDa on 10% SDS-PAGE.

PCP-2—

Two cDNA clones which contained N-terminal (clone H13) and C-terminal (clone H44) fragments were used to assemble a full-length PCP-2 cDNA. Clone H44 was digested with BamHI and HindIII and cloned into pRK5RS, a cytomegalovirus (CMV) promoter-based expression vector with a modified polylinker, yielding plasmid 16/RS. The N-terminal portion of Clone H13 was then cloned into the corresponding SacI sites of 16/RS in the appropriate orientation, yielding construct PCP-2/F1, containing the full-length PCP-2 cDNA, but without the pPML CMV region of pRK5RS. PCP-2 cDNA was then released from PCP-2/F1 and recloned between XbaI and Hind III sites into pRK5RS expression vector. Human embryonic kidney fibroblast 293 cells (ATCC CRL 1573) were transfected with CsCl-purified plasmid DNA PCP-2/pRK5RS using the method described in the art (Eaton, et al., Biochemistry 25:8345, 1986; Lammers, et al. J. Biol. Chem. 268:22456, 1993).

Western blot analysis was done to confirm recombinant expression of PCP-2. 12-15 hours after transfection, cells were washed in phosphate-buffered saline and lysed in Triton X-100 lysis buffer (50 mM HEPES; pH 7.5, 150 mM NaCl, 1.5 mM MgCl2, 1 mM EGTA, 10% glycerol, 1% Triton X-100, 200 μg of phenylmethylsulfonyl fluoride per ml, 100 mM NaF, 10 μg of aprotinin per ml, 10 μg of leupeptin per ml, and 1 mM sodium orthovanadate) at 4° C. Cell lysates from PCP-2 transfected cells and control plasmid-transfected cells were separated on a 7% polyacrylamide gel, transferred to nitrocellulose, and probed with anti-PCP-2/H44-5 antibody (see below). A protein of apparent Mr 180 kDa was recognized in transfected cells which exceeded the calculated size of 160 kDa. This band was not detected in cells transfected with an empty expression vector. Detection of the 180 kDa band was blocked by preincubation with the GST-fusion protein/H44-5 (see below).

To determine whether the protein product obtained in transfected 293 cells contained N-linked carbohydrates, we treated samples with endo-F before SDS-polyacrylamide gel electrophoresis and immunoblotting. Cell cultures transfected with PCP-2 cDNA and control plasmid were harvested in lysis buffer containing 1% sodium dodecyl sulfate (SDS) by heating at 100% C for 5 min. The total lysate was vortexed and then incubated at 37% C overnight in the presence of 0.25 U of endoglycosidase F/N-glycosidase F (Boehringer Mannheim), 40 mM potassium phosphate (pH 7.0), 20 mM EDTA, 1% N-octylglucoside, 0.1% SDS and 1% β-mercaptoethanol. The total lysate was directly loaded on a 7% SDS-polyacrylamide gel and blotted with antiserum PCP-2/H44-5 Following glycosidase treatment, the mobility of the 180 kDa protein was reduced to 160 kDa, a size that matched the calculated molecular weight.

### Example 4

### Production of Specific Antibodies

PCP-2-specific immunoreagents were generated by immunizing rabbits with the bacterially expressed C-terminal 169 amino acids (residues 1070 to 1239) amino acid portion of PCP-2 expressed as a GST-fusion protein by subcloning it tnot the fusion expression vector PGEX 2T (Pharmacia). Fusion protein was purified as described (Smith, et al., Gene, 67:31-40, 1988). Polyclonal anti-serum was generated by repeatedly immunizing rabbits at two week intervals. Affinity-purified antibody was obtained by binding serum IgG to PCP-2-GST-fusion protein immobilized on glutathione-sepharose and eluting with low pH and high salt.

### Example 5

### Assays for PTP Activity

Phosphatase activity was measured for each of the PTPs of the invention using a synthetic substrate, p-nitrophenylphosphate (pNPP). In brief, purified protein was incubated in a solution containing 25 mM MES (2-[N-morpholino]ethanesulfonic acid), pH 5.5, 1.6 mM DTT, 10 mM p-nitrophenylphosphate as a substrate and 50 mg protein of cell lysate at 37° C. for 30 min. (In the case of PCP-2, 25 mM HEPES [pH 7.2] was used in place of MES.) The reaction was stopped by the addition of 100 ml of 1N NaOH, and the absorbance was measured at 405 nm.

PTP20—

Rat-1 fibroblast cells were transiently transfected with mammalian expression constructs encoding either PTP20 or a Cys to Ser mutant of PTP20. (See Example 3) Cell lysates were prepared and protein concentrations were determined. The expression level of both wild type and catalytically inactive mutant PTP20 was confirmed by Western blotting with anti-PTP-PEST antibodies. Cross-reactivity with non-specific proteins was not detected as evidenced by lack of a signal in control reactions (wt Rat-1 cells). Nearly equivalent amounts of expressed protein were detected. The size of the detected protein was 50 kDa which is consistent with the predicted molecular weight of PTP20. For protein tyrosine phosphatase activity, equivalent amounts of protein from the transfected Rat-1 cell lysates were tested using p-NPP as a substrate. Lysates from transfected cells exhibited a approximately 2.5-fold higher PTP activity over those from control cells, whereas only basal levels of PTPase activity were detected in lysates from cells transfected with a construct encoding a catalytically inactive mutant of PTP20. These results indicate that full length PTP20 cDNA encodes a functionally active PTP.

BDP-1—

The PTPase activity of recombinant BDP-1 isolated transfected 293 cells against pNPP was tested as described above. The BDP1 phosphoesterase activity of pNPP was higher at acidic pH than alkaline pH just as is the case for other PTPases.

In order to elucidate the function of BDP1, we investigated the dephosphorylating activity of BDP1 on several receptor-mediated autophosphorylations by contransfection with chimeric Tks into 293 cells (src, EGF (HER), PDGF (EP), insulin (EIR) and Kit (EK)). Chimeric receptor molecules with extracellular EGF receptors were used, since such are experimentally and quantitatively practical and enable activation of all receptor autophosphorylations to be evoked by the same concentration of EGF (100 ng/ml). After separating the proteins on 8% SDS-PAGE and blotting onto nitrocellulose filter, the upper portion of the filter containing chimeric receptor molecules and the lower portion containing BDP1 protein were hybridized with anti-phosphotyrosine antibody and polyclonal antibody against PTPase-PEST, respectively, to confirm the BDP1 expression. BDP1 acted on HER-, EP- and EK-autophosphorylation actively and on EIR partially.

BDP1 PTPase showed dephosphorylating activity on the tyrosine residue of src itself and other intracellular proteins. Transfection of only src into cells causes a high rate of tyrosine-phosphorylation in many proteins including src. Upon cotransfection of src and BDP1, the expressed BDP1 could dephosphorylate src and other proteins as well. BDP1 could not remove all the phosphoryl groups on the tyrosine residues of src protein. Although the expressed level of BDP1 increased, the remaining phosphorylating level on src did not change. This means some autophosphorylated tyrosine residue(s) on src protein are resistant to the action of BDP-1.

Even though PTPase BDP1 was overexpressed in 293 cells, some phosphoryl groups on receptors could resist the action to BDP1. The result suggests that BDP1 PTPase may play a housekeeping role to maintain itself and may have enzymatic specificity to intracellular substrate as well.

PCP-2—

PCP-2 was isolated from transiently transfected 293 cells using wheat germ agglutinin (WGA, Sigma) and its activity determined against pNPP as described above. PCP-2-transfected 293 cells deiplayed 2.5-fold higher pNPP phophastase activity than control plasmid-transfected cells. Both the PTP activities of control and PCP-2-transfected cells were reduced after pervanadate (a known PTP inhibitor) treatment.

### Example 6

### Biological Activity of PTP20

To further elucidate the function of PTP20 in cellular differentiation, PC12 cells were stably transfected with the PTP20 cDNA mammalian expression construct (infra). The transfected cells were cultured in Dulbecco's modified Eagle's medium (DMEM) containing high glucose (4.5 g/liter) supplemented with 10% heat-inactivated horse serum (HS) and fetal calf serum (FCS). 5×10⁵ cells per 60 mm dish were incubated overnight in 4 ml of growth medium. The following day, the dish was washed once with serum-free medium and then incubated with a Lipofectin (5 ml)-DNA (2 mg) mixture for 6 h. After 48 h, selection started in growth medium containing 500 mg/ml G418 (GIBCO BRL). Following 5 weeks of selection, discrete colonies were subcloned and expanded.

In parental PC12 cells, endogenous PTP20 protein was beneath detection with the antibody. Three independent clones showing high levels of PTP20 expression by Western blot appeared morphologically similar to parental PC12 cells. However, following NGF treatment (50 ng/ml), all three clones showed accelerated neurite outgrowth, with 20 to 40% of the cells expressing neurites of more than two cell bodies in length at day 1 and more than 70% of the cells expressing such neurites at day 3. In contrast, the parental PC12 cells showed less than 5% of the cells with neurites of two cell bodies in length at day 1 and 47% at day 3. At day-4 following NGF treatment, more than 70% of both parental PC12 cells and PTP-PC12, cells expressed neurite outgrowth, however, the neurite length and the abundance of neurites in PTP-PC12 cells appeared longer and larger than those of parental PC12 cells. In addition, PTP-PC12 cells responded to lower concentrations of NGF then did parental PC12 cells. This suggests that NGF-induced differentiation was promoted by the expression of PTP20 nad that PTP20 may play an important role in the growth and survival of neurons.

### Example 7

### Biological Activity of PCP-2

Immunofluorescence studies were used to examine the potential biological role of PCP-2 in regulating cell:cell interaction. SW850 human pancreatic adenocarcinoma cells (ATCC) were grown to approximately 50% confluency and fixed with 2% paraformaldehyde in phosphate buffered saline. Unspecific antibody binding was blocked with phosphate-buffered gelatin (PBG). Incubation with primary antibodies was done at room temperature for 2 h after dilution in PBG, 1:100 for purified polyclonal anti-PCP-2-antibody, 1:200 for monoclonal anti-β-catenin, and 1:400 for monoclonal anti-E-cadherin antibody (Transduction Laboratories, Lexington, Ky.). Primary antibody binding was detected with isotype specific secondary antibody, FITC (DTAF)-conjugated donkey-anti-rabbit IgG (1:200), or Cy3-conjugated goat-anti-mouse IgG (1:300, Jackson Laboratories, West Grove, Pa.). For double labeling experiments, antibody decoration was done consecutively. Controls were incubated with either anti-PCP-2/H44-5 antibody mixed with a fiftyfold excess of antigen (GST-fusion protein), or with species-specific non-immune serum, or without primary antibody under otherwise identical conditions. Coverslips were viewed with appropriate filter blocks for fluorescein and rhodamine on a LSM 410 laser scanning microscope (Carl Zeiss, Oberkochen, FRG) using a 40× oil immersion objective of aperture 1.3. To simultaneously visualize the localization of antibody binding with the cellular morphology, a gray scale transmission image (pseudo-phase contrast) and the two individual laser confocal images were superimposed in AVS (Advanced Visual Systems, Waltham, Mass.).

After seeding, SW850 cells rapidly formed a semiconfluent monolayer with prominent cell-cell contacts between neighboring cells in focal clusters. Anti-PCP-2 antibody binding was detected mostly along these intracellular adhesions. In double labeling experiments with either anti β-catenin or anti E-cadherin antibody, colocalization of the cell adhesion proteins with anti-PCP-2 was observed at cell-cell contacts. Only background label was detectable in the cytosol or Golgi area of these cells as well as in controls after antigen/antibody incubation, after no-immune serum incubation, or after incubation with primary antibody.

### Example 8

### Identification and Cloning of CLKs

The signature sequences HRDLAAR in the catalytic subdomain VI and D(V/M)WS(Y/F)G in subdomain IX were used to create degenerate oligonucleotides. (Ciossek et al., Oncogene 11:2085, 1995.) Reverse transcriptase PCR reactions were performed with 2 μg of total RNA prepared from confluent or differentiated (day 7) mouse C2C12 myoblasts (Lechner et al., PNAS 93:4355, 1996). (Ciossek et al., Oncogene 11:2085, 1995.) Briefly, 2 μg of RNA were reverse transcribed in the presence of 1 μM degenerate antisense primer, 250 μM of each nucleotide and 75 units of Stratascript reverse transcriptase (Stratagene) in a total volume of 20 μl for 30 min at 42° C. 2 μl of the above reaction was used in a PCR reaction using degenerate sense and antisense oligonucleotides (1 μM each), 25 μM of each nucleotide and 2.5 units Taq polymerase (Boehringer). 30 cycles were performed with 1 min for each 94° C., 50° C. and 72° C. step. Fragments of approximately 250 bp were gel purified, cloned in Bluescript and sequenced.

mCLK2, mCLK3 and mCLK4 were cloned from a mouse embryo 11.5 p.c. 1ZAP cDNA library (Ciossek et al., supra) using the isolated PCR fragment as a probe according to manufacturer's instructions (final wash in 0.5×SSC/0.1% SDS at 42° C.) (Stratagene) mCLK1 was cloned by reverse transcriptase PCR from 1 μg brain poly(A)+ RNA using specific primers mCLKls-Bam, CGGGATCCCTTCGCCTTGCAGCTTTGTC and mCLKlas-EcoRI, CGGAATTCCTAGACTGATACAGTCTGTAAG, and Pwo polymerase (Doehringer).

From the approximately 300 fragments which were sequenced from the first PCR reaction, one was novel. It resembled a member of the LAMMER family of dual specificity kinases (Yun et al., Genes. Dev. 8:1160, 1994), also known as CLK kinases (Ben-David et al., EMBO J. 10:317, 1991) or STY (Howell et al., Mol. Cell. Biol. 11:568, 1991) and shared a high homology to a part of the human cDNA hCLK2. Full length clones of this and three related proteins were obtained from a mouse embryonic cDNa library as described. The same libraries were rescreened with a mixture of mCLK1, 2, 3, and 4 fragments at low stringency to isolate additional novel members of this family. Reverse transcriptase PCR reactions were performed on brain, kidney and liver poly(A)+ RNA with degenerate primers coding for the DLKPEN and AMMERI motifs. These efforts did not identify additional genes.

### Example 9

### Expression Analysis of CLKs

RNA was extracted from frozen adult mice tissues or tissue culture cells including normal liver, testis, lung, brain, kidney and thyroid and F9, P19 (embryonic carcinomas), TT-HD (ovary teratoma), F-MEL (Friend murine erythroleukemia), NF 561 (myeloid leukemia) and WEHI-3B (myelomonocyte) cell lines. (Puissant and Houdebine, Biotechniques 8:148, 1990.) 10 μg total RNA was then electrophoresed in 1.2% agarose formaldehyde gels (Sambrook et al., 1989, Cold Spring Harbour Laboratory Press) and transferred to Hybond N membranes (Amersham). Hybridization was performed overnight in 50% formamide, 5×SSC (750 mM sodium chloride, 75 mM sodium citrate), 5×Denhardt's (0.1% Ficoll 400, 0.1% polyvinylpyrrolidone, 0.1% BSA), 0.2% SDS and 100 μg/ml salmon sperm DNA. 1-3×10⁶ cpM/ml of 32p-random primed DNA probe (Amersham Megaprime kit) was used, followed by washes at 0.2×SSC/0.1% SDS at 42° C. Blots were incubated with Hyperfilm-MP (Amersham) at −80° C. for 2 weeks. Membranes were stripped for reuse by boiling in 0.1% SDS/water.

Differences in expression patterns were observed for the CLK genes, especially in testes. Low mCLK1 expression levels were observed in testes as compared to mCLK2, mCLK3 and mCLK4. However, while almost all of the mCLK3 message represented the catalytically active splice form, mCLK4 was expressed predominantly as a message encoding the truncated protein. mCLK2 was also highly expressed in this tissue, but as a larger transcript. Similar large transcripts, which did not correspond to the expected message size, were detected for all mCLK genes which most likely represented non- or partially spliced messages in analogy to mCLK1. (Duncan et al., J. Biol. Chem. 270:21524, 1995.) The ratio of these larger RNA species, when compared to the coding mRNA, varied among the CLK kinases.

Because it was reported (Ben-David et al., EMBO J. 10:317, 1991) that mCLK1 kinase was over-expressed in certain cancer cell lines, studies were extended to mCLK1-4. Although messages for the four genes were detected in all cell lines tested, albeit in sometimes very low quantities, significant differences of expression levels between the cell lines for each individual gene were observed. However, an overall increase of mCLK mRNA was not detected in transformed cells, even though higher levels of particular mCLK messages were detected in some cell. Low expression levels were detected in WEHI and NF561 cell lines, with the majority of the messages representing the splice form encoding the truncated product. The mRNA expression levels of mCLK1-4 genes were investigated in the C2C12 cell line and Li adipocytes during differentiation, but no noticeable change in expression was detected.

### Example 10

### Expression of Recombinant CLKs

GST fusion constructs were generated by subcloning full length mCLK1, mCLK2, mCLK3 and mCLK4 cDNAs by PCR into pGEX vectors (Pharmacia), creating in-frame glutathione S-transferase (GST) fusion constructs using the-following primers for PCR: mCLKls-Bam (as above); mCLKlas-Not I, TATAGCGGCCGCTAGACTGATACAGTCTGT; mCLK2s-Sma I, TCCCCCGGGATGCCCCATCCCCGAAGGTACCA; mCLK2 as-Not I, TATAGCGGCCGCTCACCGACTGATATCCCGACTGGAGTC; mCLK3s-Sma I, TCCCCCGGGGAGACGATGCATCACTGTAAG; mCLK3 as-Not I, TATAGCGGCCGCGCTGGCCTGCACCTGTCATCTGCTGGG; mCLK4s-EcoRI, CGGAATTCATGCGGCATTCCAAACGAACTC, mCLK4 as-Not I, TATAGCGGCCGCCCTGACTCCCACTCATTTCCTTTTTAA. The cDNAs encoding the fusion construct were then recloned in pcDNA3 (Invitrogen) by PCR using the GST upstream primers: GST-EcoRI, CGGAATTCCGCCACCATGGCCCCTATACTAGGTTAT (for mCLK1) and GST-Hind III, GCCAAGCTTGCCACCATGGCCCCTATACTAGGTTAT (for mCLK2, mCLK3 and mCLK4).

Integrity of the clones was checked by sequencing and by a coupled transcription-translation assay using T7 RNA polymerase and rabbit reticulocyte lysate according to the manufacturer's protocol (Promega).

mCLK1-4 mutants containing a lysine (K) to arginine (R) substitution at position 190 (mCLK1), 192 (mCLK2), 186 (mCLK3) and 189 (mCLK4) were generated using a site-directed mutagenesis protocol. (Kunkel, PNAS 82:488-1985.) Oligonucleotide primers were as follows: (mCLK1-K190R) GTAGCAGTAAGAATAGTTAAA; (mCLK2-K192R) GTTGCCCTGAGGATCATTAAGAAT; (mCLK3-K186R) GTTGCCCTGAGGATCATCCGGAAT; (mCLK4-K189R) TACAATTCTCACTGCTACATGTAAGCCATC.

Human 293 cells were maintained in Dulbecco's modified Eagle's medium supplemented with 10% fetal calf serum. 3×10⁵ cells were seeded per 6 cm dish and transfected 24 hr later with 0.25-1 μg of DNA (cotrasfection of 0.5 μg of each plasmid described above) using the calcium precipitation method of Cehn and Okayama (Mol. Cell. Biol. 7:2745, 1987). These cells were used in the activity assays described below.

### Example 11

### Production of CLK-Specific Antibodies

Specific polyclonal antibodies were raised against each CLK protein using the C-terminal 17 amino acids of each CLK fused to keyhole limpet hemocyanin using standard protocols.

### Example 12

### Assay for Activity of CLKs

Glutathione S-transferase (GST) mCLK1-4 fusion constructs were generated to investigate the catalytic activity of these protein kinases. These protein kinases were cloned from pcDNA and expressed in vitro. The expression levels were almost identical and full-length fusion proteins of the expected molecular weights were obtained.

The transiently transfected 293 cells described in Example 10 above were seeded and grown as described. After 16 hr the medium was changed and the cells were incubated for another 6-48 hr (with or without 50 μM sodium orthovanadate) before lysis. Cells were lysed on ice for 30 min. in 200 μl HNTG buffer (50 mM HEPES, pH 7.5, 150 mM NaCl, 1% Triton X-100, 10% glycerol, 1 mM EDTA, 10 mM sodium fluoride, 5 mM β-glycerolphosphate, 1 mM phenylmethylsulfonyl fluoride, 1 μg/ml aprotinin). The cell lysates were centrifuged for 10 minutes at 4° C. and an equal volume of 2×SDS sample buffer added to the supernatant. 400 μl 1×SDS sample buffer was added, the samples were boiled for 5 min and 20 μl run on 10% SDS-PAGE gels. Following electrophoresis, the proteins were transferred to nitrocellulose membranes and immunoblotted with antibodies specific for the CLK proteins (see Example 11, supra) as well as anti-phosphotyrosine antibodies (4G10, Santa Cruz Biotech). CLKs 1-4 partitioned into a Triton X-100 soluble and insoluble fraction. The catalytically active kinases were tyrosine phosphorylated (via autophosphorylation) (as determined by the binding of 4G10) whereas the catalytically inactive mutants were not. These results suggest that each CLK is catalytically active.

The ability of CLK proteins to phosphorylate what may be a biologically relevant substrate, SR proteins, was also evaluated. 35S-methionine labeled GST-mCLK1-4 fusion proteins were produced in a 50 μl in vitro transcription/translation reaction using manufacturer's instructions (Promega). 2 μl of each reaction was checked and quantitated for the amounts of produced protein by SDS-PAGE and autoradiography. Equal amounts (usually 20-μl of lysate) were added to 500 μl PBS (1 mM PMSF, 10 μg/ml aprotinin), 30 μl of GSH-sepharose beads (Pharmacia) and incubated on a rotating wheel for 2 hours at 4° C. The beads were then washed three times in 500 μl PBS and once in 500 μl kinase assay buffer (20 mM Hepes, 10 mM MgCl2, 1 mM DTT, 200 μM sodium orthovanadate, 1 mM EGTA, pH 7.5). The assay was carried out for 30 minutes at room temperature in 30 μl kinase assay buffer with 20 μM ATP, 4 μCi gamma-³²P-ATP (Amersham, 10 mCi/ml) and approximately 2.5 μg of dephosphorylated SR proteins (prepared as described below). The reaction was stopped; by adding 30 μl of 2×SDS sample buffer. The samples were boiled for 5 min and 15 μl were loaded on a 15% SDS-PAGE gel. Following electrophoresis, the gels were stained, dried and exposed to Hyperfilm-MP (Amersham) for 24 hrs. The ³⁵S-methionine signal was suppressed with a 3M Whatman paper placed between the film and the gel.

All mCLK kinases were able to phosphorylate SRp2O, SRp2Oa and to a lesser extent SRp4O and SRp55. The lower signal of SRp4O and SRp55 relative to SRp2O and SRp3O most likely reflected the lower quantity of these proteins. SRp75 was not visualized in these experiments since the autophosphorylated mCLK proteins migrated at the same position. mCLK1 and mCLK4 phosphorylated SRp3Oa (upper band) more strongly than SRp3Ob, whereas mCLK2 and mCLK3 phosphorylated both with almost equal efficiency. A marked difference in catalytic activity was visualized between mCLK1 and mCLK4 versus mCLK2 and mCLK3, despite equal amounts of protein in each assay.

SR proteins were purified from 5×109 Friend murine erythroleukemia cells (F-MEL) according to the protocol described (Zahler et al., Genes Dev 6:837, 1992) and resuspended in buffer (D. Dignam et al., Nucleic Acids Res. 11:1475, 1 1983). 30 μl of SR proteins (C0.5 μg/μl) were incubated on ice for 10 minutes in 0.7 mM MnCl₂ and 5 mU Protein Phosphatase 1 gamma-catalytic subunit (Boehringer), followed by 60 minutes at 30° C. (Mermoud et al., EMBO J. 13:5679, 1994.) 5 μl of dephosphorylated SR proteins were used per assay.

### Example 13

### Identification and Cloning of SIRPs

### Materials and Methods—

MM5/C1, Rat1-IR, A431 or human fibroblast cells were grown until confluency, starved for 18 hours in serum-free medium, and either left untreated or were treated with POV—(1 mM sodium orthovanadate, 3 mM H2O2), insulin—(100 μM), EGF—(1 nM), or PDGF—(100 μM) for different time intervals. SIRP4, SHP-2 (Vogel, et al., Science 259:1611, 1994) or SHP-2C463A mutant (Stein-Gerlach, et al. J. Biol. Chem. 270:24635, 1995) cDNAS were transiently cotransfected in BHK-IR, BHK-EGFR or BHK-PDGFR cells using the calcium precipitation method (Chen, et al. Mol. Cell. Biol. 7:2745, 1987). After stimulation, cells were lysed in buffer containing 50 mM HEPES, pH 7.5, 150 mM NaCl, 1% Triton X-100, 10% glycerol, 1 mM POV, 1 mM EDTA, 1 mM PMSF, 1 mg/ml leupeptin, 1 mg/ml aprotinin.

SHP-2 immunoprecipitations were performed with polyclonal anti-SHP-2 antibodies (Vogel, et al., Science 259:1611, 1994). Western blots were labeled with monoclonal anti-phosphotyrosine antibodies 5E2 (Fendly, et al., Cancer Res. 50:1550, 1990), and after stripping, reprobed with monoclonal anti-SHP-2 antibodies (Transduction Laboratories). For immunolabeling goat anti-mouse or -rabbit horseradish peroxidase conjugates (Bio-Rad) and the ECL detection system (Amersham) were used.

To perform in vitro deglycosylation SHP-2 immunocomplexes or the 110 kDa protein preparation were first denatured in the presence of 1% SDS at 100° C. for 5 min. Deglycosylation was done in potassium phosphate buffer (40 mM, pH 7.0), containing 20 mM EDTA, 1% β-mercaptoethanol, 1% Triton X-100 and 0.5 Unit of Endoglycosidase F/N-Glycosidase F (Boehringer Mannheim) at 37° C. for 16 hours.

To obtain purified SHP2 binding protein approximately 10¹⁰ Rat1-IR cells were used to purify the 110 kDa protein. Starved Rat1-IR cells were insulin-stimulated (100 nM) for 10 min, washed briefly with ice-cold hypotonic buffer containing 20 mM HEPES, pH 7.5, 1 mM POV, 1 mM EDTA, 1 mM PMSF, 1 mg/ml leupeptin, 1 mg/ml aprotinin, scraped into the same buffer and homogenized. Cell extracts were pelleted at 1000 rpm for 15 min, and supernatants were spun at 48.000 g for 1 hour. Membranes were solubilized in lysis buffer as described above. hIR was depleted from membrane extracts using an affinity column with monoclonal anti-hIR antibody 83-14 (Redemann et al., Mol. Cell. Biol. 12:491, 1992), covalently coupled to Protein A-Sepharose beads (Pharmacia). Depleted extracts were applied onto a WGA-agarose 6 MB column (Sigma), and glycoproteins were eluted with 0.3 M N-acetyl-glucosamine in HNTG (20 mM HEPES (pH 7.5), 150 mM NaCl, 0.1% Triton X-100, 10% glycerol, 1 mM POV). After concentration protein extracts were applied onto an anti-phosphotyrosine antibody column (Sigma). Bound proteins were eluted with 20 mM phosphotyrosine in HNTG. The eluate was subjected to SDS-PAGE, proteins were transferred to a PVDF membrane (Millipore) and stained with Coomassie blue.

### Results—

Western blot of mammalian cells with anti-phosphotyrosine antibodies and anti-SHP-2 antibodies was used to identify tyrosine phosphorylated SHP-2 associated proteins.

Western blots containing anti-SHP-2 immunoprecipitates from starved or POV-treated mouse MM5/C1 mammary carcinoma, rat fibroblast Rat1-IR or human epidermal carcinoma A431 cells were incubated with anti-phosphotyrosine antibodies or anti-SHP-2 antibodies. Samples were deglycosylated with or treated without Endoglycosidase F/N-Glycosidase F (Endo. F/F). As a control, insulin-stimulated Rat1-IR cell lysates were immunoprecipitated with preimmune rabbit serum (aNS).

Samples from each purification step (i.e., solubilized crude membrane extract, hIR-depleted extracts, concentrated eluate from WGA-agarose beads, and eluate from anti-phosphotyrosine antibody column) were analyzed by 10% SDS-PAGE and visualized by silver staining and in Western blots using monoclonal anti-phosphotyrosine antibodies.

A major tyrosine phosphorylated protein was revealed in analysis of anti-SHP-2 immunoprecipitates from both pervanadate (POV) and growth factor stimulated cells. This phosphoprotein migrated at 120 kDa, 110 kDa and 90 kDa positions in mouse mammary tumor (MM5/C1) cells, Rat1 cells overexpressing the human insulin receptor (Rat1-IR), and human epidermoid carcinoma (A431) cells, respectively.

Upon in vitro deglycosylation, this glycoprotein was reduced to 65 kDa apparent molecular weight (MW) in all cases. This indicated that the same SHP-2 binding protein of 65 kDa was differentially glycosylated in a species specific manner.

In some cell lines such as A431, other tyrosine phosphorylated proteins in the 90-120 kDa range remained unaffected by the deglycosylation treatment. These proteins may represent Gab1 and/or the human homologue of the Drosophila DOS protein.

Insulin treated Rat1-IR were used to purify the 110 kDa SHP-2 binding glycoprotein using standard chromatography procedures. Approximately 4 mg of the glycoprotein that copurified with SHP-2 were obtained and subject to microsequence analysis. This yielded five peptide sequences: PIYSFIGGEHFPR, IVEPDTEIK, YGFSPR, IKEVAHVNLEVR, VAAGDSAT. Computer aided search in the EST database led to the identification of a 305 bp rat sequence (accession Nr.: H31804) and subsequent human cDNA fragment of 2 kb (EMBL databank, accession Nr.: U6701) containing matching and homologous sequences, respectively.

Specific primers flanking the very 5′ portion of this sequence were used to amplify a 360 bp human DNA fragment which was used to screen a human placenta cDNA library.

Several positive clones were isolated. One clone of 2.4 kb encoded a polypeptide of 503 amino acids designated SIRP4 (for SIgnal Regulating Protein 4) with a calculated mass of 57,000. The deduced sequence identifies SIRP4 as a transmembrane protein with three Ig-like domains and a cytoplasmic portion containing four potential tyrosine phosphorylation sites and one proline-rich region.

A second cDNA clone, SIRP1, is also identified. This protein is highly homologous to SIRP4 within the Ig-like domains (Ig-1: 83%; Ig-2: 88%; Ig-3: 83%), but displays striking sequence divergence at the amino terminus and upstream of the transmembrane domain which gives rise to a shorter protein that still contains a transmembrane-like region but lacks the cytoplasmic C-terminal portion.

SIRP4 and SIRP1 are members of a novel protein family. This protein family has a variety of distinct sequence isoforms as evidenced by comparison of fifteen cDNA and genomic sequences within the first Ig-like domain. Two major classes exist in SIRP family distinguished by the presence or absence of a cytoplasmic SHP-2 binding domain.

### Example 14

### Production of SIRP-Specific Antibodies

Polyclonal anti-SIRP antibodies were raised by immunizing rabbits with a GST-fusion protein containing a fragment of the SRIP4 amino acid sequence (aa 33-139) or containing the C-terminal part of SIRP4 (amino acids 336-503).

### Example 15

### Recombinant Expression of SIRPs

To obtain 293 cells stably expressing SIRP4 (293/SIRP4), cells were transfected with SIRP4 cDNA in pLXSN (Miller, et al. Biotechniques 7:980, 1989) using the calcium precipitation method, followed by selection with G418 (1 mg/ml). SIRP4 was immunoprecipitated from quiescent or POV-stimulated (1 mM) 293/SIRP4 cells with polyclonal anti-SIRP4 antibodies (see Example 14, infra). Subsequently, crude lysates of [35S]-methionine labeled 293 cells expressing different SH2 domain containing proteins were added to the affinity matrix and incubated for 2 h at 4 oC. The immunocomplexes were washed, separated by SDS-PAGE and analyzed by autoradiography.

To produce retroviruses expressing pLXSN, wild type SIRP4 and mutated SIRP4 constructs, BOSC 23 cells were transiently transfected by expression plasmids as described (Pear, et al. Proc. Natl. Acad. Sci. 90:8392, 1993). To obtain NIH3T3 cells stably expressing wild type SIRP4, SIRP4-4Y or SIRP4-DCT mutants subconfluent NIH3T3 cells (10⁵ cells per 6 cm dish) were incubated with supernatants of transfected BOSC 23 cells for 4 h in the presence of Polybrene (4 mg/ml), followed by selection with G418 (1 mg/ml).

To perform focus formation assays cell lines 3T3/pLXSN, 3T3/SIRP4, 3T3/SIRP4-4Y or 3T3/SIRP4-DCT were superinfected for 4 hours with equal volumes of v-fms-virus supernatant (10⁵ cells/6 cm dish). Cells were cultivated for 14 days in 4% FCS with medium change every second day. Cell foci were stained with Crystal violet (0.1% crystal violet, 30% methanol).

The identity of SIRP4 as SHP-2 binding protein and substrate was confirmed by expression of the SIRP4 cDNA either alone or in combination with SHP-2 or an enzymatically inactive mutant SHP-2C463A in BHK cells. BHK cells stably express human EGF-, insulin- or PDGF receptors. Anti-SIRP4 immunoprecipitation revealed a tyrosine phosphorylated protein of 85-90 kDa upon ligand stimulation which associated with SHP-2.

The results suggested SIRP4 to be a direct substrate of SHP-2 since expression of the SHP-2 mutant SHP-2C463A led to a significant increase in its phosphotyrosine content (even in starved cells) while coexpression of wt SHP-2 resulted in dephosphorylation. The MW of overexpressed SIRP4 matches that of the endogenous protein detected in SHP-2 immunoprecipitates from A431 cells.

### Example 16

### Endogenous Expression of SIRPs

Endogenous SIRP4-like proteins were immunoprecipitated from untreated or EGF-stimulated A431 cells, from quiescent or PDGF-treated human fibroblasts, or from starved or insulin-stimulated HBL-100 cells. As a control, ligand-stimulated cell lysates were immunoprecipitated with preimmune rabbit serum (aNS). Immunoblots were probed with monoclonal anti-phosphotyrosine and monoclonal anti-SHP-2 antibodies.

Polyclonal anti-SIRP antibodies immunoprecipitate a protein of 85-90 kDa apparent MW from A431, HBL-100 tumor cells and human fibroblasts. This protein was tyrosine phosphorylated upon EGF, insulin or PDGF stimulation, respectively, and coprecipitated with SHP-2 in a ligand dependent manner.

These data indicate the existence of SIRP4 in several human cell lines where SIRP4 serves as a substrate for insulin-, EGF- and PDGF receptors, binds SHP-2 in its tyrosine phosphorylated form and serves as a substrate for the phosphatase activity of SHP-2. The interaction of SHP-2 with SIRP4 likely involves one or both SH2 domains of SHP-2 as suggested by the requirement of phosphotyrosine residues and the abrogation of detectable association by mutation of critical residues in SHP-2 SH2 domains.

In vitro binding assays were performed to determine whether SIRP4 is able to interact with other SH2 domain-containing proteins. SIRP4-associated [³⁵S]-Methionine labeled proteins were resolved on SDS-PAGE and detected by autoradiography. The result shows that SIRP4 associates with both SHP-1 and Grb2 but not p85, Shc, Grb7, PLC-g, c-src, Nck, Vav, GAP, or ISGF-3.

### Example 17

### Effects of SIRP4 on Cell Growth and Transformation

To investigate the biological function of SIRP4, three stable transfectants of NIH3T3 cells were constructed to express wild type SIRP4 or SIRP4 mutants carrying either point mutations of the putative SHP-2 tyrosine binding sites (SIRP4-4Y) or a deletion of most of the cytoplasmic region (SIRP4-DCT) (see Examples above).

Ligand-stimulated [3H]-thymidine incorporation of NIH3T3 cells expressing empty vector (3T3/pLXSN), wild type SIRP4 (3T3/SIRP4), SIRP4-4Y (3T3/SIRP4-4Y) or SIRP4-DCT (3T3/SIRP4-DCT, amino acids 402-503 are deleted) mutants. Cells were grown to confluence in 24-well dishes (Nunc), starved for 24 h in DMEM/0.5% FCS, stimulated with different concentrations of insulin or EGF for 18 h, then incubated with 0.5 mCi [³H]-thymidine per well for 4 h. Incorporation into DNA was determined as described (Redemann, et al. Mol. Cell. Biol. 12:491, 1992).

Upon stimulation of cells with insulin, EGF and PDGF, control cells showed growth factor-induced DNA synthesis as measured by [³H]-thymidine incorporation. Overexpression of SIRP4 led to a decrease of [³H]-thymidine incorporation. In contrast, both SIRP4 mutants had nearly no effect on DNA synthesis. The observed inhibitory effect on DNA synthesis must be connected to SIRP4 tyrosine phosphorylation and/or its association with SHP-2 since wt SIRP4 became tyrosine phosphorylated and bound to SHP-2 upon ligand stimulation, and SIRP4 mutants did not.

SIRP4 effected growth inhibition upon insulin or EGF stimulation is correlated with reduced MAP kinase activation in 3T3/SIRP4 cells. 3T3/pLXSN, 3T3/SIRP4 or 3T3/SIRP4-4Y cells were starved for 18 hours in DMEM/0.5% FCS and stimulated with insulin or EGF for the time indicated. MAP kinase was detected in Western blots by using polyclonal erk1 and erk2 antibodies (Santa Cruz). In contrast, expression of SIRP4 mutants defective in SHP-2 binding had no effect on MAP kinase activation. Similar observations were made upon stimulation of the cells with PDGF.

These data strongly indicate that SIRP4 represents a novel regulatory element in the pathway that leads to MAP kinase activation.

We next determined the consequence of SIRP4 overexpression on oncogene mediated transformation of NIH3T3 cells. To examine the ability of SIRP4 to influence the formation of cell foci, subconfluent 3T3/pLXSN, 3T3/SIRP4, 3T3/SIRP4-4Y or 3T3/SIRP4-DCT cells were infected with v-fms virus supernatants.

As measured by focus formation, transformation by a v-fms retrovirus was significantly suppressed in cells overexpressing wt SIRP4 but not in cells expressing mutant SIRP4.

Previous reports have described certain SHP-2 binding proteins of 110-130 kDa apparent MW in mouse, rat or hamster cells. Tyrosine hyperphosphorylation of these proteins was observed when an enzymatically inactive SHP-2 mutant was overexpressed. In addition, disruption of SHP-2 function induced a variety of negative effects on growth factor-induced cellular signals. Our experiments strongly indicate that these proteins belong to the SIRP family and that the biological effects previously observed are due to the function of these SIRP proteins.

Without being bound by any theory, applicant proposes that tyrosine docking sites on SIRP proteins for either SHP-2 and/or other SH2 proteins such as SHP-1 or Grb2 play a significant role since the inhibitory effect of SIRP4 on NIH3T3 cell proliferation and transformation depends on phosphorylation of tyrosines. One or both of the SHP phosphatases may tightly regulate the SIRP4 phosphorylation state. SIRP4 may also act in its phosphorylated state as a “trapping” protein that sequesters SHP-2 from activated RTKs. The sequestion makes SHP-2 unavailable for other positive regulatory functions such as an adapter which recruits the Grb2-SOS complex to activated receptors. Such a function is supported by the observation that SHP-2 has higher affinity to the tyrosine phosphorylated form of SIRP4 than to autophosphorylated insulin and EGF receptors (Yamauchi, et al., J. Biol. Chem. 270:17716-17722, Yamauchi, et al. J. Biol. Chem. 270:14871-14874 (1995)).

A third possibility is based on the membrane-spanning structural features of the SIRP4 variant. The high degree of sequence diversity within the Ig-domains is reminiscent of immunoglobulin variable regions and suggests a role of extracellular determinants in the SIRP related signal transduction. Structurally defined interaction of SIRP with specific receptors, soluble ligands, extracellular matrix components or other factors may result in specific regulatory consequences for intracellular signaling events.

Although certain embodiments and examples have been used to describe the present invention, it will be apparent to those skilled in the art that changes to the embodiments and examples shown may be made without departing from the scope or spirit of the invention.

Those references not previously incorporated herein by reference, including both patent and non-patent references, are expressly incorporated herein by reference for all purposes.

Other embodiments are encompassed by the following claims.

1. An isolated, enriched, or purified nucleic acid molecule encoding a PTP20, PCP-2, BDP1, mCLK2, mCLK3, mCLK4, or SIRP polypeptide.

2-8. (canceled)

9. An isolated, enriched or purified PTP20, PCP-2, BDP1, mCLK2, mCLK3, mCLK4, or SIRP polypeptide.

10-12. (canceled)

13. An antibody or an antibody fragment having specific binding affinity to a PTP20, PCP-2, BDP1, mCLK2, mCLK3, mCLK4, or SIRP polypeptide.

14. The antibody of claim 13 wherein said polypeptide comprises at least 4 contiguous amino acids of the amino acid sequence shown in FIG. 1, 2, 3, 4, or 5.

15-27. (canceled)
